(19) United States
(12) Patent Application Publication
(10) Pub. No.: US 2008/0108600 A1
(43) Pub. Date: May 8, 2008

(54) Polycyclic Pyridines as Potassium Ion Channel Modulators

(75) Inventors: Xiaodong Wang, Chapel Hill, NC (US); Kerry L. Spear, Concord, MA (US); Alan B. Fulp, Willow Springs, NC (US); Darrick Seconi, Cary, NC (US); Takeshi Suzuki, Ibaraki (JP); Takahiro Ishii, Ibaraki (JP); Ayako Moritomo, Ibaraki (JP); Hideki Kubota, Ibaraki (JP); Jun-ichi Kazami, Ibaraki (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Icagen, Inc., Durham, NC (US); Astellas Parma Inc., Ibaraki (JP)

(21) Appl. No.: 10/592,985

(22) PCT Filed: Apr. 13, 2005
(86) PCT No.: PCT/US05/12837

§ 371 Date: Sep. 12, 2007

Related U.S. Application Data
(60) Provisional application No. 60/562,035, filed on Apr. 13, 2004.

(51) Int. Cl. A61K 31/444; C12N 5/00; A61P 25/00; A61P 1/00; A61P 9/00; A61K 31/496; A61K 31/551; A61K 31/5377; A61K 31/497; C07D 417/14; C07D 401/14; A61K 31/4725

(52) U.S. Cl. 514/218; 435/375; 546/256; 546/139; 514/307; 514/333; 514/255.05; 544/405; 544/364; 514/253.01; 514/253.1; 514/236.8; 544/124; 540/575

(57) ABSTRACT

The present invention provides a genus of polycyclic pyridines that are useful as modulators of potassium ion channels. The modulators of the invention are of use in both therapeutic and diagnostic methods.

### CROSS-REFERENCES TO RELATED APPLICATIONS

This application claims the benefit of U.S. Provisional Patent Application No. 60/562,035, filed Apr. 13, 2004, which is incorporated herein by reference in its entirety for all purposes.

### BACKGROUND OF THE INVENTION

Ion channels are cellular proteins that regulate the flow of ions, including calcium, potassium, sodium and chloride into and out of cells. These channels are present in all human cells and affect such physiological processes as nerve transmission, muscle contraction, cellular secretion, regulation of heartbeat, dilation of arteries, release of insulin, and regulation of renal electrolyte transport. Among the ion channels, potassium ion channels are the most ubiquitous and diverse, being found in a variety of animal cells such as nervous, muscular, glandular, immune, reproductive, and epithelial tissue. These channels allow the flow of potassium in and/or out of the cell under certain conditions. For example, the outward flow of potassium ions upon opening of these channels makes the interior of the cell more negative, counteracting depolarizing voltages applied to the cell. These channels are regulated, e.g., by calcium sensitivity, voltage-gating, second messengers, extracellular ligands, and ATP-sensitivity.

Potassium ion channels are typically formed by four alpha subunits, and can be homomeric (made of identical alpha subunits) or heteromeric (made of two or more distinct types of alpha subunits). In addition, certain potassium ion channels (those made from Kv, KQT and Slo or BK subunits) have often been found to contain additional, structurally distinct auxiliary, or beta subunits. These subunits do not form potassium ion channels themselves, but instead they act as auxiliary subunits to modify the functional properties of channels formed by alpha subunits. For example, the Kv beta subunits are cytoplasmic and are known to increase the surface expression of Kv channels and/or modify inactivation kinetics of the channel (Heinemann et al., J. Physiol. 493: 625-633 (1996); Shi et al., Neuron 16(4): 843-852 (1996)). In another example, the KQT family beta subunit, minK, primarily changes activation kinetics (Sanguinetti et al., Nature 384: 80-83 (1996)).

The alpha subunits of potassium ion channels fall into at least 8 families, based on predicted structural and functional similarities (Wei et al., Neuropharmacology 35(7): 805-829 (1997)). Three of these families (Kv, eag-related, and KQT) share a common motif of six transmembrane domains and are primarily gated by voltage. Two other families, CNG and SK/IK, also contain this motif but are gated by cyclic nucleotides and calcium, respectively. Small (SK) and intermediate (IK) conductance calcium-activated potassium ion channels possess unit conductances of 2-20 and 20-85 pS, respectively, and are more sensitive to calcium than are BK channels discussed below. For a review of calcium-activated potassium channels see Latorre et al., Ann. Rev. Phys. 51: 385-399 (1989).

Three other families of potassium channel alpha subunits have distinct patterns of transmembrane domains. Slo or BK family potassium channels have seven transmembrane domains (Meera et al., Proc. Natl. Acad. Sci. U.S.A. 94(25): 14066-14071 (1997)) and are gated by both voltage and calcium or pH (Schreiber et al., J. Biol. Chem. 273: 3509-3516 (1998)). Slo or BK potassium ion channels are large conductance potassium ion channels found in a wide variety of tissues, both in the central nervous system and periphery. These channels are gated by the concerted actions of internal calcium ions and membrane potential, and have a unit conductance between 100 and 220 pS. They play a key role in the regulation of processes such as neuronal integration, muscular contraction and hormone secretion. They may also be involved in processes such as lymphocyte differentiation and cell proliferation, spermatocyte differentiation and sperm motility. Members of the BK (Atkinson et al., Science 253: 551-555 (1991); Adelman et al., Neuron 9: 209-216 (1992); Butler, Science 261: 221-224 (1993)) subfamily have been cloned and expressed in heterologous cell types where they recapitulate the fundamental properties of their native counterparts. Finally, the inward rectifier potassium channels (Kir), belong to a structural family containing two transmembrane domains, and an eighth functionally diverse family (TP, or “two-pore”) contains two tandem repeats of this inward rectifier motif.

Each type of potassium ion channel shows a distinct pharmacological profile. These classes are widely expressed, and their activity hyperpolarizes the membrane potential. Potassium ion channels have been associated with a number of physiological processes, including regulation of heartbeat, dilation of arteries, release of insulin, excitability of nerve cells, and regulation of renal electrolyte transport. Moreover, studies have indicated that potassium ion channels are a therapeutic target in the treatment of a number of diseases including central or peripheral nervous system disorders (e.g., migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases), as well as targets for neuroprotective agents (e.g., to prevent stroke and the like); as well as disease states such as gastroesophogeal reflux disorder and gastrointestinal hypomotility disorders, irritable bowel syndrome, secretory diarrhea, asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomia, diabetes type II, hyperinsulinemia, premature labor, baldness, cancer, and immune suppression.

Specifically, SK channels have been shown to have distinct pharmacological profiles. For example, using patch clamp techniques, the effects of eight clinically relevant psychoactive compounds on SK2 subtype channels were investigated (Dreixler et al., Eur. J. Pharmacol. 401: 1-7 (2000)). The evaluated compounds are structurally related to tricyclic antidepressants and include amitriptyline, carbamazepine, chlorpromazine, cyproheptadine, imipramine, tacrine and trifluperazine. Each of the compounds tested was found to block SK2 channel currents with micromolar affinity. A number of neuromuscular inhibiting agents exist that affect SK channels, e.g. apamin, atracurium, pancuronium and tubocurarine (Shah et al., Br J Pharmacol 129: 627-30 (2000)).

Moreover, patch clamp techniques have also been used to study the effect of the centrally acting muscle relaxant chlorzoxazone and three structurally related compounds, 1-ethyl-2-benzimidazolinone (1-EBIO), zoxazolamine, and 1,3-dihydro-1-[2-hydroxy-5-(trifluoromethyl)phenyl]-5-(trifluoromethyl)-2H-benzimidazol-2-one (NS 1619) on recombinant rat brain SK2 channels (rSK2 channels) expressed in HEK293 mammalian cells (Cao et al., J Pharmacol. Exp. Ther. 296: 683-689 (2001)). When applied externally, chlorzoxazone, 1-EBIO, and zoxazolamine activated rSK2 channel currents in cells dialyzed with a nominally calcium-free intracellular solution.

The effects of metal cations on the activation of recombinant human SK4 (also known as hIK1 or hKCa4) channels has also been studied (Cao and Houamed, FEBS Lett. 446: 137-41 (1999)). The ion channels were expressed in HEK 293 cells and tested using patch clamp recording. Of the nine metals tested, cobalt, iron, magnesium, and zinc did not activate the SK4 channels when applied to the inside of SK4 channel-expressing membrane patches. Barium, cadmium, calcium, lead, and strontium activated SK4 channels in a concentration-dependent manner. Calcium was the most potent metal, followed by lead, cadmium, strontium, and barium.

The SK channels are heteromeric complexes that comprise pore-forming α-subunits and the calcium binding protein calmodulin (CaM). CaM binds to the SK channel through the CaM-binding domain (CaMBD), which is located in an intracellular region of an α-subunit close to the pore. Based on a recently published crystal structure, calcium binding to the N-lobe of the CaM proteins on each of the four subunits initiates a structural change that allows a hydrophobic portion of the CaM protein to interact with a CaMBD on an adjacent subunit. As each N-lobe on an adjacent subunit grabs the other CaMBD C-terminal region, a rotary force is thought to be created between them which would drive open the channel.

New classes of compounds that act to modulate the opening of potassium ion channels would represent a significant advance in the art and provide the opportunity to develop treatment modalities for numerous diseases associated with these channels. The present invention provides a new class of potassium ion channel modulators and methods of using the modulators.

### BRIEF SUMMARY OF THE INVENTION

The present invention provides polycyclic pyridines, prodrugs, complexes and pharmaceutically acceptable salts thereof, which are useful in the treatment of diseases through the modulation of potassium ion flow through potassium ion channels.

In one aspect, the potassium ion channel modulator has the structure according to Formula (I):

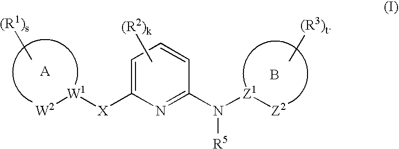

In Formula (I), A and B are independently substituted or unsubstituted 5- or 6-membered rings. In some embodiments, A and B are independently 5- or 6-membered heterocycloalkyl or 5- or 6-membered heteroaryl.

The symbol W¹ is

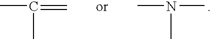

### W² is —CH═, —NH—, —N═, or —O—. The

symbol Z¹ is

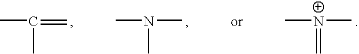

### Z² is —CH═, —NH—, —N═, or —O—. In some

embodiments, W¹ and Z¹ are independently

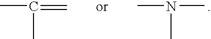

### W² and Z² may independently be —NH— or —N═. The symbol X is a bond, —CH₂—, or —NR⁴—.

The symbols s and t are independently integers from 1 to 4. The symbol k is an integer from 1 to 3.

R¹, R², and R³ are independently H, —NO₂, —CF₃, -L¹-OR⁶, -L²-NR⁷R′, -L¹-CONR⁷R⁸, -L⁴-COOR⁶, -L⁵-COR⁶, -L⁶-SO₂R⁶, -L⁷-SO₂NR⁷R⁸, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

R⁴ and R⁵ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, -L³-CONR⁷R⁸, -L⁴-COOR⁶, -L⁵-COR⁶, -L⁶-SO₂R⁶, or -L⁷-SO₂NR⁷R⁸.

L¹, L², L³, L⁴, L⁵, L⁶, and L⁷ are independently a bond, or substituted or unsubstituted (C₁-C₆) alkylene.

R⁶ is H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

R⁷ and R⁸ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, —COR⁸¹, or —SO₂R⁸¹. R⁷ and R⁸ are optionally joined with the nitrogen to which they are attached to form a substituted or unsubstituted 5- to 7-membered heterocycloalkyl, or substituted or unsubstituted heteroaryl.

R⁸¹ is substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

Where a plurality of R groups or L groups are present, each group is optionally different.

In a second aspect, the present invention provides a method for decreasing ion flow through potassium ion channels in a cell, comprising contacting the cell with a potassium ion channel modulating amount of a potassium channel modulator of the present invention.

In a third aspect, the present invention provides a method for treating a disease through the modulation of potassium ion flow through these channels. The modulators are useful in the treatment of central or peripheral nervous system disorders (e.g., migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases), and as neuroprotective agents (e.g., to prevent stroke and the like). The modulators of the invention are also useful in treating disease states such as gastroesophogeal reflux disorder and gastrointestinal hypomotility disorders, irritable bowel syndrome, secretory diarrhea, asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomi, diabetes type II, hyperinsulinemia, premature labor, baldness, cancer, and immune suppression. This method involves administering, to a patient, an effective amount of a potassium channel modulator of the present invention.

In a fourth aspect, the present invention provides a pharmaceutical composition comprising a pharmaceutically acceptable carrier and a potassium channel modulator of the present invention.

These and other aspects and embodiments of the invention will be apparent from the detailed description that follows.

### DETAILED DESCRIPTION OF THE INVENTION

### I. Abbreviations and Definitions

The abbreviations used herein have their conventional meaning within the chemical and biological arts.

Where moieties are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ or 1- to 10-membered means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—C(═O)—CH₃, —CH₂—CH₂—CH₂—C(═O)—O—C(CH₃)—CH₃, —CH₂—CH₂—CH₂—C(═O)—N—CH(CH₃), —CH₂—CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated and unsaturated ring linkages. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholino, 3-morpholino, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furanyl, 3-furanyl, 2-thienyl, 3-thienyl, 2-pyridinyl, 3-pyridinyl, 4-pyridinyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1 to 3 halogens, substituted or unsubstituted alkyl, alkoxy or thioalkoxy groups, or arylalkyl groups. When a modulator of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholino. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, alkyl, heteroalkyl, aryl and heteroaryl. When a modulator of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)_{S}—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R″″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

A “substituent group,” as used herein, means a group selected from the following moieties:
(A) —OH, —NH₂, —SH, —CN, —CF₃, oxy, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxy, —OH, —NIH2, —SH, —CN, —CF₃, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2- to 20-membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active modulators which are prepared with relatively nontoxic acids or bases, depending on the particular substituents found on the modulators described herein. When modulators of the present invention contain relatively acidic functionalities, base addition salts can be obtained by contacting the neutral form of such modulators with a sufficient amount of the desired base, either neat or in a suitable inert solvent. Examples of pharmaceutically acceptable base addition salts include sodium, potassium, calcium, ammonium, organic amino, or magnesium salt, or a similar salt. When modulators of the present invention contain relatively basic functionalities, acid addition salts can be obtained by contacting the neutral form of such modulators with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific modulators of the present invention contain both basic and acidic functionalities that allow the modulators to be converted into either base or acid addition salts.

The neutral forms of the modulators are preferably regenerated by contacting the salt with a base or acid and isolating the parent modulator in the conventional manner. The parent form of the modulator differs from the various salt forms in certain physical properties, such as solubility in polar solvents.

In addition to salt forms, the present invention provides modulators, which are in a prodrug form. Prodrugs of the modulators described herein are those compounds or complexes that readily undergo chemical changes under physiological conditions to provide the modulators of the present invention. Additionally, prodrugs can be converted to the modulators of the present invention by chemical or biochemical methods in an ex vivo environment. For example, prodrugs can be slowly converted to the modulators of the present invention when placed in a transdermal patch reservoir with a suitable enzyme or chemical reagent.

The term “ring” as used herein means a substituted or unsubstituted cycloalkyl, substituted or unsubstituted heterocycloalkyl, substituted or unsubstituted aryl, or substituted or unsubstituted heteroaryl. A ring includes fused ring moities. The number of atoms in a ring are typically defined by the number of members in the ring. For example, a “5- to 7-membered ring” means there are 5-7 atoms in the encircling arrangement. The ring optionally includes a heteroatom. Thus, the term “5- to 7-membered ring” includes, for example pyridinyl, piperidinyl and thiazolyl rings.

The term “poly” as used herein means at least 2. For example, a polyvalent metal ion is a metal ion having a valency of at least 2.

“Moiety” refers to the radical of a molecule that is attached to another moiety.

The symbol [symbol] , whether utilized as a bond or displayed perpendicular to a bond indicates the point at which the displayed moiety is attached to the remainder of the molecule.

Certain modulators of the present invention can exist in unsolvated forms as well as solvated forms, including hydrated forms. In general, the solvated forms are equivalent to unsolvated forms and are encompassed within the scope of the present invention. Certain modulators of the present invention may exist in multiple crystalline or amorphous forms. In general, all physical forms are equivalent for the uses contemplated by the present invention and are intended to be within the scope of the present invention.

Certain modulators of the present invention possess asymmetric carbon atoms (optical centers) or double bonds; the racemates, diastereomers, geometric isomers and individual isomers are encompassed within the scope of the present invention.

The modulators of the present invention may also contain unnatural proportions of atomic isotopes at one or more of the atoms that constitute such modulators. For example, the modulators may be radiolabeled with radioactive isotopes, such as for example tritium (³H), iodine-125 (¹²⁵I) or carbon-14 (¹⁴C). All isotopic variations of the modulators of the present invention, whether radioactive or not, are encompassed within the scope of the present invention.

### II. Potassium Ion Channel Modulators

The invention provides potassium ion channel modulators that include a pyridinyl moiety and a first and a second ring, each of said rings being attached, either directly or through a linker, to the pyridinyl moiety. A potassium ion channel modulator of the present invention (“modulator of the present invention”) may be a compound (also referred to herein as a “compound of the present invention”) or metal ion complex (also referred to herein as a “complex of the present invention”), as described below.

In one embodiment, the potassium ion channel modulator has a structure according to Formula (I):

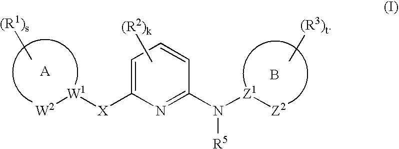

In Formula (I), A and B are independently substituted or unsubstituted 5- or 6-membered rings. In some embodiments, A and B are independently 5- or 6-membered heterocycloalkyl or 5- or 6-membered heteroaryl.

The symbol W¹ is

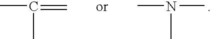

### W² is —CH═, —NH—, —N═, or —O—. The symbol Z¹ is

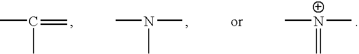

### Z² is —CH═, —NH—, —N═, or —O—. In some embodiments, W¹ and Z¹ are independently

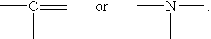

### W² and Z² may independently be —NH— or —N═. In other embodiments, Z¹ is

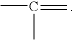

### Z² may be —N═.

The symbol X is a bond, —CH₂—, or —NR⁴—. In some embodiments, X is a bond or —NR⁴. X may also be a bond.

The symbols s and t are independently integers from 1 to 4. One of skill in the art will immediately recognize that where A is a 5-membered heterocycloalkyl or 5-membered heteroaryl, then s is an integer from 1 to 3; and where A is a 6-membered heterocycloalkyl or 6-membered heteroaryl, then s is an integer from 1 to 4. Likewise, where B is a 5-membered heterocycloalkyl or 5-membered heteroaryl, then t is an integer from 1 to 3 and where B is a 6-membered heterocycloalkyl or 6-membered heteroaryl, then t is an integer from 1 to 4.

The symbol k is an integer from 1 to 3.

R¹, R², and R³, are independently H, —NO₂, —CF₃, -L¹-OR⁶, -L²-NR⁷R⁸, -L³-CONR⁷R⁸, -L⁴-COOR⁶, -L⁵-COR⁶, -L⁶-SO₂R⁶, -L⁷-SO₂NR⁷R⁸, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

R⁴ and R⁵ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, -L³-CONR⁷R⁸, -L⁴-COOR⁶, -L⁵-COR⁶, -L⁶-SO₂R⁶, or -L⁷-SO₂NR⁷R⁸.

L¹, L², L³, L⁴, L⁵, L⁶, and L⁷ are independently a bond, or substituted or unsubstituted (C₁-C₆) alkylene.

R⁶ is H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

R⁷ and R⁸ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, —COR⁸¹, or —SO₂R⁸¹. R⁷ and R⁸ are optionally joined with the nitrogen to which they are attached to form a substituted or unsubstituted 5- to 7-membered heterocycloalkyl or substituted or unsubstituted heteroaryl.

R⁸¹ is substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl.

Where a plurality of R¹, R², R³, R⁶, R⁷, R⁸, R⁸¹, L, L², L³, L⁴, L, L⁶, and/or L⁷ groups are present, each R¹, R², R³, R⁶, R⁷, R⁸, R⁸¹, L¹, L², L³, L⁴, L⁵, L⁶, and/or L⁷ group is optionally different. For example, where s is greater than one, then each R¹ is optionally different; where k is greater than one, then each R² is optionally different; and where t is greater than one, then each R³ is optionally different.

R¹, R², and R³ may optionally form part of a fused ring system, either with themselves or with other groups. For example, two R¹ groups are optionally joined together with the atoms to which they are attached to form a substituted or unsubstituted 5- to 7-membered ring; two R² groups are optionally joined together with the atoms to which they are attached to form a substituted or unsubstituted 5- to 7-membered ring; two R³ groups are optionally joined together with the atoms to which they are attached to form a substituted or unsubstituted 5- to 7-membered ring; R¹ and R² are optionally joined together with the atoms to which they are attached to form a substituted or unsubstituted 5- to 7-membered ring; R² and R⁴ are optionally joined together with the atoms to which they are attached to form a substituted or unsubstituted 5- to 7-membered ring; R² and R⁵ are optionally joined together with the atoms to which they are attached to form a substituted or unsubstituted 5- to 7-membered ring; R² and R³ are optionally joined together with the atoms to which they are attached to form a substituted or unsubstituted 5- to 7-membered ring; R¹ and X (e.g. R⁴) are optionally joined together with the atoms to which they are attached to form a substituted or unsubstituted 5- to 7-membered ring; R² and X (e.g. R⁴) are optionally joined together with the atoms to which they are attached to form a substituted or unsubstituted 5- to 7-membered ring; R² and R⁵ are optionally joined together with the atoms to which they are attached to form a substituted or unsubstituted 5- to 7-membered ring; and R³ and R⁵ are optionally joined together with the atoms to which they are attached to form a substituted or unsubstituted 5- to 7-membered ring.

In some embodiments, two R¹ groups are optionally joined together with the atoms to which they are attached to form a substituted or unsubstituted 5- to 7-membered ring; two R² groups are optionally joined together with the atoms to which they are attached to form a substituted or unsubstituted 5- to 7-membered ring; and two R³ groups are optionally joined together with the atoms to which they are attached to form a substituted or unsubstituted 5- to 7-membered ring.

In some embodiments, R¹ is H, —OR⁶, —NR⁷R⁸, —NO₂, halogen, substituted or unsubstituted (C₁-C₅) alkyl, substituted or unsubstituted 2- to 5-membered heteroalkyl, substituted or unsubstituted 5- to 7-membered heterocycloalkyl, substituted or unsubstituted aryl, or substituted or unsubstituted heteroaryl. R¹ may also be selected from H, —OH, —NH₂,

—NO₂, halogen, substituted or unsubstituted (C₁-C₅) alkyl, substituted or unsubstituted 2- to 5-membered heteroalkyl, substituted or unsubstituted 5- to 7-membered heterocycloalkyl, substituted or unsubstituted aryl, or substituted or unsubstituted heteroaryl. In other embodiments, R¹ is H, —NH₂, Br, F, Cl, —CF₃, methyl, —OCH₃, —NH—C(O)—CH₃, —NH—C(O)—CH₂CH₃, or substituted or unsubstituted morpholino.

In another embodiment, R² is —CF₃, Cl, F, —OH, —NH₂, substituted or unsubstituted alkyl, or substituted or unsubstituted heteroalkyl. R² may also be selected from H, Cl, F, —OH, —NH₂, substituted or unsubstituted alkyl, or substituted or unsubstituted heteroalkyl. In other embodiments, R² is selected from H, —OH, —NH₂, substituted or unsubstituted (C₁-C₆) alkyl, and substituted or unsubstituted 2- to 6-membered heteroalkyl. R² may also simply be substituted or unsubstituted (C₁-C₆) alkyl.

Alternatively, R² is H, —OH, —NH₂, methyl, —CF₃, —OCH₃, —OCH(CH₃)₂, —OCH₂CH₂OCH₃, —CH₂C(O)OCH₃, —OCH₂C(O)OCH₃, —C(O)N(CH₃)₂, —CN, —NHC(O)CH₃, or

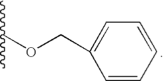

In some embodiments, R³ is H, —OH, —NH₂, —NO₂, —SO₂NH₂, halogen, substituted or unsubstituted alkyl, substituted or unsubstituted heteroalkyl, substituted or unsubstituted 5- to 7-membered cycloalkyl, substituted or unsubstituted 5- to 7-membered heterocycloalkyl, substituted or unsubstituted aryl, or substituted or unsubstituted heteroaryl. R³ may also be substituted or unsubstituted pyrrolyl, substituted or unsubstituted thiazolyl, substituted or unsubstituted pyrrolidinonyl, substituted or unsubstituted pyridinyl, substituted or unsubstituted thiophenyl, substituted or unsubstituted furanyl, substituted or unsubstituted isoquinolinyl, or substituted or unsubstituted dihydroquinolinyl. In other embodiments, R³ is substituted or unsubstituted morpholino, substituted or unsubstituted thiomorpholino, substituted or unsubstituted pyrrolidinyl, substituted or unsubstituted pyrrolidinonyl, substituted or unsubstituted piperidinyl, substituted or unsubstituted piperazinyl, substituted or unsubstituted tetrahydrofuranyl, substituted or unsubstituted tetrahydropyranyl, substituted or unsubstituted tetrahydrothiophenyl, or substituted or unsubstituted tetrahydrothiopyranyl. R³ may also be H, -L¹-OR⁶, -L²-NR⁷R⁸, -L³-CONR⁷R⁸, -L⁴-COOR⁶, or -L⁵-COR⁶. In some embodiments R³ is —NH₂, —NO₂, —SO₂NH₂, Cl, F, I, or Br.

R⁶ may be H, substituted or unsubstituted (C₁-C₆) alkyl, substituted or unsubstituted 2- to 6-membered heteroalkyl, substituted or unsubstituted 5- to 7-membered cycloalkyl, substituted or unsubstituted 5- to 7-membered heterocycloalkyl, substituted or unsubstituted heteroaryl, or substituted or unsubstituted aryl. R⁷ and R⁸ may independently be H, substituted or unsubstituted (C₁-C₆) alkyl, substituted or unsubstituted 2- to 6-membered heteroalkyl, or substituted or unsubstituted heteroaryl.

In some embodiments, R⁶ is H, unsubstituted (C₁-C₄) alkyl, —CH₂CH₂N(CH₃)₂, or substituted or unsubstituted benzyl. R⁷ and R³ may be H, methyl, ethyl, —C(O)CH₃ or substituted or unsubstituted pyridinyl. R⁷ and R⁸ may be joined with the nitrogen to which they are attached to form an unsubstituted pyrrolidinyl. L¹ may be a bond, methylene, ethylene, or propylene. L² may be a bond, methylene, or ethylene. L³ may be a bond. L⁴ may be a bond or ethylene. L¹ may be a bond.

R³ may also be —OCH₃, —OCH₂CH₃,

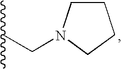

### —C(═O)N(CH₃)₂, —C(═O)OCH₃, —(CH₂)₂C(═O)OCH₂CH₃, —CH₂OH, —(CH₂)₂OH, —(CH₂)₃OH, or —N(CH₃)(CH₂CH₂OCH₃).

In some embodiments, R⁴ and R⁵ are independently selected from H, substituted or unsubstituted alkyl, and substituted or unsubstituted heteroalkyl. In another embodiment, R⁴ and R⁵ are members independently selected from H, substituted or unsubstituted (C₁-C₆) alkyl, substituted or unsubstituted 2- to 6-membered heteroalkyl, and substituted or unsubstituted 5- to 7-membered heteroaryl. In yet another embodiment, R⁴ and R⁵ are members independently selected from H, methyl, —C(O)OC(CH₃)₃, —C(O)CH₃, and pyridinyl. R⁵ may be H.

In some embodiments, A is substituted or unsubstituted thiophenyl, substituted or unsubstituted benzyl, substituted or unsubstituted pyridinyl, substituted or unsubstituted pyrimidinyl, substituted or unsubstituted thiazolyl, substituted or unsubstituted isothiazolyl, substituted or unsubstituted benzimidazolyl, substituted or unsubstituted imidazolyl, substituted or unsubstituted pyrazinyl, substituted or unsubstituted pyridazinyl, substituted or unsubstituted pyrazolyl, or substituted or unsubstituted 1,2,4-oxadiazolyl. A may also be substituted or unsubstituted pyridinyl, substituted or unsubstituted pyrazinyl, substituted or unsubstituted thiazolyl, or substituted or unsubstituted pyrazolyl. Alternatively, A is unsubstituted pyrazinyl, unsubstituted thiazolyl, unsubstituted pyrazolyl, or N-methyl pyrazolyl.

B is selected from substituted or unsubstituted furanyl, substituted or unsubstituted benzyl, substituted or unsubstituted pyridinyl, substituted or unsubstituted 1,2,4-thiadiazolyl, substituted or unsubstituted pyrimidinyl, substituted or unsubstituted pyrazinyl, substituted or unsubstituted thiazolyl, substituted or unsubstituted isoxazolyl, or substituted or unsubstituted pyrazolyl. B may also be substituted or unsubstituted pyridinyl.

In some embodiments, two R³ groups are optionally joined with the atoms to which they are attached to form a substituted or unsubstituted phenyl or substituted or unsubstituted cyclohexanyl. R¹ and R² are optionally joined with the atoms to which they are attached to form a substituted or unsubstituted phenyl or substituted or unsubstituted cyclohexanyl. R² and R⁵ are optionally joined with the atoms to which they are attached to form a substituted or unsubstituted imidazolyl or substituted or unsubstituted morpholino.

In another embodiment, the potassium ion channel modulator has the formula:

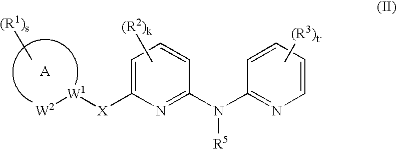

In Formula (II), A is substituted or unsubstituted pyridinyl, substituted or unsubstituted pyrazinyl, substituted or unsubstituted thiazolyl, substituted or unsubstituted pyrimidinyl, substituted or unsubstituted imidazolyl, substituted or unsubstituted benzimidazolyl, or substituted or unsubstituted pyrazolyl. R⁵ is H, substituted or unsubstituted alkyl, substituted or unsubstituted aryl, substituted or unsubstituted heteroaryl, —COR⁶, —COOR⁶, —CONR⁷R⁸, —SO₂R⁶, or —SO₂NR⁷R⁸. X is a bond. R⁶, R⁷, R⁸, s, k, t, W¹ and W² are as defined and described above in the discussion of Formula (I). In some embodiments, A is substituted or unsubstituted thiazolyl.

In another embodiment, the potassium ion channel modulator has the formula:

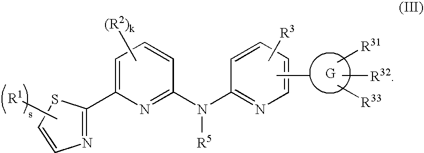

In Formula (III), G is substituted or unsubstituted cyclopropyl, substituted or unsubstituted cyclobutyl, substituted or unsubstituted cyclopentyl, substituted or unsubstituted cyclohexyl, substituted or unsubstituted cycloheptyl, substituted or unsubstituted azetidinyl, substituted or unsubstituted pyrrolidinyl, substituted or unsubstituted piperidinyl, substituted or unsubstituted azepanyl, substituted or unsubstituted piperazinyl, substituted or unsubstituted morpholino, substituted or unsubstituted thiomorpholino, substituted or unsubstituted tetrahydropyridinyl, substituted or unsubstituted diazepanyl, substituted or unsubstituted furanyl, substituted or unsubstituted thienyl, substituted or unsubstituted pyrrolyl, substituted or unsubstituted thiazolyl, substituted or unsubstituted oxazolyl, substituted or unsubstituted pyrazolyl, substituted or unsubstituted oxadiazolyl, substituted or unsubstituted thiadiazolyl, substituted or unsubstituted triazolyl, substituted or unsubstituted tetrazolyl, substituted or unsubstituted phenyl, substituted or unsubstituted pyridinyl, substituted or unsubstituted pyrimidinyl, or substituted or unsubstituted pyrazinyl.

R³ is H, substituted or unsubstituted alkyl, —OR⁶, or halogen. R⁵ is H, substituted or unsubstituted alkyl, substituted or unsubstituted aryl, or substituted or unsubstituted heteroaryl. R³ and R¹² are independently H, substituted or unsubstituted alkyl, —OR³¹¹, —NR³¹²R³¹³—COR³¹¹, —COOR³¹²R³¹³, —CONR³¹²R³¹³, —SO₂R³¹¹, —SO₂NR³¹²R³¹³, oxo, —NO₂, cyano, imino, or halogen. R³³ is H, or substituted or unsubstituted alkyl. R³¹² and R³¹³ are independently H, substituted or unsubstituted alkyl, substituted or unsubstituted aryl, —COR³¹⁴, or —SO₂R³¹⁴. R³¹⁴ is hydrogen, substituted or unsubstituted alkyl, or substituted or unsubstituted heteroalkyl. R³¹¹ is H, substituted or unsubstituted alkyl, or substituted or unsubstituted aryl.

R¹, R², R⁶, s, and k, are as defined and described above in the discussion of Formula (I). Where more than one R³¹¹, R³¹², R³¹³, and/or R³¹⁴ groups are present, each R³¹¹, R³¹², R³¹³, and/or R³¹⁴ group is optionally different.

In another embodiment, the potassium ion channel modulator has the formula:

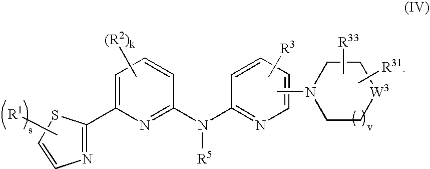

In Formula (IV), W³ is a bond, —O—, —S—, —N(R³²)—, or —C(R³⁴R³⁵)—. The symbol V is an integer from 0 to 2. R³ is H, substituted or unsubstituted alkyl, —OR⁶, or halogen. R⁵ is H, substituted or unsubstituted alkyl, substituted or unsubstituted aryl, or substituted or unsubstituted heteroaryl. R³¹, R³⁴, and R³⁵ are independently H, substituted or unsubstituted alkyl, —OR³¹¹, —NR³¹²R³¹³, —COR³¹¹, —COOR³¹¹, —CONR³¹²R³¹³, oxo, —NO₂, cyano, imino, or halogen. R³² is H, alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, —OR³¹¹, —COR³¹¹, —COOR³¹¹, —CONR³¹²R³¹³, —SO₂R³¹¹, —SO₂NR³¹²R³¹³, oxo, NO₂, cyano, imino, or halogen.

R³³ is H or substituted or unsubstituted alkyl. R³¹² and R³¹³ are independently H, substituted or unsubstituted alkyl, substituted or unsubstituted aryl, —COR³¹⁴, or —SO₂R³¹⁴. R³¹⁴ is hydrogen, substituted or unsubstituted alkyl, or substituted or unsubstituted heteroalkyl. R³¹¹ is H, substituted or unsubstituted alkyl, or substituted or unsubstituted aryl.

R¹, R², R⁶, s, and k, are as defined and described above in the discussion of Formula (I). Where more than one R³¹, R³¹², R³¹³, and/or R³¹⁴ groups are present, each R³¹¹, R³¹², R³¹³, and/or R³¹⁴ group is optionally different.

In another embodiment, the potassium ion channel modulator is selected from (6-Thiazol-2-yl-pyridin-2-yl)-(5-thiophen-3-yl-pyridin-2-yl)-amine, (3-Methoxy-6-thiazol-2-yl-pyridin-2-yl)-[5-(4-methyl-piperazin-1-yl)-pyridin-2-yl]-amine, (5,6,7,8-Tetrahydro-isoquinolin-3-yl)-(6-thiazol-2-yl-pyridin-2-yl)-amine, (3-Methoxy-6-thiazol-2-yl-pyridin-2-yl)-(3,4,5,6-tetrahydro-2H-[1,3′]bipyridinyl-6′-yl)-amine, (3-Methoxy-6-thiazol-2-yl-pyridin-2-yl)-(5-morpholin-4-yl-pyridin-2-yl)-amine, (5-Pyrrolidin-1-ylmethyl-pyridin-2-yl)-(6-thiazol-2-yl-pyridin-2-yl)-amine, 1-{6-[6-(5-Chloro-thiazol-2-yl)-pyridin-2-ylamino]-pyridin-3-yl}-pyrrolidin-2-one, 4-Methyl-1-[6-(6-thiazol-2-yl-pyridin-2-ylamino)-pyridin-3-yl]-piperazin-2-one, [6-(5-Chloro-thiazol-2-yl)-3-methoxy-pyridin-2-yl]-(5-pyrrolidin-1-yl-pyridin-2-yl)-amine, [5-(1,3-Dihydro-isoindol-2-ylmethyl)-pyridin-2-yl]-(6-thiazol-2-yl-pyridin-2-yl)-amine, 1-Methyl-4-[6-(6-thiazol-2-yl-pyridin-2-ylamino)-pyridin-3-yl]-diazepan-5-one, (3-Methoxy-6-thiazol-2-yl-pyridin-2-yl)-(5-pyrrolidin-1-yl-pyridin-2-yl)-amine, (5-Phenyl-pyridin-2-yl)-(6-thiazol-2-yl-pyridin-2-yl)-amine, (5-Bromo-pyridin-2-yl)-[6-(4-methyl-pyrazol-1-yl)-pyridin-2-yl]-amine, (5-Chloro-pyridin-2-yl)-(6-pyrazin-2-yl-pyridin-2-yl)-amine, [5-(3-Fluoro-phenyl)-pyridin-2-yl]-[6-(4-methyl-pyrazol-1-yl)-pyridin-2-yl]-amine, 1-[6-(6-Thiazol-2-yl-pyridin-2-ylamino)-pyridin-3-yl]-piperazin-2-one, 1-[6-(3-Methoxy-6-thiazol-2-yl-pyridin-2-ylamino)-pyridin-3-yl]-pyrrolidin-2-one, and [6-(5-Chloro-thiazol-2-yl)-pyridin-2-yl]-(3,4,5,6-tetrahydro-2H-[1,3′]bipyridinyl-6′-yl)-amine.

In an exemplary embodiment, there is a compound of the structure

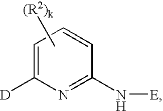

in which k is an integer between 1 and 3. D is selected from substituted or unsubstituted 2-pyridinyl, substituted or unsubstituted 2-pyrimidinyl, substituted or unsubstituted 2-thiazolyl, substituted or unsubstituted imidazolyl, substituted or unsubstituted 1-pyrazolyl, substituted or unsubstituted 2-pyrazinyl. E is selected from substituted or unsubstituted 2-pyridinyl, substituted or unsubstituted 3-pyrazolyl, substituted or unsubstituted 2-thiadiazolyl, substituted or unsubstituted 3-isoxazolyl. R² is selected from H, OH, NH₂, NO₂, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

In some embodiments, each substituted moiety described above for the compounds of the present invention (e.g. the compound of Formulae (I), (II), (III), or (IV)) is substituted with at least one substituent group. The term “substituent group,” as used herein, is defined in detail above in the “Abbreviations and Definitions” section. More specifically, in some embodiments, each substituted alkyl, substituted heteroalkyl, substituted cycloalkyl, substituted heterocycloalkyl, substituted aryl, substituted heteroaryl, substituted alkylene, and/or substituted heteroalkylene, described above are substituted with at least one substituent group. Each substituent group is optionally different. In other embodiments, at least one or all of these groups are substituted with at least one size-limited substituent group. Alternatively, at least one or all of these groups are substituted with at least one lower substituent group. Size-limited substituent groups and lower substituent groups are both defined in detail above in the “Abbreviations and Definitions” section.

In other embodiments, each substituted or unsubstituted alkyl is a substituted or unsubstituted C₁-C₂₀ alkyl, and each substituted or unsubstituted heteroalkyl is a substituted or unsubstituted 2- to 20-membered heteroalkyl.

Alternatively, each substituted or unsubstituted alkyl is a substituted or unsubstituted C₁-C₈ alkyl, and each substituted or unsubstituted heteroalkyl is a substituted or unsubstituted 2- to 8-membered heteroalkyl.

In some embodiments, the present invention provides a polyvalent metal ion (e.g. iron, zinc, copper, cobalt, manganese, and nickel) and a polydentate component of a metal ion chelator. The polydentate component has a structure according to a potassium ion channel modulator described above (e.g. a compound of Formulae (I), (II), (III), or (IV)). The embodiments described above are equally applicable to the present polydentate component of a metal ion chelator. The polyvalent metal ion may be iron, zinc, copper, cobalt, manganese, or nickel.

Also within the scope of the present invention are compounds of the invention that function as poly- or multi-valent species, including, for example, species such as dimers, trimers, tetramers and higher homologs of the compounds of the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or reactive analogues thereof are attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of the invention). Moreover, the framework can be derivatized with a single species of the invention or more than one species of the invention.

In a second aspect, the present invention provides a method for decreasing ion flow through potassium ion channels in a cell, comprising contacting the cell with a potassium ion channel modulating amount of a potassium ion channel modulator described above, including the compounds or Formulae (I), (II), (III), or (IV).

In an exemplary embodiment, the potassium ion channel comprises at least one SK subunit.

In a third aspect, the present invention provides a method for treating a disease through the modulation of potassium ion flow through these channels. The modulators are useful in the treatment of central or peripheral nervous system disorders (e.g., migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases), and as neuroprotective agents (e.g., to prevent stroke and the like). The modulators of the invention are also useful in treating disease states such as gastroesophogeal reflux disorder and gastrointestinal hypomotility disorders, irritable bowel syndrome, secretory diarrhea, asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomi, diabetes type II, hyperinsulinemia, premature labor, baldness, cancer, and immune suppression. This method involves administering, to a patient, an effective amount (i.e. a therapeutically effective amount) of a potassium ion channel modulator described above, including the compounds or Formulae (I), (II), (III), or (IV).

In a fourth aspect, the present invention provides a pharmaceutical composition comprising a pharmaceutically acceptable carrier and a potassium ion channel modulator described above, including the compounds or Formulae (I), (II), (III), or (IV).

Preparation of Potassium Ion Channel Modulators

The following exemplary schemes illustrate methods of preparing the modulators of the present invention. These methods are not limited to producing the compounds shown, but can be used to prepare a variety of modulators such as the compounds and complexes described above. The modulators of the invention can also be produced by methods not explicitly illustrated in the schemes but are well within the skill of one in the art. The modulators can be prepared using readily available starting materials or known intermediates.

In the following schemes, the symbol Y is independently selected from CH₂, N, S, and O. The symbol D is independently selected from -L¹-OR⁶, -L²-NR⁷R⁸, -L³-CONR⁷R⁸, -L⁴-COOR⁶, -L⁵-COR⁶, -L⁶-SO₂R⁶, -L⁷-SO₂NR⁷R⁸, halogen, CN, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. The symbol p is an integer independently selected from 1-5. The symbol q is an integer independently selected from 0-5. R⁶, R⁷, R⁸, L¹, L², L³, L⁴, L⁵, L⁶, and L⁷ are as defined above in the description of the modulators of the present invention.

The substituents of the pyridinyl compounds of the invention can be produced through the methods outlined in Schemes 1-8.

In one embodiment, the substituents of the invention comprise amino-substituted heteroaryl moieties as shown in Schemes 1-6.

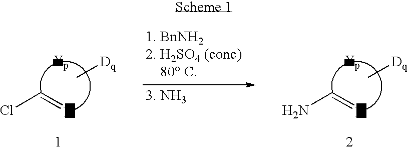

In Scheme 1, compound 1 is reacted with benzylamine, followed by debenzylation in concentrated sulfuric acid, or is directly reacted with ammonia, to produce 2.

An alternative route to producing compound 2 is shown in Scheme 2.

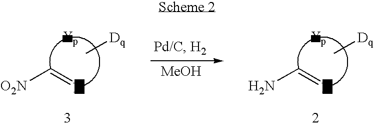

In Scheme 2, a compound 3 is reduced to form compound 2.

Substituents can be added to the amino-substituted heteroaryl moieties as described in Schemes 3-6.

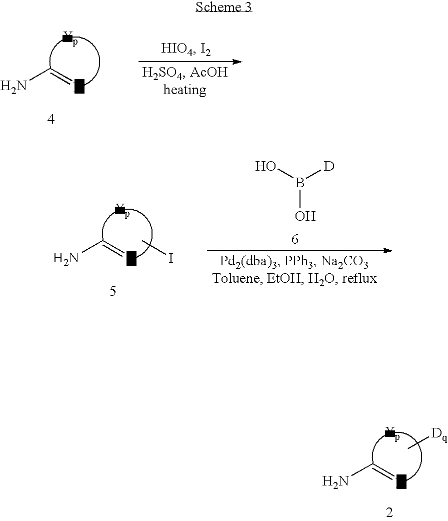

In Scheme 3, compound 4 is iodinated to produce a halosubstituted 2-amino-aza-heterocycle 5. This compound is reacted with a boronic acid 6 in the presence of tris(dibenzylideneacetone)dipalladium(0) (Pd₂(dba)₃), and PPh₃ in toluene, ethanol, and water to produce 2.

In another example, amino substituents can be added to the heteroaryl moieties in the following manner.

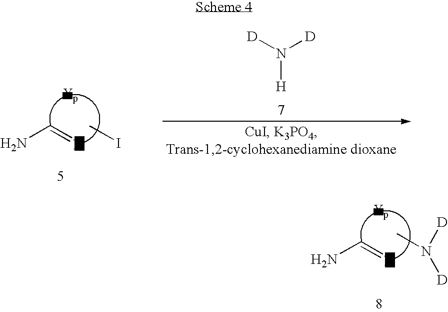

In Scheme 4, an iodo-substituted 2-amino-aza-heterocycle 5 is reacted with an amine 7 or amide using copper catalyzed coupling chemistry to generate a 2-amino-aza-heterocycle 8.

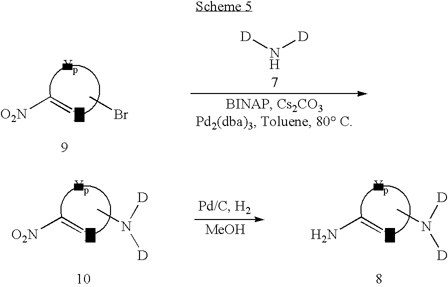

In Scheme 5, a bromo-substituted 2-nitro-aza-heterocycle 9 is reacted with an amine 7 or amide using palladium-catalyzed coupling chemistry to generate an aminosubstituted 2-nitro-aza-heterocycle 10. The nitro adduct is reduced to an amino adduct 8 by a palladium catalyzed hydrogenation.

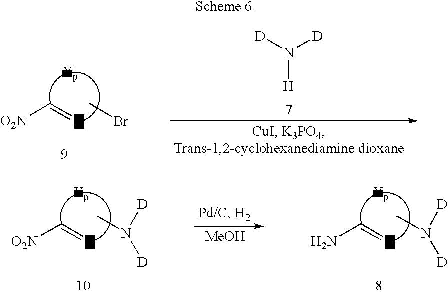

In Scheme 6, a bromo-substituted 2-nitro-aza-heterocycle 9 is reacted with an amine 7 or amide using copper catalyzed coupling chemistry to generate an aminosubstituted 2-nitro-aza-heterocycle 10. The nitro adduct is reduced to an amino adduct 8 by a palladium catalyzed hydrogenation.

In one embodiment, the substituents of the invention comprise halo-substituted heteroaryl moieties as shown in Scheme 7.

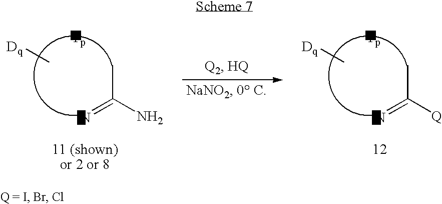

In Scheme 7, compound 11 or 2 or 8 is halogenated by diazotization followed by sodium nitrite in the presence of acid containing halogen at 0° C. to produce compound 12.

In another embodiment, the substituents of the invention comprise stannyl-substituted heteroaryl moieties as shown in Scheme 8.

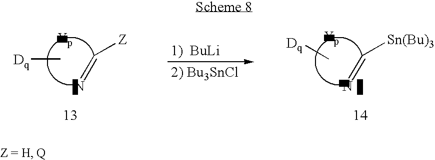

In Scheme 8, compound 13 is stannylated with n-butyllithium to produce compound 14.

A first substituent of the pyridinyl compound can be attached through the methods outlined in Scheme 9 or Scheme 10.

In one embodiment, stannyl-substituted heteroaryl moieties can be attached to the pyridinyl core as shown in Scheme 9.

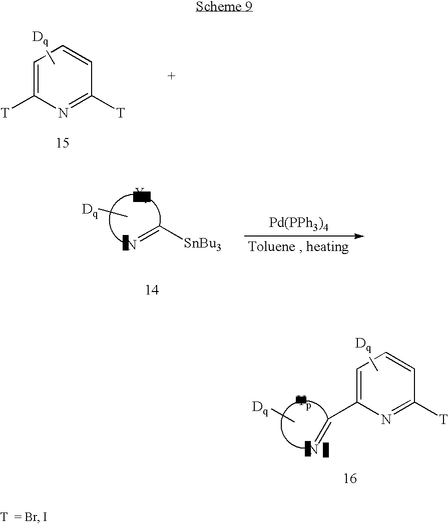

In Scheme 9, addition of compound 14 to a 2,6-dihalopyridine 15 in the presence of a palladium catalyst in toluene produces compound 16.

In another embodiment, halo-substituted heteroaryl moieties can be attached to the pyridinyl core as shown in Scheme 10.

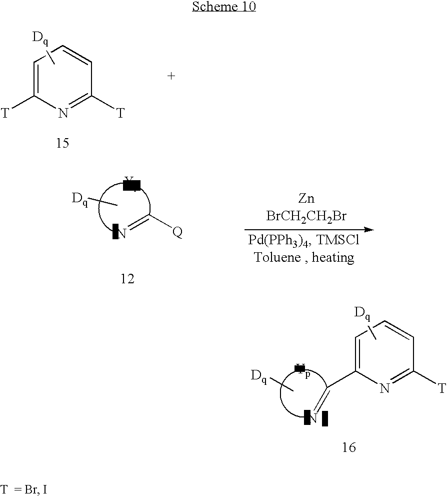

In Scheme 10, addition of compound 12 to a 2,6-dihalopyridine 15 in the presence of zinc dust, dibromoethane, and a palladium catalyst in toluene produces compound 16.

In another embodiment, amino-substituted heteroaryl moieties can be attached to the pyridinyl core as shown in Scheme 11.

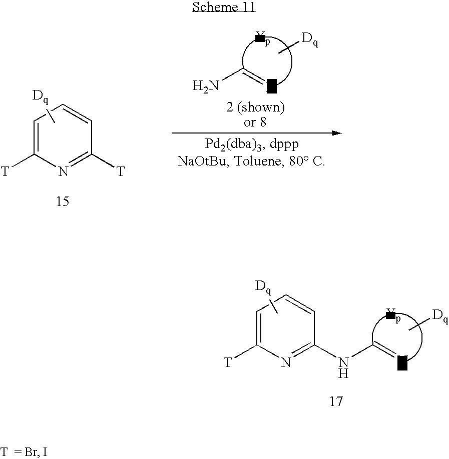

In Scheme 11, addition of compound 2 or 8 to a 2,6-dihalopyridine 15 in the presence of a palladium catalyst in toluene with 1,3-bis(diphenyl phosphino)propane (dppp) produces compound 17.

An alternative method of attaching a first substituent is illustrated in Scheme 12:

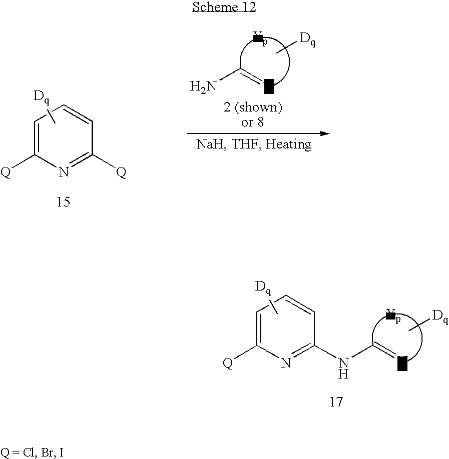

In Scheme 12, addition of compound 2 or 8 to a 2,6-dihalopyridine 15 via sodium hydride in tetrahydrofuran (THF) produces compound 17.

Bis-substituted pyridines are produced from the methods outlined in Scheme 13 or Scheme 14 or Scheme 15.

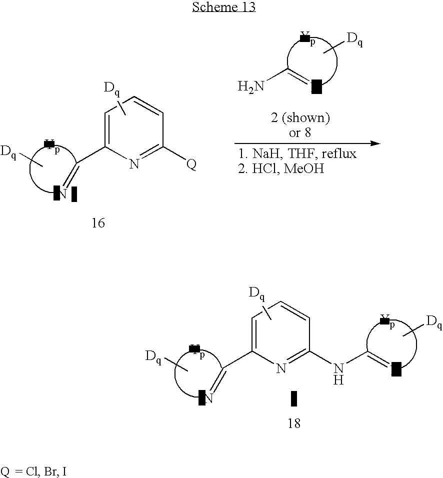

In Scheme 13, compound 2 or 8 is mixed with sodium hydride to facilitate the nucleophilic addition of 2 or 8 to compound 16. A final acid washing step produces a bis-substituted pyridine 18.

Alternative conditions for facilitating this transformation are provided in Scheme 14.

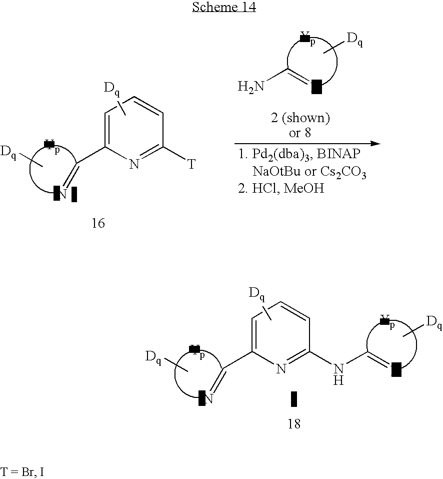

In Scheme 14, addition of compound 2 or 8 to compound 16 in the presence of a palladium catalyst produces compound 18. A final acid washing step produces a bis-substituted pyridine 18.

Compound 18 is alternatively produced as shown in Scheme 15.

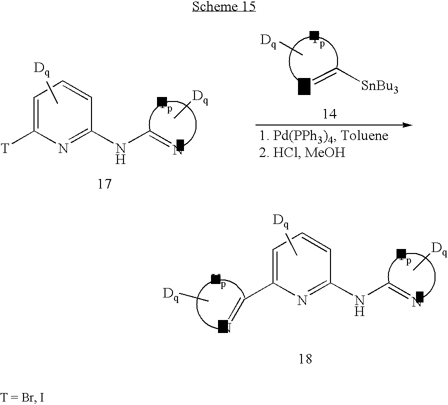

In Scheme 15, compound 14 is added to compound 17 in the presence of a palladium catalyst to form compound 18. A final acid washing step produces a bis-substituted pyridine 18.

An alternative method of creating substituted pyridine compounds is illustrated in Scheme 16:

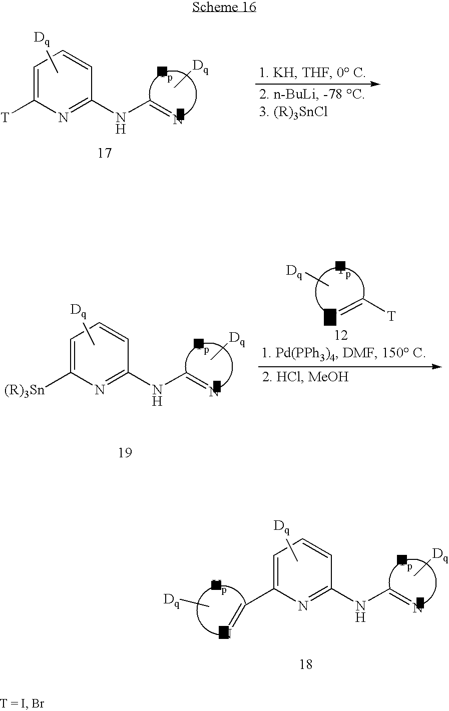

In Scheme 16, compound 17 is first stannylated to produce compound 19. Next, compound 12 is added in the presence of a palladium catalyst to produce the final product 18. A final acid washing step produces a bis-substituted pyridine 18.

Another method of producing the compounds of the invention is exemplified in Scheme 17:

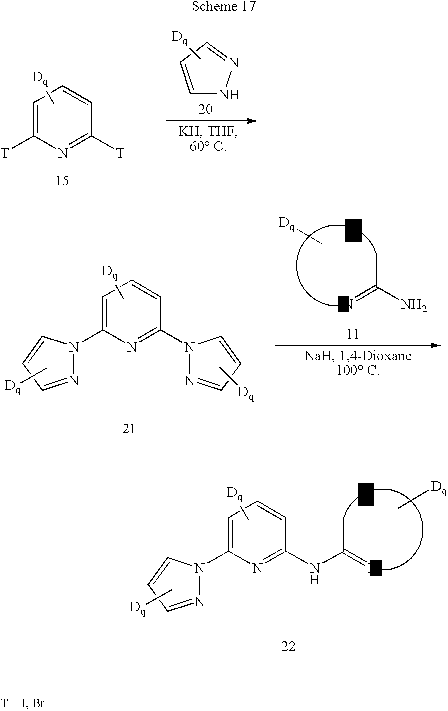

In Scheme 17, compound 15 is mixed with potassium hydride in THF to facilitate the nucleophilic addition of an excess of pyrazole 20 to compound 15 to produce a bis-pyrazoyl pyridine 21. Sodium hydride is then mixed with compound 11 to facilitate the production of compound 22.

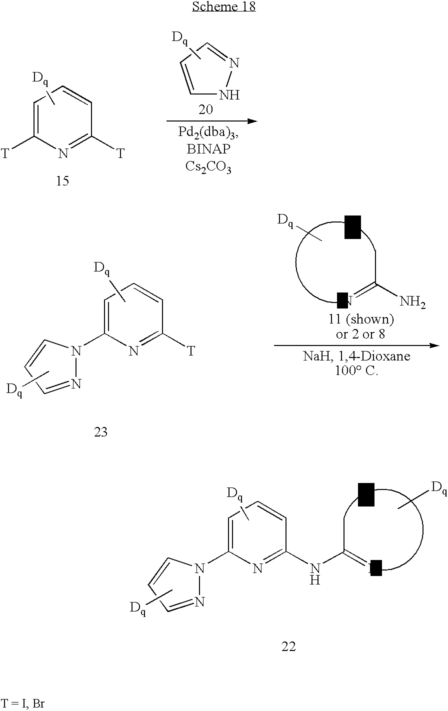

In Scheme 18, 1 equivalent of compound 20 is coupled to compound 15 via palladium catalyzed coupling chemistry to produce mono-pyrazolyl pyridine 23. Sodium hydride is then mixed with compound 11 or 2 or 8 to facilitate its addition to compound 23 and the production of compound 22.

Methods of modifying the pyridinyl compounds of the invention are described in Scheme 19-23.

A method of creating a pyridinyl compounds of the invention with an alcohol substituent is outlined in Scheme 19.

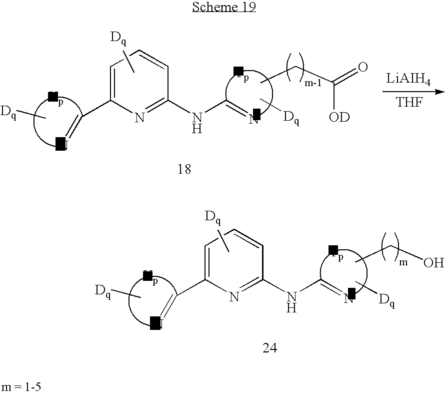

In Scheme 19, compound 18 is reduced to compound 24 through the use of LiAlH₄ in THF.

A method of chlorinating the pyridinyl compounds of the invention is outlined in Scheme 20.

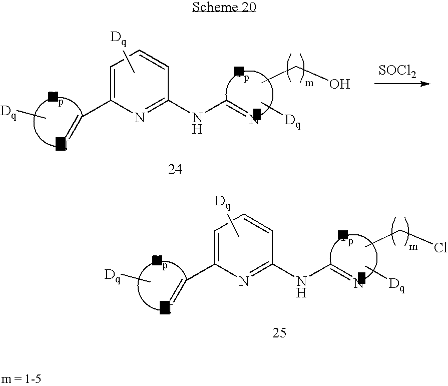

In Scheme 20, compound 24 is converted to compound 25 through the use of SOCl₂.

A method of adding an amine to the pyridinyl compounds of the invention is outlined in Scheme 21.

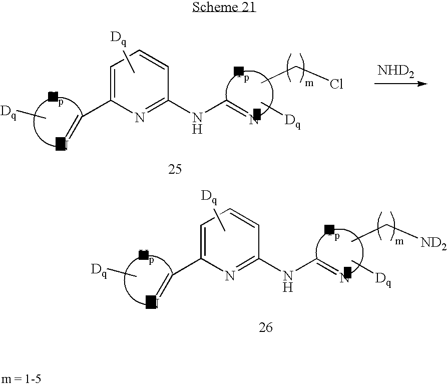

In Scheme 21, compound 25 is reacted with any commercially available primary or secondary amine in order to produce compound 26.

A method of creating a bicyclic pyridinyl compound of the invention is outlined in Scheme 22.

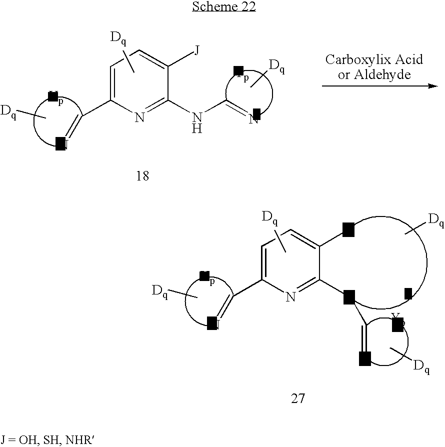

In Scheme 22, compound 18 is reacted with formic acid to produce compound 27. R′ is hydrogen, substituted or unsubstituted alkyl, or substituted or unsubstituted heteroalkyl.

The compounds of the invention also include metal complexes. These metal complexes comprise a polyvalent metal ion and a pyridinyl compound of the invention. In an exemplary embodiment, the polyvalent metal ion can be a transition metal. In another exemplary embodiment, the polyvalent metal ion is a member selected from iron, zinc, copper, cobalt, manganese, and nickel.

A method of creating metal-pyridinyl complexes of the invention is outlined in Scheme 23.

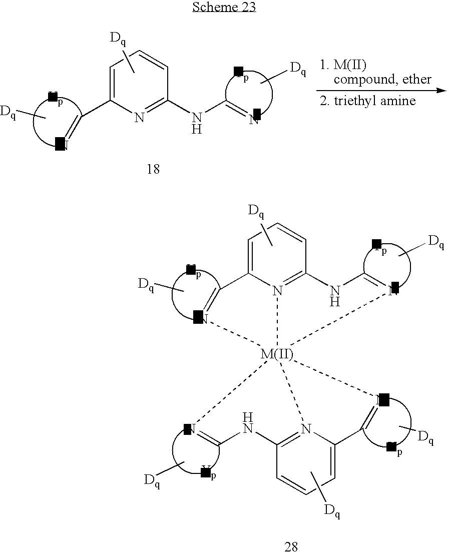

In Scheme 23, compound 18 is first mixed with FeClO₄ in ether. To this mixture is added triethylamine which then forms metal complex 28.

### III. Assays for Modulators of Potassium Ion Channels

SK monomers as well as SK alleles and polymorphic variants are subunits of potassium ion channels. The activity of a potassium ion channel comprising SK subunits can be assessed using a variety of in vitro and in vivo assays, e.g., measuring current, measuring membrane potential, measuring ion flow, e.g., potassium or rubidium, measuring potassium concentration, measuring second messengers and transcription levels, using potassium-dependent yeast growth assays, and using e.g., voltage-sensitive dyes, radioactive tracers, and patch-clamp electrophysiology.

Furthermore, such assays can be used to test for inhibitors and activators of channels comprising SK. The SK family of channels is implicated in a number of disorders that are targets for a therapeutic or prophylactic regimen, which functions by blockade or inhibition of one or more members of the SK channel family. The modulators and methods of the invention are useful to treat central or peripheral nervous system disorders (e.g., migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases), and as neuroprotective agents (e.g., to prevent stroke and the like). The modulators of the invention are also useful in treating disease states such as gastroesophogeal reflux disorder and gastrointestinal hypomotility disorders, irritable bowel syndrome, secretory diarrhea, asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomi, diabetes type II, hyperinsulinemia, premature labor, baldness, cancer, and immune suppression.

Modulators of the potassium ion channels are tested using biologically active SK, either recombinant or naturally occurring, or by using native cells, like cells from the nervous system expressing an SK channel. SK channels can be isolated, co-expressed or expressed in a cell, or expressed in a membrane derived from a cell. In such assays, SK is expressed alone to form a homomeric potassium ion channel or is co-expressed with a second subunit (e.g., another SK family member) so as to form a heteromeric potassium ion channel. Modulation is tested using one of the in vitro or in vivo assays described above. Samples or assays that are treated with a potential potassium ion channel inhibitor or activator are compared to control samples without the test modulator, to examine the extent of modulation. Control samples (untreated with activators or inhibitors) are assigned a relative potassium ion channel activity value of 100. Inhibition of channels comprising SK is achieved when the potassium ion channel activity value relative to the control is less than 70%, preferably less than 40% and still more preferably, less than 30%. Modulators that decrease the flow of ions will cause a detectable decrease in the ion current density by decreasing the probability of a channel comprising SK being open, by decreasing conductance through the channel, and decreasing the number or expression of channels.

Changes in ion flow may be assessed by determining changes in polarization (i.e., electrical potential) of the cell or membrane expressing the potassium ion channel. A preferred means to determine changes in cellular polarization is by measuring changes in current or voltage with the voltage-clamp and patch-clamp techniques, using the “cell-attached” mode, the “inside-out” mode, the “outside-out” mode, the “perforated cell” mode, the “one or two electrode” mode, or the “whole cell” mode (see, e.g., Ackerman et al., New Engl. J. Med. 336: 1575-1595 (1997)). Whole cell currents are conveniently determined using the standard methodology (see, e.g., Hamil et al., Pflugers. Archiv. 391: 85 (1981)). Other known assays include: radiolabeled rubidium flux assays and fluorescence assays using voltage-sensitive dyes (see, e.g., Vestergarrd-Bogind et al., J. Membrane Biol. 88: 67-75 (1988); Daniel et al., J. Pharmacol. Meth. 25: 185-193 (1991); Holevinsky et al., J. Membrane Biology 137: 59-70 (1994)). Assays for modulators capable of inhibiting or increasing potassium flow through the channel proteins can be performed by application of the modulators to a bath solution in contact with and comprising cells having a channel of the present invention (see, e.g., Blatz et al., Nature 323: 718-720 (1986); Park, J. Physiol. 481: 555-570 (1994)). Generally, the modulators to be tested are present in the range from about 1 pM to about 100 in M, preferably from about 1 μM to about 1 μM.

The effects of the test modulators upon the function of the channels can be measured by changes in the electrical currents or ionic flow or by the consequences of changes in currents and flow. Changes in electrical current or ionic flow are measured by either increases or decreases in flow of ions such as potassium or rubidium ions. The cations can be measured in a variety of standard ways. They can be measured directly by concentration changes of the ions or indirectly by membrane potential or by radio-labeling of the ions. Consequences of the test modulator on ion flow can be quite varied. Accordingly, any suitable physiological change can be used to assess the influence of a test modulator on the channels of this invention. The effects of a test modulator can be measured by a toxin-binding assay. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immunoresponses (e.g., T cell activation), changes in cell metabolism such as cell growth or pH changes, and changes in intracellular second messengers such as calcium, or cyclic nucleotides.

### IV. Pharmaceutical Compositions for Use as Potassium Ion Channel Modulators

In another aspect, the present invention provides pharmaceutical compositions comprising a pharmaceutically acceptable carrier and a potassium ion channel modulator described above, including the compounds or Formulae (I), (II), (III), or (IV).

Formulation of the Modulators

The modulators of the present invention can be prepared and administered in a wide variety of oral, parenteral and topical dosage forms. Thus, the modulators of the present invention can be administered by injection, that is, intravenously, intramuscularly, intracutaneously, subcutaneously, intraduodenally, or intraperitoneally. Also, the modulators described herein can be administered by inhalation, for example, intranasally. Additionally, the modulators of the present invention can be administered transdermally. Accordingly, the present invention also provides pharmaceutical compositions comprising a pharmaceutically acceptable carrier and either a modulator, or a pharmaceutically acceptable salt of a modulator.

For preparing pharmaceutical compositions from the modulators of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active modulator. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active modulator with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

### V. Methods for Decreasing Ion Flow in Potassium Ion Channels

In yet another aspect, the present invention provides a method for decreasing ion flow through potassium ion channels in a cell, comprising contacting the cell with a potassium ion channel modulating amount of a potassium ion channel modulator described above, including the compounds or Formulae (I), (II), (III), or (IV).

In an exemplary embodiment, the potassium ion channels comprise at least one SK subunit.

The methods provided in this aspect of the invention are useful in the therapy of conditions mediated through potassium ion flow, as well as for the diagnosis of conditions that can be treated by decreasing ion flow through potassium ion channels. Additionally the methods are useful for determining if a patient will be responsive to therapeutic agents which act by modulating potassium ion channels. In particular, a patient's cell sample can be obtained and contacted with a potassium ion channel modulator described above (e.g. a compound of Formulae (I), (II), (III), or (IV)) and the ion flow can be measured relative to a cell's ion flow in the absence of the modulator. A decrease in ion flow will typically indicate that the patient will be responsive to a therapeutic regiment of the modulator.

### VI. Methods for Treating Conditions Mediated by Potassium Ion Channels

In still another aspect, the present invention provides a method for treating a disease through the modulation of potassium ion flow through potassium ion channels. The modulation may be activation or inhibition of the potassium ion flow. Thus, the modulators of the present invention may be inhibitors of potassium ion flow through potassium ion channels (i.e. decrease the flow relative to the absence of the modulator) or activators of potassium ion flow through potassium ion channels (i.e. increase the flow relative to the absence of the modulator).

The modulators are useful in the treatment of central or peripheral nervous system disorders (e.g., migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases), and as neuroprotective agents (e.g., to prevent stroke and the like). The modulators of the invention are also useful in treating disease states such as gastroesophogeal reflux disorder and gastrointestinal hypomotility disorders, irritable bowel syndrome, secretory diarrhea, asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomi, diabetes type II, hyperinsulinemia, premature labor, baldness, cancer, and immune suppression. This method involves administering, to a patient, an effective amount (e.g. a therapeutically effective amount) of a modulator of the present invention (a compound or complex of the present invention).

Thus, the present invention provides a method of decreasing ion flow through potassium ion channels in a cell. The method includes contacting the cell with a potassium ion channel-modulating amount of a modulator of the present invention. In some embodiments, the potassium ion channel includes at least one SK subunit. The cell may be isolated or form part of a organ or organism.

The modulators provided herein find therapeutic utility via modulation of potassium ion channels in the treatment of diseases or conditions. The potassium ion channels that are typically modulated are described herein. As noted above, these channels may include homomultimers and heteromultimers.

In therapeutic use for the treatment of neurological conditions, the modulators utilized in the pharmaceutical method of the invention are administered at the initial dosage of about 0.001 mg/kg to about 1000 mg/kg daily. A daily dose range of about 0.1 mg/kg to about 100 mg/kg is more typical. The dosages, however, may be varied depending upon the requirements of the patient, the severity of the condition being treated, and the modulator being employed. Determination of the proper dosage for a particular situation is within the skill of the practitioner. Generally, treatment is initiated with smaller dosages, which are less than the optimum dose of the modulator. Thereafter, the dosage is increased by small increments until the optimum effect under the circumstances is reached. For convenience, the total daily dosage may be divided and administered in portions during the day.

The materials and methods of the present invention are further illustrated by the examples which follow. These examples are offered to illustrate, but not to limit, the claimed invention.

### EXAMPLES

### General

In the examples below, unless otherwise stated, temperatures are given in degrees Celsius (° C.); operations were carried out at room or ambient temperature, “rt,” or “RT,” (typically a range of from about 18-25° C.; evaporation of solvent was carried out using a rotary evaporator under reduced pressure (typically, 4.5-30 mm Hg) with a bath temperature of up to 60° C.; the course of reactions was typically followed by thin layer chromatography (TLC) and reaction times are provided for illustration only; melting points are uncorrected; products exhibited satisfactory ¹H-NMR and/or microanalytical data; yields are provided for illustration only; and the following conventional abbreviations are also used: mp (melting point), L (liter(s)), mL (milliliters), mmol (millimoles), g (grams), mg (milligrams), min (minutes), and h (hours).

Unless otherwise specified, all solvents (HPLC grade) and reagents were purchased from suppliers and used without further purification. Reactions were conducted under a blanket of argon unless otherwise stated. Analytical TLC was performed on Whatman Inc. 60 silica gel plates (0.25 mm thickness). Compounds were visualized under UV lamp (254 nM) or by developing with KMnO₄/KOH, ninhydrin or Hanessian's solution. Flash chromatography was done using silica gel from Selectro Scientific (particle size 32-63). ¹H NMR, ¹⁹F NMR and ¹³C NMR spectra were recorded on a Varian 300 machine at 300 MHz, 282 MHz and 75.7 MHz, respectively. Molecular weight (M+1) determinations were performed using ES+ or FAB+ ionization on Sciex, Micromass, or JEOL mass spectrometers. Melting points were recorded on a Electrothermal IA9100 apparatus and were uncorrected.

### Example 1

### Preparation of 2 from 1

1.1 Nucleophilic Replacement

A mixture of 14.7 mmol of 1 and 75 mmol of benzylamine was heated at 220° C. for 6 h in a sealed tube. The reaction mixture was concentrated it vacuo and the residue was purified by column chromatography on silica gel to give 7.0 mmol of N-benzyl pyridine-2-amine.

A solution of 6.9 mmol of N-benzyl pyridin-2-amine in 15 mL of conc. H₂SO₄ was stirred at 80° C. for 1 h. The reaction mixture was poured into crushed ice and neutralized with 28% NH₄OH. The mixture was extracted with AcOEt and the organic phase was washed with brine, dried over MgSO₄, and concentrated in vacuo. The residue was purified by column chromatography on silica gel to give 5.0 mmol of 2.

1.2 Results

Analytical data for exemplary compounds of structure 2 are provided below.

1.2.a 5-Hexylpyridin-2-ylamine

¹H NMR (300 MHz, CDCl₃) δ 7.88 (d, J=2.2 Hz, 1H), 7.26 (dd, J₁=8.4 Hz, J₂=2.2 Hz, 1H), 6.45 (d, J=8.4 Hz, 1H), 4.27 (br s, 2H), 2.45 (d, J=6.6 Hz, 1H), 1.48-1.56 (m, 2H), 1.27-1.35 (m, 6H), 0.88 (t, J=6.6 Hz, 3H); MS m/z: 178 (M+1).

1.2.b 5-tert-Butylpyridin-2-ylamine

¹H NMR (300 MHz, CDCl₃) δ 8.08 (d, J=2.6 Hz, 1H), 7.47 (dd, J₁=8.6 Hz, J₂=2.6 Hz, 1H), 6.47 (dd, J₁=8.6 Hz, J₂=0.7 Hz, 1H), 1.28 (s, 9H); MS m/z: 151 (M+1).

### Example 1A

### Preparation of 5-[2-(Benzyloxy)ethyl]pyridin-2-amine

A solution of 20.2 mmol of 5-[2-(benzyloxy)ethyl]-2-chloropyridine in saturated NH₃/MeOH (100 ml) was stirred at 260° C. for 8 h in a sealed tube. The reaction mixture was concentrated in vacuo and the residue was purified by column chromatography on silica gel to give 4.7 mmol of 5-[2-(benzyloxy)ethyl]pyridin-2-amine.

¹H NMR (300 MHz, CDCl₃) δ 7.94 (d, J=1.8 Hz, 1H), 7.25-7.37 (m, 6H), 6.45 (dd, J₁=8.4 Hz, J₂=0.7 Hz, 1H), 4.51 (s, 2H), 4.31 (br s, 2H), 3.62 (t, J=6.9 Hz, 2H), 2.78 (t, J=6.9 Hz, 2H); MS m/z: 228 (M+1).

### Example 2

### Preparation of 2 from 3

2.1 Catalytic Reduction

A solution or a suspension of 15 mmol of 3 and 0.5 g of Pd/C (10%) in 150 mL of methanol was stirred overnight under H2 (1 atm). After filtering through celite, the solution was concentrated under a reduced pressure to give 15 mmol of 2.

### Example 3

### Preparation of 2

3.1 Iodination of 4

A mixture of 240 mmol of 4, 58 mmol of HIO₄, and 240 mmol of I₂ in 60 mL of water, 4 mL of concentrated H₂SO₄, and 200 mL of acetic acid was stirred at 80° C. for 4 h. Excess I₂ was neutralized by the addition of 200 mL of saturated Na₂S₂O₃ solution. The resulting aqueous solution was extracted with EtOAc. The organic phase was washed with saturated NaCl, dried over MgSO₄, and concentrated under a reduced pressure. The residue was purified by column chromatography on silica gel to give 136 mmol of 5.

3.2 Suzuki Cross Coupling

A mixture of 15 mmol of 5, 15 mmol of 6, 0.35 mmol of Pd₂(dba)₃, and 2.4 mmol of PPh₃ in 40 mL of toluene, 20 mL of ethanol, and 20 mL of water was refluxed overnight under N₂. The reaction mixture was diluted with 300 mL of ethyl acetate and the organic solution was washed with saturated NaCl, dried over MgSO₄, and concentrated under a reduced pressure. The residue was purified by column chromatography on silica gel to give 13.1 mmol of 2.

3.3 Results

Analytical data for exemplary compounds of structure 2 are provided below.

3.3.a 5-(2-Methoxy-phenyl)-pyridin-2-ylamine

¹H NMR (300 MHz, DMSO-d₆) δ 7.99 (d, J=2.0 Hz, 1H), 7.48 (dd, J₁=8.6 Hz, J₂=2.3 Hz, 1H), 7.26 (d, J=7.5 Hz, 1H), 7.21 (d, J=6.1 Hz, 1H), 7.03 (d, J=8.0 Hz, 1H), 6.96 (t, J=7.3 Hz, 1H), 6.44 (d, J=8.5 Hz, 1H), 5.94 (s, 2H), 3.73 (s, 3H); MS m/z: 201 (M+1).

3.3.b (5-Methyl-furan-2-yl)-pyridin-2-ylamine

¹H NMR (300 MHz, DMSO-d₆) δ 8.17 (d, J=2.0 Hz, 1H), 7.63-7.52 (m, 2H), 6.48 (d, J=3.2 Hz, 1H), 6.43 (d, J=8.7 Hz, 1H), 6.08 (s, 2H), 2.27 (s, 3H); MS m/z: 175 (M+1).

3.3.c [3,3′]Bipyridinyl-6-ylamine

¹H NMR (300 MHz, DMSO-d₆) δ 8.78 (d, J=2.1 Hz, 1H), 8.44 (dd, J₁=4.9 Hz, J₂=1.6 Hz, 1H), 8.27 (d, J=2.2 Hz, 1H), 7.94 (dt, J=8.0 Hz, J₂=1.9 Hz, 1H), 7.73 (dd, J₁=8.7 Hz, J₂=2.6 Hz, 1H), 7.38 (dd, J₃=8.7 Hz, J₂=2.6 Hz, 1H), 6.52 (d, J=8.7 Hz, 1H), 6.17 (s, 2H); MS m/z: 172 (M+1).

3.3.d 5-(4-Fluoro-phenyl)-4-methyl-pyridin-2-ylamine

¹H NMR (300 MHz, DMSO-d₆) δ 7.68 (s, 1H), 7.30 (dd, J₃=8.5 Hz, J₂=5.7 Hz, 2H), 7.19 (t, J=8.9 Hz, 2H), 6.33 (s, 1H), 5.87 (s, 2H), 2.07 (s, 3H); MS m/z: 203 (M+1).

3.3.e 5-(3-Fluoro-phenyl)-pyridin-2-ylamine

¹H NMR (300 MHz, DMSO-d₆) δ 8.27 (d, J=2.3 Hz, 1H), 7.71 (d, J=8.6 Hz, 1H), 7.42-7.38 (m, 3H), 7.08-7.01 (m, 1H), 6.49 (d, J=8.6 Hz, 1H), 6.15 (s, 2H); MS m/z: 189

(M+1).

3.3f 5-Thiophen-2-yl-pyridin-2-ylamine

¹H NMR (300 MHz, DMSO-d₆) δ 8.19 (d, J=2.3 Hz, 1H), 7.61 (d, J=8.5 Hz, 1H), 7.37 (d, J=5.1 Hz, 1H), 7.25 (d, J=3.3 Hz, 1H), 7.04 (t, J=4.7 Hz, 1H), 6.45 (d, J=8.7 Hz, 1H), 6.14 (s, 2H); MS m/z: 177 (M+1).

3.3f 2,3′-Bipyridin-6′-amine

¹H NMR (300 MHz, CDCl₃) δ 8.69 (d, J=7.0 Hz, 1H), 8.62-8.66 (m, 1H), 8.12 (ddd, J=0.9 Hz, 2.4 Hz, 8.6 Hz, 1H), 7.67-7.74 (m, 1H), 7.60-7.64 (m, 1H), 7.14-7.20 (m, 1H), 6.59 (dt, J=0.8 Hz, 8.6 Hz, 1H); MS m/z: 172 (M+1)

### Example 4

### Preparation of 8 from 5

4.1 Ullmann Cross-Coupling

To a solution of 50.0 mmol of 5 and 60.0 mmol of 7 in 50.0 mL of 1,4-dioxane was added 0.500 mmol of copper (I) iodide followed by the addition of 100 mmol of K₃PO₄ and 5 mmol of trans-cyclohexanediamine, then the resulting mixture was stirred at 100° C. for 16 h. The reaction mixture was cooled to room temperature and diluted with 500 mL of H₂O.

The resulting aqueous solution was extracted with CHCl₃. The organic phase was washed with saturated NaCl, dried over MgSO₄ and concentrated in vacuo. The crude product was purified by column chromatography to give 43.4 mmol of 8.

4.2 Results

Analytical data for exemplary compounds of structure 8 are provided below.

4.2.a tert-Butyl 4-(6-aminopyridin-3-yl)-3-oxopiperazine-1-carboxylate

¹H NMR (400 MHz, CDCl₃) δ 7.97-8.00 (m, 1H), 7.35-7.40 (m, 1H), 6.50-6.54 (m, 1H), 4.54 (br s, 2H), 4.24 (s, 2H), 3.65-3.69 (m, 2H), 3.75-3.80 (m, 2H), 1.50 (s, 9H); MS m/z: 293 (M+1).

4.2.b 5-(4-Methyl-1,4-diazepan-1-yl)pyridin-2-ylamine

¹H NMR (400 MHz, DMSO-d₆) δ 7.46 (d, J=3.5 Hz, 1H), 6.95 (dd, J=8.8 Hz, J₂=3.5 Hz, 1H), 6.38 (d, J=8.8 Hz, 1H), 5.04 (br s, 2H), 3.26-3.40 (m, 4H), 2.53-2.59 (m, 2H), 2.41-2.47 (m, 2H), 2.24 (s, 3H), 1.78-1.90 (m, 2H); MS m/z: 207 (M+1).

4.2.c 4-(6-Aminopyridin-3-yl)-1-methyl-1,4-diazepain-5-one

¹H NMR (400 MHz, DMSO-d₆) δ 7.71 (d, J=2.9 Hz, 1H), 7.18 (dd, J₁=8.8 Hz, J₂=2.9 Hz, 1H), 6.41 (d, J=8.8 Hz, 1H), 5.90 (br s, 2H), 3.64-3.71 (m, 2H), 2.51-2.62 (m, 4H), 2.26 (s, 3H); MS m/z: 221(M+1).

4.2.d tert-Butyl 4-(6-aminopyridin-3-yl)-5-oxo-1,4-diazepane-1-carboxylate

¹H NMR (400 MHz, CDCl₃) δ 7.90 (d, J=2.8 Hz, 1H), 7.29 (dd, J₁=8.8 Hz, J₂=2.8 Hz, 1H), 6.50 (d, J=8.8 Hz, 1H), 4.54 (br s, 2H), 3.71-3.75 (m, 6H), 2.80-2.83 (m, 2H), 1.49 (s, 9H); MS m/z: 307 (+1).

### Example 5

### Preparation of 8

5.1 Buchwald Cross-Coupling

A mixture of 30 mmol of 9, 30 mmol of 7, 0.04 mmol of Pd₂(dba)₃, 0.08 mmol of rac-2,2′-bis(phenylphosphino)-1,1′-binaphthyl (BINAP), and 42 mmol of Cs₂CO₃ in 100 mL of dry toluene was stirred at 80° C. for two days under N₂. The reaction mixture was diluted with 400 mL of ethyl acetate and the organic solution was washed with saturated NaCl, dried over MgSO₄, and concentrated under reduced pressure. The residue was crystallized in ethyl acetate to yield 15.8 mmol of 10.

A solution or a suspension of 15 mmol of 10 and 0.5 g of Pd/C (10%) in 150 mL of methanol was stirred overnight under H₂ (1 atm). After filtering through celite, the solution was concentrated under a reduced pressure to give 15 mmol of 8.

5.2 Results

Analytical data for exemplary compounds of structure 8 are provided below.

5.2.a 5-(4-Methyl-piperazin-1-yl)-pyridin-2-ylamine

¹H NMR (300 MHz, DMSO-d₆) δ 7.56 (d, J=2.7 Hz, 1H), 7.13 (dd, J₁=8.9 Hz, J₂=2.9 Hz, 1H), 6.36 (d, J=8.8 Hz, 1H), 5.36 (s, 2H), 2.89 (t, J=5.0 Hz, 4H), 2.40 (t, J=5.0 Hz, 4H), 2.18 (s, 3H); MS m/z: 193 (M+1).

5.2.b 4-Methyl-3,4,5,6-tetrahydro-2H-[1,3′]bipyridinyl-6′-ylainine

¹H NMR (300 MHz, DMSO-d₆) δ 7.56 (d, J=2.8 Hz, 1H), 7.11 (dd, J₁=8.9 Hz, J₂=3.0 Hz, 1H), 6.35 (d, J=8.8 Hz, 1H), 5.34 (s, 2H), 3.26 (d, J=12.0 Hz, 2H), 2.45 (dt, J₁=9.3 Hz, J₂=4.2 Hz, 2H), 1.64 (d, J=12.5 Hz, 2H), 1.4-1.3 (m, 1H), 1.44-1.28 (m, 2H), 0.90 (d, J=6.5 Hz, 3H); MS m/z: 192 (M+1).

5.2.c 1-(6-Aminopyridin-3-yl)-pyrrolidin-2-one

¹H NMR (300 MHz, DMSO-d₆) δ 8.03 (d, J=2.6 Hz, 1H), 7.63 (dd, J₁=8.9 Hz, J₂=2.6 Hz, 1H), 6.42 (d, J=8.9 Hz, 1H), 5.83 (s, 2H), 3.70 (t, J=7.0 Hz, 2H), 2.39 (t, J=7.8 Hz, 2H), 2.01 (dd, J₁=7.1 Hz, J₂=7.9 Hz, 2H); MS m/z: 178 (M+1).

5.2.d 1-(6-Aminopyridin-3-yl)piperidin-2-one

¹H NMR (400 MHz, DMSO-d₆) δ 7.76 (d, J=2.4 Hz, 1H), 7.24 (dd, J₁=8.8 Hz, J₂=2.4 Hz, 1H), 6.42 (d, J=8.8 Hz, 1H), 5.90 (br s, 2H), 3.49 (t, J=6.0 Hz, 2H), 2.34 (t, J=6.0 Hz, 2H), 1.77-1.85 (m, 4H); MS m/z: 192 (N+1).

5.2.e 1-(6-Aminopyridin-3-yl)piperidin-4-ol

¹H NMR (400 MHz, DMSO-d₆) δ 7.59 (d, J=2.4 Hz, 1H), 7.14 (dd, J₁=9.2 Hz, J₂=2.4 Hz, 2H), 6.38 (d, J=9.2 Hz, 1H), 5.34 (br s, 2H), 4.63 (1H, d, J=4.4 Hz), 3.50-3.57 (m, 1H), 3.18-3.23 (m, 2H), 2.59-2.65 (m, 2H), 1.76-1.83 (m, 2H), 1.44-1.54 (m, 2H); MS m/z: 194 (M+1).

5.2.f 5-Piperidin-1-ylpyridin-2-ylamine

¹H NMR (400 MHz, CDCl₃) δ 7.79 (d, J=2.8 Hz, 1H), 7.17 (dd, J₁=8.8 Hz, J₂=2.8 Hz, 1H), 6.47 (dd, J₁=8.0 Hz, J₂=0.8 Hz, 1H), 4.11 (br s, 2H), 2.98 (d, J=5.2 Hz, 2H), 2.97 (d, J=5.2 Hz, 2H), 1.68-1.74 (m, 4H), 1.51-1.57 (m, 2H); MS m/z: 178 (M+1).

5.2.g 5-(4-Isopropylpiperazin-1-yl)pyridin-2-ylamine

¹H NMR (300 MHz, DMSO-d₆) δ 7.55-7.60 (m, 1H), 7.10-7.17 (m, 1H), 6.35-6.42 (m, 1H), 5.34 (br s, 2H), 2.85-2.94 (m, 4H), 2.50-2.70 (m, 5H), 0.95-1.02 (m, 6H); MS m/z: 221 (M+1).

5.2.h tert-Butyl 4-(6-aminopyridin-3-yl)piperazine-1-carboxylate

¹H NMR (400 MHz, CDCl₃) δ 7.78 (d, J=2.8 Hz, 1H), 7.17 (dd, J₁=8.8 Hz, J₂=2.8 Hz, 1H), 6.49 (d, J=8.8 Hz, 1H), 4.21 (br s, 2H), 3.57 (t, J=5.2 Hz, 4H), 2.96 (t, J=5.2 Hz, 4H), 1.48 (s, 9H); MS m/z: 279 (M+1).

5.2.i 1-(6-Aminopyridin-3-yl)-4-methylpiperazin-2-one

¹H NMR (300 MHz, DMSO-d₆) δ 7.80 (d, J=2.4 Hz, 1H), 7.28 (dd, J₁=8.7 Hz, J₂=2.7 Hz, 1H), 6.43 (d, J=8.8 Hz, 1H), 5.97 (br s, 2H), 3.53 (t, J=5.4 Hz, 2H), 3.06 (s, 2H), 2.68 (t, J=5.4 Hz, 2H), 2.26 (s, 3H); MS m/z: 207 (M+1).

5.2j 5-[3-(Dimethylamino)pyrrolidin-1-yl]pyridin-2-ylamine

¹H NMR (400 MHz, CDCl₃) δ 7.78 (d, J=2.8 Hz, 1H), 6.83 (dd, J₁=8.8 Hz, J₂=2.8 Hz, 1H), 6.49 (d, J=8.8 Hz, 1H), 3.96 (br s, 2H), 3.24-3.41 (m, 3H), 3.09 (t, J=8.0 Hz, 1H), 2.82-2.90 (m, 1H), 2.35 (s, 6H), 2.14-2.22 (m, 1H), 1.86-1.96 (m, 1H); MS m/z: 206 (M+1).

5.2.k N⁵-1-Azabicyclo[2.2.2]oct-3-ylpyridin-2,5-yldiamine

¹H NMR (400 MHz, CDCl₃) δ 7.56 (d, J=2.8 Hz, 1H), 6.86 (dd, J₁=8.4 Hz, J₂=2.8 Hz, 1H), 6.44 (d, J=8.4 Hz, 1H), 4.00 (br s, 2H), 3.34-3.37 (m, 1H), 2.80-2.90 (m, 4H), 2.50-2.53 (m, 1H), 1.23-1.97 (m, 6H); MS m/z: 218 (M+1).

5.2.l 5-(2,4,5-Trimethylpiperazin-1-yl)pyridin-2-ylainine

¹H NMR (400 MHz, CDCl₃) δ 7.91 (d, J=2.8 Hz, 1H), 7.30 (dd, J₁=8.8 Hz, J₂=2.8 Hz, 1H), 6.49 (d, J=8.8 Hz, 1H), 4.29 (br s, 2H), 3.06 (m, 1H), 2.86 (dd, J₁=11.2 Hz, J₂=3.2 Hz, 2H), 2.66 (m, 1H), 2.33 (m, 4H), 2.12 (t, J=10.8 Hz, 1H), 1.07 (d, J=6.4 Hz, 3H), 0.85 (d, J=6.4 Hz, 3H); MS m/z: 221 (M+1).

5.2.m N⁵-Methyl-N⁵-(1-methylpyrrolidin-3-yl)pyridin-2,5-yldiaminze

¹H NMR (400 MHz, CDCl₃) δ 7.78 (d, J=2.8 Hz, 1H), 7.16 (dd, J₁=8.8 Hz, J₂=2.8 Hz, 1H), 6.47 (d, J=8.8 Hz, 1H), 4.12 (br s, 2H), 3.97-4.04 (m, 1H), 2.72 (s, 3H), 2.60-2.70 (m, 2H), 2.50-2.56 (m, 2H), 2.34 (s, 3H), 2.04-2.10 (m, 1H), 1.77-1.83 (m, 1H); MS m/z: 207 (M+1).

5.2.n 5-3-Methylpiperazin-1-yl)pyridin-2-ylamine

¹H NMR (400 MHz, CDCl₃) δ 7.74 (d, J=2.8 Hz, 1H), 7.15 (dd, J₁=8.8 Hz, J₂=2.8 Hz, 1H), 6.48 (d, J=8.8 Hz, 1H), 4.33 (m, 1H), 4.21 (br s, 2H), 3.92-3.96 (m, 1H), 3.19-3.26 (m, 2H), 3.08-3.11 (m, 1H), 2.82 (dd, J₁=11.6 Hz, J₂=4.0 Hz, 1H), 2.61-2.68 (m, 1H), 1.48 (s, 9H), 1.32 (d, J=6.8 Hz, 3H); MS m/z: 293 (M+1).

5.2.o 5-(3,5-Dimethylpiperazin-1-yl)pyridin-2-ylamine

¹H NMR (400 MHz, CDCl₃) δ 7.76 (d, J=2.8 Hz, 1H), 7.16 (dd, J₁=8.8 Hz, J₂=2.8 Hz, 1H), 6.50 (d, J=8.8 Hz, 1H), 4.18-4.24 (m, 2H), 3.08-3.11 (m, 2H), 2.80 (dd, J₁=11.6 Hz, J₂=4.0 Hz, 1H), 1.49 (s, 9H), 1.37 (d, J=6.8 Hz, 6H); MS m/z: 307 (M+1).

5.2.p N⁵-(2-Methoxyethyl)-N⁵-methylpyridin-2,5-yldiamine

MS m/z: 182 (M+1).

5.2.q 5-(4-Methoxypiperidin-1-yl)pyridin-2-ylamine

MS m/z: 208 (M+1).

5.2.r 5-(1-Methylpiperidin-3-yl)pyridin-2-amine

¹H NMR (300 MHz, CDCl₃) δ 7.93 (d, J=2.2 Hz, 1H), 7.31 (dd, J=2.4 Hz, 8.4 Hz, 1H), 6.45 (d, J=8.4 Hz, 1H), 4.37 (br s, 2H), 2.83-2.92 (m, 2H), 2.64-2.77 (m, 1H), 2.29 (s, 3H), 1.65-1.94 (m, 5H), 1.20-1.45 (m, 1H); MS m/z: 192 (M+1).

5.2.s Ethyl 3-(6-aminopyridin-3-yl)propanoate

¹H NMR (400 MHz, CDCl₃) δ 7.92 (d, J=2.4 Hz, 1H), 7.29 (dd, J=8.0 Hz, 2.4 Hz, 1H), 6.45 (d, J=8.0 Hz, 1H), 4.31 (brs, 2H), 4.12 (q, J=7.2 Hz, 2H), 2.81 (t, J=7.6 Hz, 2H), 2.55 (t, J=7.6 Hz, 2H), 1.24 (t, J=7.2 Hz, 3H); MS m/z: 195 (M+1).

5.2.t 4-(6-aminopyridin-3-yl)-1-methylpiperazin-2-one

¹H NMR (400 MHz, CDCl₃) δ 7.76-7.77 (m, 1H), 7.12-7.17 (m, 1H), 6.49-6.53 (m, 1H), 3.71 (s, 2H), 3.42-3.80 (m, 2H), 3.27-3.38 (m, 2H), 3.02 (s, 3H); MS m/z: 207 (M+1).

5.2.u 1-(6-aminopyridin-3-yl)-4-benzylpiperazin-2-one

¹H NMR (400 MHz, CDCl₃) δ 7.98-7.99 (m, 1H), 7.26-7.40 (m, 1H), 6.47-6.51 (m, 1H), 4.30-4.70 (br, s2H), 3.59-3.64 (m, 4H), 3.32 (2H, s), 2.75-2.83 (m, 2H).

### Example 6

### Preparation of 8

6.1 Ullmann Cross-Coupling

To a solution of 24.6 mmol of 9 and 27.3 mmol of 7 in 50 mL of 1,4-dioxane was added 4.92 mmol of copper (I) iodide followed by the addition of 49.2 mmol of K₃PO₄ and 4.92 mmol of trans-cyclohexanediamine, then the resulting mixture was stirred at 100° C. for 12 h. The reaction mixture was cooled to room temperature and concentrated in vacuo. The residue was diluted with CHCl₃, poured into water, and insoluble material was removed by celite filtration. The filtrate was extracted with CHCl₃, dried over MgSO₄ and concentrated in vacuo. The crude product was purified by column chromatography to give 7.87 mmol of nitro derivative.

A solution of 7.66 mmol of nitro derivative and 0.5 g of Pd/C (10%) in 150 mL of methanol was stirred overnight under H₂ (1 atm). After filtering through celite, the solution was concentrated under reduced pressure to give 4.75 mmol of 8.

6.2 Results

Analytical data for an exemplary compound of structure 8 are provided below.

6.2.a 4-(6-Aminopyridin-3-yl)-1-benzyl-1,4-diazepan-5-one

¹H NMR (400 MHz, DMSO-d₆) δ 7.70 (d, J=2.4 Hz, 1H), 7.17 (dd, J₁=8.8 Hz, J₂=2.4 Hz, 1H), 7.30-7.36 (m, 5H), 6.40 (d, J=8.8 Hz, 1H), 5.90 (br s, 2H), 3.66-3.72 (m, 2H), 3.59 (br s, 2H), 2.59-2.71 (m, 6H); MS m/z: 327 (M+1).

### Example 7

### Preparation of 12

7.1 Halogenation

To a solution of 30.7 mmol of 2 and 5 mL of bromine in 48 mL of hydrobromic acid (48%) at 0° C. was added 24 mL (25 M) of aqueous NaNO₂. The mixture was stirred for 1 h at rt before it was neutralized by 145 mL of 3M NaOH. The aqueous solution was extracted with ethyl acetate, and the organic phase was washed with saturated NaCl, dried over MgSO₄, and concentrated under a reduced pressure. The crude product was purified by column chromatography to give 24.6 mmol of 12.

7.2 Results

Analytical data for exemplary compounds of structure 12 are provided below.

7.2.a 2-Bromo-5-chloro-pyridine

¹H NMR (300 MHz, DMSO-d₆) δ 8.47 (d, J=2.8 Hz, 1H), 7.89 (dd, J=8.5 Hz, J₂=2.7 Hz, 1H), 7.69 (d, J=8.5 Hz, 1H); MS m/z: 192 (M+1).

7.2.b 2-Bromo-5-(4-fluoro-phenyl)-pyridine

¹H NMR (300 MHz, DMSO-d₆) δ 8.68 (d, J=2.4 Hz, 1H), 8.03 (dd, J₁=8.3 Hz, J₂=2.6 Hz, 1H), 7.80-7.70 (m, 3H), 7.34 (d, J=6.6 Hz, 1H), 7.32 (d, J=6.8 Hz, 1H); MS m/z: 252 (M+1).

### Example 8

### Preparation of 14

8.1 Stannylation

To a solution of 17.4 mmol of 13 in 60 mL of dry THF at −78° C. under N₂ was added 19.2 mmol of n-BuLi (2.5 M in hexane), and the resulting brown solution was stirred for 30 min before 20.9 mmol of Bu₃SnCl was added. The reaction mixture was allowed to warm to room temperature overnight. After the reaction was quenched with saturated NH₄Cl and the mixture was extracted with ethyl acetate, the combined organic phase was washed with saturated NaCl, dried over MgSO₄, and concentrated under reduced pressure. The crude product was purified by column chromatography on silica gel to give 10.5 mmol of 14.

8.2 Results

Analytical data for exemplary compounds of structure 14 are provided below.

8.2.a 4-Methyl-2-tributylstannanyl-pyridine

¹H NMR (300 MHz, CDCl₃) δ 8.57 (d, J=5.0 Hz, 1H), 7.21 (s, 1H), 6.93 (d, J=4.7 Hz, 1H), 2.29 (s, 3H), 1.61-1.47 (m, 6H), 1.39-1.29 (m, 6H), 1.16-1.08 (m, 6H), 0.87 (t, J=7.3 Hz, 9H); MS m/z: 384 (M+1).

8.2.b 2-Methoxy-6-tributylstannanyl-pyridine

¹H NMR (300 MHz, CDCl₃) δ 7.39 (dd, J₁=8.3 Hz, J₂=6.9 Hz, 1H), 6.98 (d, J=6.1 Hz, 1H), 6.55 (d, J=8.4 Hz, 1H), 3.93 (s, 3H), 1.62-1.53 (m, 6H), 1.38-1.27 (m, 6H), 1.12-1.05 (m, 6H), 0.89 (t, J=5.9 Hz, 9H); MS m/z: 400 (M+1).

8.2.c 5-Methyl-2-tributylstannanyl-pyridine

¹H NMR (300 MHz, CDCl₃) δ 8.56 (s, 1H), 7.30-7.24 (m, 2H), 2.25 (s, 3H), 1.58-1.44 (m, 6H), 1.36-1.25 (m, 6H), 1.11-1.04 (m, 6H), 0.86 (t, J=7.1 Hz, 9H); MS m/z: 384 (M+1).

8.2.d 4-Pyrrolidin-1-yl-2-tributylstannanyl-pyridine

¹H NMR (300 MHz, DMSO-d₆) δ 8.14 (d, J=4.5 Hz, 1H), 6.68-6.64 (m, 1H), 6.59 (d, J=2.4 Hz, 1H), 3.41 to 3.39 (m, 4H), 1.97 (bs, 4H), 1.58-1.41 (m, 6H), 1.38-1.22 (m, 6H), 1.20-1.00 (m, 6H), 0.83 (t, J=7.3 Hz, 9H); MS m/z: 439 (M+1).

### Example 9

### Preparation of 16

9.1 Stille Cross-Coupling

A mixture of 30 mmol of 15, 30 mmol of 14, and 1.5 mmol of Pd(PPh₃)₄ in 250 mL of dry toluene was stirred at 70° C. for 2 days under N₂. The reaction was quenched with 100 mL of saturated NH₄Cl. After the mixture was extracted with EtOAc, the organic phase was washed with saturated NaCl, dried over MgSO₄, and concentrated under reduced pressure. The residue was purified by column chromatography to give 17.6 mmol of 16.

9.2 Results

Analytical data for exemplary compounds of structure 16 are provided below.

9.2.a 6-Bromo-[2,2′]bipyridinyl

¹H NMR (300 MHz, DMSO-d₆) δ 8.68 (d, J=4.7 Hz, 1H), 8.37 (d, J=7.6 Hz, 1H), 8.25 (d, J=8.0 Hz, 1H), 7.95 (dt, J₁=7.8 Hz, J₂=1.7 Hz, 1H), 7.89 (t, J=7.9 Hz, 1H), 7.69 (d, J=7.9 Hz, 1H), 7.50-7.46 (m, 1H); MS m/z: 235 (M+1).

9.2.b 2-Bromo-6-thiazol-2-yl-pyridine

¹H NMR (300 MHz, DMSO-d₆) δ 8.10 (d, J=7.7 Hz, 1H), 7.99 (d, J=3.1 Hz, 1H), 7.91 (d, J=3.1 Hz, 1H), 7.87 (t, J=7.8 Hz, 1H), 7.69 (d, J=7.8 Hz, 1H); MS m/z: 241 (M+1).

9.2.c 2-(6-Bromo-pyridin-2-yl)-pyrazine

¹H NMR (300 MHz, DMSO-d₆) δ 9.39 (d, J=1.2 Hz, 1H), 8.75 (s, 2H), 8.32 (d, J=7.7 Hz, 1H), 7.94 (t, J=7.8 Hz, 1H), 7.77 (dd, J₁=8.0 Hz, J₂=0.7 Hz, 1H); MS 7 m/z: 236 (M+1).

9.2.d 6-Chloro-3,5-bis(trifluoromethyl)-2,2′-bipyridine

¹H NMR (400 MHz, CDCl₃) δ 8.72-8.74 (m, 1H), 8.41 (s, 1H), 7.79-7.89 (m, 2H), 7.42-7.45 (m, 1H); MS m/z: 327, 329 (M+1).

9.2.e. 6-Bromo-5-methoxy-2,2′-bipyridine

¹H NMR (400 MHz, DMSO-d₆) δ 8.65 (brd, J=4.4 Hz, 1H), 8.36 (d, J=8.0 Hz, 1H), 8.18 (d, J=8.0 Hz, 1H), 7.92 (td, J=8.0 Hz, 1.4 Hz, 1H), 7.66 (d, J=8.8 Hz, 1H), 7.42 (ddd, J=8.0 Hz, 4.4 Hz, 1.4 Hz, 1H), 3.94 (s, 3H); MS m/z: 267, 269 (M+1).

9.2.f. 2-(6-Bromo-5-methoxypyridin-2-yl)pyrazine

¹H NMR (400 MHz, DMSO-d₆) δ 8.34 (d, J=1.2 Hz, 1H), 8.71-8.74 (m, 1H), 8.67 (d, J=2.4 Hz, 1H), 8.33 (d, J=8.8 Hz, 1H), 7.71 (d, J=8.8 Hz, 1H), 3.98 (s, 3H); MS m/z: 268, 270 (M+1).

### Example 10

### Preparation of 16

10.1 Negishi Cross-Coupling

A mixture of 528 mmol of zinc dust and 47.5 mmol of 1,2-dibromoethane was heated with a heat gun until the evolution of ethylene gas was done twice. To a suspension 21.1 mmol of trimethylsilyl chloride and 176 mmol of 12 in 70.0 mL of THF were added. After 30 min 211 mmol of 15 and 2.28 mmol of Pd(PPh₃)₄ in 350 mL of THF were added and the mixture was stirred for 17 h at reflux. The reaction was quenched with saturated NaCl, and insoluble material was removed by celite filtration. The filtrate was extracted with toluene, wased with saturated NaCl, dried over MgSO₄ and concentrated in vacuo. The crude product was purified by column chromatography to give 105 mmol of 16.

10.2 Results

Analytical data for exemplary compounds of structure 16 are provided below.

10.2.a 2-Bromo-3-methoxy-6-(1,3-thiazol-2-yl)pyridine

¹H NMR (400 MHz, DMSO-d₆) δ 8.12 (d, J=7.8 Hz, 1H), 7.94 (d, J=3.2 Hz, 1H), 7.83 (d, J=3.2 Hz, 1H), 7.67 (d, J=7.8 Hz, 1H), 3.97 (s, 3H); MS m/z: 275 (M+1).

10.2.b 2-Bromo-6-(5-methyl-1,3-thiazol-2-yl)pyridine

¹H NMR (400 MHz, DMSO-d₆) δ 8.08 (dd, J₁=7.8 Hz, J₂=0.8 Hz, 1H), 7.88 (t, J=7.8 Hz, 1H), 7.69-7.74 (m, 2H), 2.51 (d, J=2.0 Hz, 3H); MS m/z: 259 (M+1).

10.2.c 2-Bromo-6-(5-ethyl-1,3-thiazol-2-yl)pyridine

¹H NMR (400 MHz, DMSO-d₆) δ 8.08 (dd, J₁=8.0 Hz, J₂=0.8 Hz, 1H), 7.89 (t, J=8.0 Hz, 1H), 7.75 (t, J=0.8 Hz, 1H), 7.70 (dd, J₁=8.0 Hz, J₂=0.8 Hz, 1H), 2.91 (qd, J=J₁=7.6 Hz, J₂=0.8 Hz, 2H), 1.30 (t, J=7.6 Hz, 3H); MS m/z: 272 (M+1).

10.2.d 2-Bromo-6-(5-isopropyl-1,3-thiazol-2-yl)pyridine

¹H NMR (400 MHz, DMSO-d₆) δ 8.09 (dd, J₁=7.8 Hz, J₂=0.8 Hz, 1H), 7.89 (t, J=7.8 Hz, 1H), 7.76 (d, J=0.8 Hz, 1H), 7.71 (dd, J₁=7.8 Hz, J₂=0.8 Hz, 1H), 3.28 (sept, J=6.8 Hz, 1H), 1.34 (d, J=6.8 Hz, 6H); MS m/z: 284 (M+1).

10.2.e 2-Bromo-6-(5-chloro-1,3-thiazol-2-yl)pyridine

¹H NMR (400 MHz, DMSO-d₆) δ 8.10 (d, J=7.6 Hz, 1H), 8.07 (s, 1H), 7.94 (t, J=7.6 Hz, 1H), 7.78 (d, J=7.6 Hz, 1H); MS m/z: 278 (M+1).

10.2f 2-Bromo-6-(5-chloro-1,3-thiazol-2-yl)-3-methoxypyridine

¹H NMR (400 MHz, DMSO-d₆) δ 8.07 (d, J=8.6 Hz, 1H), 7.97 (s, 1H), 7.69 (d, J=8.6 Hz, 1H), 3.97 (s, 3H); MS m/z: 308 (M+1).

### Example 11

### Preparation of 17

11.1 Buchwald Cross-Coupling

A mixture of 40 mmol of 15, 40 mmol of 2 or 8, 0.8 mmol of Pd₂(dba)₃, 1.6 mmol of dppp, and 60 mmol of NaOtBu in 360 mL of dry toluene was stirred at 80° C. overnight under N₂. The reaction was quenched with 100 mL of water and the mixture was diluted with 300 mL of ethyl acetate. After separating the two phases, the organic phase was washed with saturated NaCl, dried over MgSO₄, and concentrated under a reduced pressure. The crude product was purified by column chromatography on silica gel to give 30.7 mmol of 17.

11.2 Results

Analytical data for exemplary compounds of structure 17 are provided below.

11.2.a (6-Bromo-pyridin-2-yl)-(5-chloro-pyridin-2-yl)-amine

¹H NMR (300 MHz, DMSO-d₆) δ 9.64 (s, 1H), 8.22 (d, J=2.4 Hz, 1H), 7.85 (d, J=8.9 Hz, 1H), 7.75 (d, J=2.6 Hz, 1H), 7.05 (d, J=2.8 Hz, 1H), 7.52 (t, J=8.0 Hz, 1H), 7.07 (d, J=8.0 Hz, 1H); MS m/z: 284 (M+1).

### Example 12

### Preparation of 17

12.1 Nucleophilic Replacement

To a solution of 25.9 mmol of 15 in 50 mL of anhydrous THF was added 38.9 mmol of NaH (60% in mineral oil) followed by the addition of 25.9 mmol of 2 or 8, and the resulting mixture was stirred at 50° C. for 8 h. After the reaction was quenched with methanol, the solvent was removed. The residue was dissolved in 100 mL of ethyl acetate and the organic solution was washed with saturated NaCl, dried over MgSO₄, and concentrated under a reduced pressure. The crude product was purified by column chromatography on silica gel to give 16.3 mmol of 17.

12.2 Results

Analytical data for exemplary compounds of structure 17 are provided below.

12.2.a 6-Chloro-3-nitro-N-pyridin-2-ylpyridin-2-amine

¹H NMR (300 MHz, DMSO-d₆) δ 10.64 (br s, 1H), 8.52 (d, J=8.4 Hz, 1H), 8.37-8.42 (m, 2H), 7.78 (t, J=8.4 Hz, 1H), 7.07-7.11 (m, 1H), 6.92 (d, J=8.4 Hz, 1H); MS m/z: 253 (M+1).

12.2.b. Methyl 6-chloro-2-(pyridin-2-ylamino)nicotinate

¹H NMR (400 MHz, CDCl₃) δ 10.15 (br s, 1H), 8.53 (d, J=7.2 Hz, 1H), 8.34-8.38 (m, 2H), 7.74 (t, J=7.2 Hz, 1H), 7.13 (d, J=8.0 Hz, 1H), 7.08 (t, J=7.2 Hz, 1H), 4.23 (s, 3H); MS m/z: 264 (M+1)

12.2.c. 6-Chloro-N,N-dimethyl-2-(pyridin-2-ylamino)nicotinamide

¹H NMR (400 MHz, CDCl₃) δ 8.70 (br s, 1H), 8.33 (d, J=8.4 Hz, 1H), 8.24-8.26 (m, 1H), 7.68 (t, J=8.4 Hz, 1H), 7.46 (d, J=7.6 Hz, 1H), 6.91-6.94 (m, 1H), 6.84 (d, J=7.6 Hz, 1H), 3.08 (6H, s); MS m/z: 277 (M+1).

### Example 13

### Preparation of 18

13.1 Nucleophilic Replacement

To a solution of 10 mmol of 2 or 8 in 100 mL of anhydrous THF was added 30 mmol of NaH (60% in mineral oil) followed by the addition of 12.5 mmol of 16, and the resulting mixture was stirred at 100° C. overnight under N₂. After the reaction was quenched with methanol, the solvents were removed. The residue was dissolved in 100 mL of ethyl acetate and the organic solution was washed with saturated NaCl, dried over MgSO₄, and concentrated under a reduced pressure. The crude product was purified by column chromatography on silica gel to give 4.5 mmol of 18.

Most of 18 was converted to an HCl salt by adding excess 4 M of HCl in 1,4-dioxane to a solution of 18 in MeOH. The pure salts were obtained by removing the solvents under reduced pressure or crystallizing in ethyl acetate.

13.2 Results

Analytical data for exemplary compounds of structure 18 are provided below.

13.2.a [5-(3-Fluoro-phenyl)-pyridin-2-yl]-(6-thiazol-2-yl-pyridin-2-yl)-amine.2HCl

¹H NMR (300 MHz, DMSO-d₆) δ 12.17 (s, 1H), 8.84 (d, J=2.1 Hz, 1H), 8.55 (dd, J=9.0 Hz, J₂=2.3 Hz, 1H), 8.05-7.97 (m, 4H), 7.83 (d, J=7.5 Hz, 1H), 7.71-7.63 (m, 2H), 7.57 (d, J=6.6 Hz, 1H), 7.52 (d, J=7.5 Hz, 1H), 7.25 (dt, J₁=8.5 Hz, J₂=2.3 Hz, 1H); MS m/z: 349 (M+1).

13.2.b [3,3′]Bipyridinyl-6-yl-(6-thiazol-2-yl-pyridin-2-yl)-amine.2HCl

¹H NMR (300 MHz, DMSO-d₆) δ 10.68 (s, 1H), 9.32 (d, J=2.1 Hz, 1H), 8.93-8.84 (m, 3H), 8.39 (dd, J₁=9.1 Hz, J₂=2.6 Hz, 1H), 8.21 (d, J=8.9 Hz, 1H), 8.10 (dd, J₁=8.2 Hz, J₂=5.8 Hz, 1H), 8.00 (d, J=3.3 Hz, 1H), 7.91 (d, J=3.1 Hz, 1H), 7.88 (d, J=7.9 Hz, 1H), 7.71 (d, J=7.1 Hz, 1H), 7.63 (d, J=8.2 Hz, 1H); MS m/z: 332 (M+1).

13.2.c (5-Phenyl-2H-pyrazol-3-yl)-(6-thiazol-2-yl-pyridin-2-yl)-amine.2HCl

¹H NMR (300 MHz, DMSO-d₆) δ 10.50 (s, 1H), 7.99 (d, J=3.0 Hz, 1H), 7.91 (d, J=3.0 Hz, 1H), 7.82-7.68 (m, 3H), 7.57 (d, J=7.3 Hz, 1H), 7.51-7.46 (m, 2H), 7.39 (d, J=7.1 Hz, 1H), 7.21 (d, J=8.4 Hz, 1H), 7.07 (s, 1H); MS m/z: 320 (M+1).

13.2.d 1-[6-(6-Thiazol-2-yl-pyridin-2-ylamino)-pyridin-3-yl]-pyrrolidin-2-one.2HCl

¹H NMR (300 MHz, DMSO-d₆) δ 11.92 (s, 1H), 8.73 (d, J=2.5 Hz, 1H), 8.40 (dd, J₁=9.5 Hz, J₂=2.6 Hz, 1H), 8.03 (d, J=3.2 Hz, 1H), 8.01-7.92 (m, 3H), 7.78 (d, J=7.3 Hz, 1H), 7.43 (d, J=8.2 Hz, 1H), 3.87 (t, J=7.1 Hz, 2H), 2.49 (t, J=9.1 Hz, 2), 2.15-2.05 (m, 2H); MS m/z: 338 (M+1).

13.2.e [5-(4-Methyl-piperazin-1-yl)-pyridin-2-yl]-(6-pyrazin-2-yl-pyridin-2-yl)-amine.2HCl

¹H NMR (300 MHz, DMSO-d₆) δ 11.19 (s, 1H), 9.58 (s, 1H), 8.92 (s, 1H), 8.83 (d, J=2.4 Hz, 1H), 8.17-8.10 (m, 3H), 8.01 (d, J=9.2 Hz, 1H), 7.59 (d, J=9.4 Hz, 1H), 7.49 (d, J=7.8 Hz, 1H), 3.88 (d, J=11.2 Hz, 2H), 3.52 (d, J=11.8 Hz, 2H), 3.26-3.16 (m, 4H), 2.80 (d, J=4.4 Hz, 3H); MS m/z: 348 (M+1).

13.2.f [2,2′] Bipyridinyl-6-yl-[5-(4-fluorophenyl)-4-methyl-pyridin-2-yl]-amine

¹H NMR (300 MHz, DMSO-d₆) δ 9.82 (s, 1H), 8.67 (d, J=3.8 Hz, 1H), 8.34 (d, J=7.9 Hz, 1H), 8.05 (s, 1H), 7.97 (dt, J₁=7.7 Hz, J₂=1.7 Hz, 1H), 7.92 (s, 1H), 7.87 (d, J=7.3 Hz, 1H), 7.79 (t, J=8.2 Hz, 1H), 7.68 (d, J=7.5 Hz, 1H), 7.46-7.41 (m, 3H), 7.29 (d, J=8.8 Hz, 1H), 7.26 (d, J=8.9 Hz, 1H), 2.28 (s, 3H); MS m/z: 357 (+1).

13.2.g (5-Isopropyl-pyridin-2-yl)-(6-pyrazin-2-yl-pyridin-2-yl)-amine.2HCl

¹H NMR (300 MHz, DMSO-d₆) δ 12.79 (s, 1H), 9.57 (d, J=1.1 Hz, 1H), 8.87 (d, J=1.2 Hz, 1H), 8.81 (d, J=2.4 Hz, 1H), 8.39 (d, J=2.4 Hz, 1H), 8.24 (dd, J₁=9.1 Hz, J₂=2.1 Hz, 1H), 8.18-8.12 (m, 2H), 7.66 (d, J=9.0 Hz, 1H), 7.50 (dd, J₁=6.3 Hz, J₂=2.8 Hz, 1H), 3.07-2.98 (m, 1H), 1.25 (d, J=7.0 Hz, 6H); MS m/z: 292 (M+1).

13.2.h [5-(5-Methyl-furan-2-yl)-pyridin-2-yl]-(6-thiazol-2-yl-pyridin-2-1-amine.2HCl

¹H NMR (300 MHz, DMSO-d₆) δ 10.99 (s, 1H), 8.59 (d, J=1.9 Hz, 1H), 8.16 (d, J=7.0 Hz, 1H), 8.01 (d, J=2.6 Hz, 1H), 8.00 (d, J=2.9 Hz, 1H), 7.95-7.84 (m, 2H), 7.72 (d, J=7.5 Hz, 1H), 7.50 (d, J=8.3 Hz, 1H), 6.88 (d, J=3.1 Hz, 1H), 6.22 (d, J=2.6 Hz, 1H), 2.34 (s, 3H); MS m/z: 335 (M+1).

13.2.i (5-Morpholin-4-yl-pyridin-2-yl-(6-pyrazin-2-yl-pyridin-2-yl)-amine.2HCl

¹H NMR (300 MHz, DMSO-d₆) δ 12.36 (s, 1H), 9.57 (s, 1H), 8.97 (d, J=1.4 Hz, 1H), 8.83 (d, J=2.4 Hz, 1H), 8.17-8.01 (m, 3H), 8.12 (s, 1H), 7.54 (d, J=9.2 Hz, 1H), 7.44 (dd, J₁=7.5 Hz, J₂=1.6 Hz, 1H), 3.74 (dd, J₁=9.2 Hz, J₂=4.2 Hz, 4H); 3.18 (dd, J₁=9.2 Hz, J₂=4.7 Hz, 4H); MS m/z: 335 (M+1).

13.2.j [3,5-Bis(trifluoromethyl)-2,2′-bipyridin-6-yl](pyridin-2-yl)amine

¹H NMR (400 MHz, CDCl₃) δ 8.74 (d, J=4.8 Hz, 1H), 8.31 (d, J=4.8 Hz, 1H), 8.33 (d, J=8.0 Hz, 1H), 8.23 (s, 1H), 7.87 (t, J=7.6 Hz, 1H), 7.86 (t, J=7.6 Hz, 1H), 7.71 (d, J=8.0 Hz, 1H), 7.65 (t, J=7.6 Hz, 1H), 7.44 (dd, J=4.8, 7.6 Hz, 1H), 7.00 (t, J=7.6 Hz, 1H); MS m/z: 385 (M+1).

### Example 14

### Preparation of 18

14.1 Buchwald Cross-Coupling

A mixture of 1.1 mmol of 16, 1.2 mmol of 2 or 8, 0.045 mmol of Pd₂(dba)₃, 0.09 mmol of dppp, and 1.58 mmol of NaOtBu in 10 mL of dry toluene was stirred at 70° C. overnight under N₂. The reaction was quenched with water and the mixture was diluted with 150 mL of ethyl acetate. After separating the two phases, the organic phase was washed with saturated NaCl, dried over MgSO₄, and concentrated under a reduced pressure. The crude product was purified by column chromatography on silica gel to give 0.97 mmol of 18.

Most of 18 were converted to HCl salt by adding excess 4 M of HCl in 1,4-dioxane to a solution of 18 in MeOH. The pure salts were obtained by removing the solvents under reduced pressure or crystallizing in ethyl acetate.

14.2 Results

Analytical data for exemplary compounds of structure 18 are provided below.

14.2.a [2,2′]Bipyridinyl-6-yl-pyridin-2-yl-amine.3HCl

¹H NMR (300 MHz, DMSO-d₆) δ 12.83 (s, 1H), 8.92 (d, J=4.7 Hz, 1H), 8.56 (d, J=5.4 Hz, 1H), 8.49 (d, J=7.9 Hz, 1H), 8.28-8.15 (m, 4H), 7.56 (t, J=5.8 Hz, 1H), 7.59 (d, J=8.5 Hz, 2H), 7.34 (t, J=7.0 Hz, 1H); MS m/z: 249 (M+1).

14.2.b [2,2′]Bipyridinyl-6-yl-(5-fluoro-pyridin-2-yl)-amine.3HCl

¹H NMR (300 MHz, DMSO-d₆) δ 13.00 (s, 1H), 8.96 (d, J=4.1 Hz, 1H), 8.66 (d, J=2.9 Hz, 1H), 8.45 (d, J=8.0 Hz, 1H), 8.31 (t, J=8.0 Hz, 1H), 8.15 (d, J=7.5 Hz, 1H), 8.13 (d, J=7.6 Hz, 1H), 7.98 (dt, J=8.7 Hz, J₂=3.0 Hz, 1H), 7.71-7.64 (m, 2H), 7.58 (d, J=9.2 Hz, 1H); MS m/z: 267 (M+1).

14.2.c [2,2′]Bipyridinyl-6-yl-(5-chloro-pyridin-2-yl)-amine.3HCl

¹H NMR (300 MHz, DMSO-d₆) δ 12.80 (s, 1H), 8.87 (d, J=4.4 Hz, 1H), 8.42 (d, J=6.3 Hz, 2H), 8.22-8.12 (m, 3H), 7.69 (dd, J₁=7.2 Hz, J₂=6.2 Hz, 1H), 7.56 (d, J=7.8 Hz, 1H), 7.42 (s, 1H), 7.19 (d, J=6.1 Hz, 1H); MS m/z: 283 (M+1).

14.2.d [2,2′]Bipyridinyl-6-yl-(3,5-dichloro-pyridin-2-yl)-anine

¹H NMR (300 MHz, DMSO-d₆) δ 8.68 (s, 1H), 8.65 (d, J=4.2 Hz, 1H), 8.35-8.30 (m, 2H), 8.18 (d, J=2.3 Hz, 1H), 8.0-7.86 (m, 4H), 7.42 (dd, J₁=6.2 Hz, J₂=4.7 Hz, 1H);

MS m/z: 317 (M+1).

14.2.e [2,2′]Bipyridinyl-6-yl-[5-(4-fluoro-phenyl)-pyridin-2-yl]-amine

¹H NMR (300 MHz, DMSO-d₆) δ 9.94 (s, 1H), 8.67 (d, J=3.8 Hz, 1H), 8.56 (s, 1H), 8.35 (d, J=8.0 Hz, 1H), 8.04 (s, 1H), 8.04-8.00 (m, 1H), 7.96 (dt, J₁=7.7 Hz, J₂=1.8 Hz, 1H), 7.89 (d, J=7.6 Hz, 1H), 7.81 (t, J=7.5 Hz, 1H), 7.76-7.69 (m, 3H), 7.45-7.41 (m, 1H), 7.28 (d, J=8.9 Hz, 2H); MS m/z: 343 (M+1).

14.2.f [2,2′]Bipyridinyl-6-yl-(4-methyl-pyridin-2-yl)-amine.2HCl

¹H NMR (300 MHz, DMSO-d₆) δ 12.81 (s, 1H), 8.87 (d, J=4.2 Hz, 1H), 8.43-8.40 (m, 2H), 8.21-8.12 (m, 3H), 8.68 (dd, J₁=7.2 Hz, J₂=5.3 Hz, 1H), 7.55 (d, J=7.9 Hz, 1H), 7.41 (s, 1H), 7.19 (d, J=6.1 Hz, 1H), 2.45 (s, 3H); MS m/z: 263 (M+1).

14.2.g N,N-Dipyridin-2-yl-2,2′-bipyridin-6-amine dihydrochloride

¹H NMR (400 MHz, CDCl₃) δ 8.66 (d, J=4.8 Hz, 1H), 8.50 (d, J=2.4 Hz, 1H), 8.39 (d, J=8.4 Hz, 1H), 8.34-8.36 (m, 3H), 7.79 (t, J=7.6 Hz, 1H), 6.56-7.63 (m, 3H), 7.27 (t, J=4.8 Hz, 1H), 7.07 (d, J=8.4 Hz, 2H), 6.98-7.02 (m, 2H); MS m/z: 326 (M+1).

14.2.h 1-Methyl-4-(6-{[6-(1,3-thiazol-2-yl)pyridin-2-yl]amino}pyridin-3-yl)piperazin-2-one dihydrochloride

¹H NMR (400 MHz, DMSO-d₆) δ 11.96 (br s, 1H), 8.08-8.12 (m, 1H), 8.06 (d, J=2.9 Hz, 1H), 7.96-8.02 (m, 3H), 7.77-7.83 (m, 2H), 7.38 (d, J=8.3 Hz, 1H), 3.87 (s, 2H), 3.56-3.60 (m, 2H), 3.40-3.50 (m, 2H), 2.93 (s, 3H); MS m/z: 367 (M+1).

14.2.i 4-Benzyl-1-(6-{[3-methoxy-6-(1,3-thiazol-2-yl)pyridin-2-yl]amino}pyridin-3-yl)piperazin-2-one dihydrochloride

¹H NMR (400 MHz, DMSO-d₆) δ 8.76-9.04 (m, 1H), 8.48 (d, J=9.2 Hz, 1H), 8.35 (d, J=2.4 Hz, 1H), 7.90-7.98 (m, 2H), 7.80 (d, J=2.9 Hz, 1H), 7.76 (d, J=8.3 Hz, 1H), 7.62-7.72 (m, 2H), 7.46 7.58 (m, 4H), 4.49 (s, 2H), 3.45-4.35 (m, 9H); MS m/z: 473 (M+1).

14.2.j N²-[3-Methoxy-6-(1,3-thiazol-2-yl)pyridin-2-yl]-N⁵-methyl-N⁵-(1-methylpyrrolidin-3-yl)pyridine-2,5-diamine trihydrochloride

¹HNMR (400 MHz, DMSO-d₆) δ11.86 (brs, 0.6H), 11.59 (brs, 0.4H), 10.12 (br s, 1H), 8.41-8.43 (m, 1H), 8.08 (m, 1H), 7.99 (d, J=3.0 Hz, 1H), 7.93 (d, J=3.5 Hz, 1H), 7.78-7.81 (m, 2H), 7.58 (d, J=8.3 Hz, 1H), 4.92 (m, 0.6H), 4.66 (m, 0.4H), 4.01 (s, 3H), 3.03-3.73 (m, 4H), 2.93-2.96 (m, 3H), 2.81-2.85 (m, 3H), 2.17-2.31 (m, 2H); MS m/z: 397 (M+1).

14.2.k 3-Methoxy-6-(1,3-thiazol-2-yl)-N-[5-(2,4,5-trimethylpiperazin-1-yl)pyridin-2-yl]pyridin-2-amine trihydrochloride

¹H NMR (400 MHz, DMSO-d₆) δ 11.49 (br s, 1H), 9.78 (br s, 0.3H), 9.38 (br s, 0.7H), 8.44-8.47 (m, 1H), 7.92-8.15 (m, 3H), 7.79 (d, J=8.3 Hz, 1H), 7.76 (d, J=3.4 Hz, 1H), 7.56 (d, J=8.3 Hz, 1H), 4.13 (m, 0.3H), 4.02 (s, 3H), 3.64 (m, 1H), 3.18-3.50 (m, 4H), 2.92 (m, 0.7H), 2.80 (m, 3H), 1.33-1.35 (m, 3H), 1.23 (m, 0.9H), 0.95 (d, J=5.9 Hz, 2.1H); MS m/z: 411 (M+1).

14.2.l N⁵-1-Azabicyclo[2.2.2]oct-3-yl-N²-[3-methoxy-6-(1,3-thiazol-2-yl)pyridin-2-yl]pyridine-2,5-diamine trihydrochloride

¹H NMR (400 MHz, DMSO-d₆) δ 10.65 (br s, 1H), 10.38 (br s, 1H), 8.12 (d, J=9.3 Hz, 1H), 7.97 (d, J=2.9 Hz, 1H), 7.91 (dd, J₁=9.8 Hz, J₂=2.9 Hz, 1H), 7.86 (d, J=3.4 Hz, 1H), 7.83 (d, J=8.3 Hz, 1H), 7.80 (d, J=2.9 Hz, 1H), 7.63 (d, J=8.3 Hz, 1H), 4.02 (s, 3H), 3.72-3.99 (m, 3H), 3.18-3.32 (m, 3H), 3.01 (m, 1H), 2.22-2.23 (m, 1H), 2.13 (m, 1H), 1.91-1.96 (m, 2H), 1.73 (m, 1H); MS m/z: 409 (M+1).

14.2.m N-{5-[3-(Dimethylamino)pyrrolidin-1-yl]pyridin-2-yl}-3-methoxy-6-(1,3-thiazol-2-yl)pyridin-2-amine dihydrochloride

¹H NMR (400 MHz, DMSO-d₆) δ 10.82 (br s, 1H), 8.26 (d, J=9.3 Hz, 1H), 7.95 (d, J=2.9 Hz, 1H), 7.82 (d, J=2.9 Hz, 1H), 7.78 (d, J=2.5 Hz, 1H), 7.73 (d, J=8.3 Hz, 1H), 7.64 (m, 1H), 7.53 (d, J=8.3 Hz, 1H), 4.01 (s, 3H), 3.17-3.70 (m, 5H), 2.83, 2.84 (each s, 3H×2), 2.50-2.51 (m, 1H), 2.30-2.32 (m, 1H); MS m/z: 397 (M+1).

14.2.n 4-(6-{[3-Methoxy-6-(1,3-thiazol-2-yl)pyridin-2-yl]amino}pyridin-3-yl)-1-methyl-1,4-diazepan-5-one dihydrochloride

¹H NMR (400 MHz, DMSO-d₆) δ 11.30-11.50 (br, 1H), 8.90-9.10 (br, 1H), 8.47 (d, 9.3 Hz, 1H), 8.34 (d, J=2.5 Hz, 1H), 7.96 (dd, J₁=9.3 Hz, J₂=2.5 Hz, 1H), 7.95 (d, J=2.9 Hz, 1H), 7.82 (d, J=2.9 Hz, 1H), 7.77 (d, J=8.8 Hz, 1H), 7.55 (d, J=8.8 Hz, 1H), 4.40-4.56 (m, 1H), 4.01 (s, 3H), 3.35-3.95 (m, 6H), 2.84 (s, 1.5H), 2.83 (s, 1.5H), 2.64-2.76 (m, 1H); MS m/z: 411 (M+1).

14.2.o 1-(6-{[6-(5-Chloro-1,3-thiazol-2-yl)-3-methoxypyridin-2-yl]amino}pyridin-3-yl)-4-methylpiperazin-2-one hydrochloride

¹H NMR (400 MHz, DMSO-d₆) δ 11.50-11.75 (br, 1H), 8.41 (d, J=9.0 Hz, 1H), 8.39 (s, 1H), 8.30 (d, J=2.5 Hz, 1H), 7.92 (dd, J₁=9.0 Hz, J₂=2.5 Hz, 1H), 7.91 (s, 1H), 7.64 (d J=8.3 Hz, 1H), 7.48 (d, J=8.3 Hz, 1H), 4.00-4.04 (m, 2H), 3.99 (s, 3H), 3.56-3.74 (m, 2H), 3.30-3.40 (m, 2H), 2.91 (s, 3H); MS m/z: 431 (M+1).

14.2.p 5-(4-Methyl-1,4-diazepan-1-yl)-N-[6-(1,3-thiazol-2-yl)pyridin-2-yl]pyridin-2-amine trihydrochloride

¹H NMR (400 MHz, DMSO-d₆) δ 11.80-12.00 (br, 1H), 11.10-11.24 (br, 1H), 8.07 (d, J=2.9 Hz, 1H), 7.96-8.05 (m, 3H), 7.89 (d, J=3.0 Hz, 1H), 7.83 (d, J=9.8 Hz, 1H), 7.80 (d, J=7.8 Hz, 1H), 7.31 (d, J=8.3 Hz, 1H), 3.76-3.96 (m, 2H), 3.38-3.60 (m, 4H), 3.10-3.30 (m, 2H), 2.80 (s, 1.5H), 2.79 (s, 1.5H), 2.30-2.48 (m, 1H), 2.12-2.24 (m, 1H); MS m/z: 367 (M+1).

14.2.q N-[6-(5-Ethyl-1,3-thiazol-2-yl)pyridin-2-yl]-5-(4-methylpiperazin-1-yl)pyridin-2-amine trihydrochloride

¹H NMR (400 MHz, DMSO-d₆) δ 11.92 (brs, 1H), 11.36 (brs, 1H), 8.08-8.13 (m, 2H), 7.97 (t, J=7.8 Hz, 1H), 7.87 (d, J=9.3 Hz, 1H), 7.78 (s, 1H), 7.75 (d, J=7.3 Hz, 1H), 7.36 (d, J=8.3 Hz, 1H), 3.88 (br d, J=11.3 Hz, 4H), 3.52 (br d, J=11.3 Hz, 2H), 3.18-3.29 (m, 4H), 2.95 (q, J=7.3 Hz, 2H), 2.81 (d, J=4.4 Hz, 3H), 1.34 (t, J=7.3 Hz, 3H); MS m/z: 381 (M+1).

14.2.r 1-(6-{[6-(5-Ethyl-1,3-thiazol-2-yl)pyridin-2-yl]amino}pyridin-3-yl)piperidin-2-one monohydrochloride

¹H NMR (400 MHz, DMSO-d₆) δ 11.12 (br s, 1H), 8.47 (s, 1H), 7.91 (t, J=7.3 Hz, 1H), 7.75 (s, 1H), 7.69 (d, J=7.3 Hz, 1H), 7.6 (s, 2H), 7.47 (d, J=8.3 Hz, 1H), 3.67 (t, J=5.8 Hz, 2H), 2.93 (q, J=7.3 Hz, 2H), 2.43 (t, J=5.8 Hz, 2H), 1.82-1.94 (m, 4H), 1.32 (t, J=7.3 Hz, 3H); MS m/z: 380 (M+1).

14.2.s N-[6-(5-Ethyl-1,3-thiazol-2-yl)pyridin-2-yl]-5-pyrrolidin-1-ylpyridin-2-amine dihydrochloride

¹H NMR (400 MHz, DMSO-d₆) δ 11.48 (br s, 1H), 7.95 (t, J=7.8 Hz, 1H), 7.78 (s, 1H), 7.75 (br s, 1H), 7.73 (d J=7.3 Hz, 1H), 7.63-7.68 (m, 2H), 7.20 (d, J=8.3 Hz, 1H), 3.33 (br s, 4H), 2.95 (q, J=7.3 Hz, 2H), 2.02 (br s, 4H), 1.33 (t, J=7.3 Hz, 3H); MS m/z: 352 (M+1).

14.2.t N-[6-(5-Ethyl-1,3-thiazol-2-yl)pyridin-2-yl]-5-piperidin-1-ylpyridin-2-amine dihydrochloride

¹H NMR (400 MHz, DMSO-d₆) δ 11.14 (br s, 1H), 8.45 (br s, 1H), 8.28 (dd, J₁=8.8 Hz, J₂=2.4 Hz, 1H), 8.03 (br s, 1H), 7.90 (t, J=7.8 Hz, 1H), 7.75 (s, 1H), 7.70 (d, J=8.6 Hz, 1H), 7.42 (d, J=8.3 Hz, 1H), 2.92 (q, J=7.3 Hz, 2H), 1.87 (br s, 4H), 1.63 (br s, 2H), 1.33 (t, J=7.3 Hz, 3H); MS m/z: 366 (M+1).

14.2.u N-[6-(5-Methyl-1,3-thiazol-2-yl)pyridin-2-yl]-5-morpholin-4-ylpyridin-2-amine dihydrochloride

¹H NMR (400 MHz, DMSO-d₆) δ 10.81 (brs, 1H), 10.43 (brs, 1H), 8.81-9.45 (m, 2H), 7.00-7.76 (m, 5H), 3.60-5.00 (m, 1H); MS m/z: 354 (M+1).

14.2.v 1-(6-{[6-(5-Chloro-1,3-thiazol-2-yl)-3-methoxypyridin-2-yl]amino}pyridin-3-yl)pyrrolidin-2-one monohydrochloride

¹H NMR (400 MHz, DMSO-d₆) δ 9.52 (br s, 1H), 8.77 (d, J=3.0 Hz, 1H), 8.43 (dd, J₁=9.3 Hz, J₂=2.4 Hz, 1H), 8.34 (d, J=9.3 Hz, 1H), 7.94 (s, 1H), 7.72 (d, J=8.3 Hz, 1H), 7.56 (d, J=8.3 Hz, 1H), 4.01 (s, 3H), 3.89 (t, J=6.9 Hz, 2H), 2.54 (t, J=8.3 Hz, 2H), 2.06-2.18 (m, 2H); MS m/z: 402 (M+1).

14.2.w 5-(4-Isopropylpiperazin-1-yl)-N-[6-(1,3-thiazol-2-yl)pyridin-2-yl]pyridin-2-amine t7-dihydrochloride

¹HNMR (400 MHz, DMSO-d₆) δ 11.07(brs, 1H), 11.61 (brs, 1H), 8.03-8.13 (m, 3H), 7.94-8.01 (m, 2H), 7.40 (d, J=8.3 Hz, 1H), 3.45-4.00 (m, 5H), 3.48-3.40 (m, 2H), 3.20-3.43 (m, 2H), 1.34 (d, J=6.3 Hz, 6H); MS m/z: 381 (M+1).

14.2.x 1-(6-{[6-(5-Methyl-1,3-thiazol-2-yl)pyridin-2-yl]amino}pyridin-3-yl)pyrrolidin-2-one dihydrochloride

¹H NMR (400 MHz, DMSO-d₆) δ 11.84 (br s, 1H), 8.45 (dd, J₁=8.8 Hz, J₂=2.4 Hz, 1H), 7.95-8.00 (m, 3H), 7.74-7.77 (m, 2H), 7.42 (t, J=8.0 Hz, 1H), 3.90 (t, J=6.8 Hz, 2H), 2.56 (s, 3H), 2.53 (t, J=6.8 Hz, 2H), 2.09-2.16 (m, 2H); MS m/z: 352 (M+1).

14.2.y N-[6-(5-Isopropyl-1,3-thiazol-2-yl)pyridin-2-yl]-5-(4-methylpiperazin-1-yl)pyridin-2-amine dihydrochloride

¹H NMR (400 MHz, DMSO-d₆) δ 11.45 (br s, 1H), 8.14 (dd, J₁=9.2 Hz, J₂=2.8 Hz, 1H), 8.07 (d, J=2.8 Hz, 1H), 7.98 (t, J=8.4 Hz, 1H), 7.88 (d, J=9.2 Hz, 1H), 7.76 (d, J=7.2 Hz, H), 7.38 (d, J=8.4 Hz, H), 3.88 (d, J=11.2 Hz, 2H), 3.52 (d, J=11.2 Hz, 2H), 3.15-3.37 (m, 5H), 1.39 (d, J=6.8 Hz, 6H); MS m/z: 395 (M+1).

14.2.z 5-(1-Methylpiperidin-3-yl)-N-(6-pyrazin-2-ylpyridin-2-yl)pyridin-2-amine trihydrochloride

¹HNMR (400 MHz, DMSO-d₆) δ 12.31 (brs, 1H), 10.91 (brs, H), 9.60 (d, J=1.5 Hz, 1H), 8.91 (t, J=2.4 Hz, 1H), 8.84 (d, J=2.4 Hz, 1H), 8.44 (d, J=2.0 Hz, 1H), 8.07-8.22 (m, 3H), 7.74 (d, J=8.8 Hz, H), 7.61 (dd, J₁=6.9 Hz, J₂=2.4 Hz, 1H), 3.40-3.56 (m, 2H), 3.13-3.32 (m, 2H), 2.88-3.00 (m, 1H), 2.77 (s, 1.5H), 2.76 (s, 1.5H), 1.90-2.02 (m, 3H), 1.62-1.74 (m, 1H); MS m/z: 347 (M+1).

Characterization data for some modulators of the present invention are presented in Table 1 below.

TABLE 1
Chemical Name
MS m/z
¹H-NMR δ (ppm)
Solvent

N-(5-Bromopyridin-2-yl)-5-methoxy-2,2′-bipyridin-6-amine ¹H NMR (400 MHz, DMSO-
d₆) δ 8.64 (d, J = 3.6 Hz, 1H) 8.46 (d, J = 8.4 Hz, 1H), 8.26 (s, 1H), 8.37 (s, 1H), 8.22 (d, J = 7.6 Hz,
1H), 8.05 (d, J = 8.4 Hz, 1H), 7.99 (d, J = 8.4 Hz, 1H), 7.92 (t, J = 7.6 Hz, 1H),
7.48 (d, J = 8.4 Hz, 1H), 7.38 (t, J = 5.2 Hz, 1H), 3.97 (s, 3H); MS m/z: 358 (M + 1).
5-Methoxy-N-(5-phenylpyridin-2-yl)-2,2′-bipyridin-6-amine ¹H NMR (400 MHz, DMSO-
d₆) δ 8.62-8.65 (m, 3H), 8.29 (d, J = 7.6 Hz, 1H), 8.19-8.21 (m, 2H), 7.99 (d, J = 8.4 Hz,
1H), 7.95 (t, J = 7.6 Hz, 1H), 7.74 (d, J = 7.6 Hz, 2H), 7.48-7.50 (m, 3H),
7.35-7.39 (m, 2H), 4.00 (s, 3H); MS m/z: 355 (M + 1).
N-[5-(3-Fluorophenyl)pyridin-2-yl]-5-methoxy-2,2′-bipyridin-6-amine ¹H NMR (400 MHz,
DMSO-d₆) δ 8.61-8.67 (m, 3H), 8.23-8.29 (m, 3H), 7.99 (d, J = 8.4 Hz, 1H),
7.95 (t, J = 7.6 Hz, 1H), 7.59-7.63 (m, 2H), 7.49-7.54 (m, 2H), 7.39 (t, J = 5.2 Hz, 1H),
7.19 (t, J = 6.8 Hz, 1H), 4.00 (s, 3H); MS m/z: 373 (M + 1).
N-(5-Nitropyridin-2-yl)-2,2′-bipyridin-6-amine ¹H NMR (400 MHz, DMSO-d₆) δ 10.81 (br
s, 1H), 8.84 (d, J = 2.8 Hz, 1H), 8.68-8.73 (m, 1H), 8.54 (dd, J = 2.8, 9.2 Hz, 1H),
8.37 (d, J = 8.0 Hz, 1H), 8.06 (d, J = 7.6 Hz, 1H), 7.92-8.01 (m, 2H), 7.76 (d, J = 8.4 Hz, 1H),
7.49-7.54 (m, 2H); MS m/z: 294 (M + 1).
5-Methyl-N-[6-(1,3-thiazol-2-yl)pyridin-2-yl]pyridin-2-amine ¹H NMR (400 MHz,
DMSO-d₆) δ 9.78 (s, 1H), 8.11 (s, 1H), 7.97 (d, J = 2.9 Hz, 1H), 7.92 (d, J = 8.8 Hz, 1H),
7.85 (d, J = 2.9 Hz, 1H), 7.78 (t, J = 7.9 Hz, 1H), 7.38-7.61 (m, 3H), 2.25 (s, 3H); MS m/z:
269 (M + 1).
Methyl 6-{[6-(1,3-thiazol-2-yl)pyridin-2-yl]amino}nicotinate ¹H NMR (400 MHz, DMSO-
d₆) δ 10.46 (1H, s), 8.82 (1H, d, J = 2.4 Hz), 8.22 (1H, dd, J = 2.4 Hz, 9.0 Hz), 8.09 (1H, d,
J = 8.8 Hz), 8.00 (1H, d, J = 2.9 Hz), 7.86-7.91 (2H, m), 7.72 (2H, dd, J = 5.9 Hz, 7.8 Hz),
3.85 (3H, s); MS m/z: 313 (M + 1).
5-Hexyl-N-[6-(1,3-thiazol-2-yl)pyridin-2-yl]pyridin-2-amine ¹H NMR (400 MHz, DMSO-
d₆) δ 9.80 (s, 1H), 8.10 (d, J = 2.0 Hz, 1H), 7.99 (d, J = 9.4 Hz, 1H), 7.98 (s, 1H), 7.86 (d, J = 3.4 Hz,
1H), 7.79 (t, J = 8.3 Hz, 1H), 7.61 (d, J = 2.4 Hz, 1H), 7.59 (d, J = 3.4 Hz, 1H),
2.53 (t, J = 7.8 Hz, 2H), 1.52-1.61 (m, 2H), 1.23-1.33 (m6H,), 8.58 (t, J = 6.9 Hz3, H);
MS m/z: 339 (M + 1).
5-tert-Butyl-N-[6-(1,3-thiazol-2-yl)pyridin-2-yl]pyridin-2-amine dihydrochloride ¹H NMR
(400 MHz, DMSO-d₆) δ 12.96 (br d, 1H), 8.52 (dd, J = 2.4, 9.3 Hz, 1H), 8.36 (d, J = 1.9 Hz,
1H), 8.04-8.10 (m, 2H), 8.03 (d, J = 3.5 Hz, 1H), 7.89-7.98 (m, 2H), 7.47 (d, J = 8.2 Hz,
1H), 1.36 (s, 9H),; MS m/z: 311 (M + 1).
5-Ethyl-N-[6-(1,3-thiazol-2-yl)pyridin-2-yl]pyridin-2-amine dihydrochloride ¹H NMR
(400 MHz, DMSO-d₆) δ 10.75 (br s, 1H), 8.25 (s, 1H), 8.03 (d, J = 3.0 Hz, 1H),
7.90-7.94 (m, 3H), 7.83 (d, J = 7.8 Hz, 1H), 7.75 (d, J = 7.8 Hz, 1H), 7.45 (d, J = 8.3 Hz, 1H), 2.68 (q,
J = 7.3 Hz, 2H), 1.23 (t, J = 7.3 Hz, 3H); MS m/z: 283 (M + 1).
5-[2-(Benzyloxy)ethyl]-N-[6-(1,3-thiazol-2-yl)pyridin-2-yl]pyridin-2-amine
dihydrochloride ¹H NMR (400 MHz, DMSO-d₆) δ 12.58 (br s, 1H), 8.39 (s, 1H), 8.24 (d, J = 8.4 Hz,
1H), 8.06-8.08 (m, 2H), 8.01 (d, J = 3.6 Hz, 1H), 7.89 (d, J = 8.4 Hz, 1H),
7.83 (d, J = 8.4 Hz, 1H), 7.45 (d, J = 8.4 Hz, 1H), 7.25-7.35 (m, 5H), 3.72 (t, J = 6.4 Hz, 2H),
2.95 (t, J = 6.4 Hz, 2H); MS m/z: 389 (M + 1).
5-Methyl-N-(6-pyrazin-2-ylpyridin-2-yl)pyridin-2-amine dihydrochloride ¹H NMR (400 MHz,
DMSO-d₆) δ 12.71 (s, 1H), 9.39 (s, 1H), 9.12 (d, J = 2.0 Hz, 1H), 8.89 (br s, 1H),
8.83 (s1, H), 8.55 (d, J = 6.0 Hz, 1H), 8.22 (s, 1H), 8.18 (s, 1H), 8.05 (dd, J = 2.0, 8.8 Hz,
1H), 7.95 (d, J = 6.0 Hz, 1H), 7.59 (d, J = 8.8 Hz, 1H), 2.34 (s, 1H); MS m/z: 264 (M + 1).
Ethyl 3-(6-{[6-(1,3-thiazol-2-yl)pyridin-2-yl]amino}pyridin-3-yl)propanoate
dihydrochloride ¹H NMR (400 MHz, DMSO-d₆) δ 12.54 (br s, 1H), 8.46 (s, 1H), 8.25 (dd,
J = 2.2 Hz, 8.8 Hz, 1H), 8.02-8.08 (m, 3H), 7.89 (d, J = 7.3 Hz, 1H), 7.84 (d, J = 8.8 Hz,
1H), 7.44 (d, J = 8.3 Hz, 1H), 4.07 (q, J = 7.3 Hz, 2H), 2.93 (t, J = 7.3 Hz, 2H), 2.74 (t, J = 7.3 Hz,
2H), 1.18 (t, J = 7.3 Hz, 3H),; MS m/z: 355 (M + 1).
3-Methoxy-N-pyridin-2-yl-6-(1,3-thiazol-2-yl)pyridin-2-amine dihydrochloride ¹H NMR
(400 MHz, DMSO-d₆) δ 10.87 (s, 1H), 8.51 (d, J = 5.9 Hz, 1H), 8.33-8.40 (m, 2H), 7.98 (d,
J = 3.4 Hz, 1H), 7.94 (d, J = 8.3 Hz, 1H), 7.88 (d, J = 2.9 Hz, 1H), 7.72 (d, J = 8.3 Hz, 1H),
7.40 (td, J = 5.8 Hz, 1.5 Hz, 1H), 4.07 (s, 3H); MS m/z: 285 (M + 1).
3-Methoxy-6-pyrazin-2-yl-N-pyridin-2-ylpyridin-2-amine dihydrochloride ¹H NMR (400 MHz,
DMSO-d₆) δ 11.16 (s, 1H), 9.46 (s, 1H), 8.80 (s, 1H), 8.72 (d, J = 2.4 Hz, 1H),
8.55 (d, J = 5.8 Hz, 1H), 8.33 (td, J = 8.7 Hz, 1.5 Hz, 1H), 8.20 (d, J = 8.7 Hz, 1H), 8.17 (d, J = 8.7 Hz,
1H), 7.80 (d, J = 8.3 Hz, 1H) 7.40 (t, J = 6.6 Hz, 1H), 4.07 (s, 3H); MS m/z: 280 (M + 1).
5-(1-Methylpiperidin-3-yl)-N-[6-(1,3-thiazol-2-yl)pyridin-2-yl]pyridin-2-amine
dihydrochloride ¹H NMR (400 MHz, DMSO-d₆) δ 11.88 (br s, 1H), 11.12 (br s, 1H),
8.36 (s, 1H), 7.94-8.14 (m, 5H), 8.27 (d, J = 7.3 Hz, 1H), 7.51 (d, J = 8.3 Hz, 1H),
3.39-3.56 (m, 2H), 3.13-3.37 (m, 2H), 2.87-3.00 (m, 1H), 2.76 (s, 1.5H), 2.75 (s, 1.5H),
1.91-2.25 (m, 3H), 1.61-1.74 (m, 1H); MS m/z: 351 (M + 1).
N-[6-(1,3-Thiazol-2-yl)pyridin-2-yl]-2,3′-bipyridin-6′-amine dihydrochloride ¹H NMR
(400 MHz, DMSO-d₆) δ 11.17 (br, 1H), 9.10 (d, J = 2.4 Hz, 1H), 8.78 (d, J = 4.9 Hz, 1H),
8.63-8.70 (m, 1H), 8.28 (br s, 1H), 8.15 (d, J = 8.8 Hz, 1H), 8.05 (d, J = 2.9 Hz, 1H),
7.94-8.01 (m, 2H), 7.80 (d, J = 7.3 Hz, 1H), 7.68 (br s, 1H), 7.66 (d, J = 8.3 Hz, 1H),; MS m/z:
332 (M + 1).
N-(6-Pyrazin-2-ylpyridin-2-yl)-2,3′-bipyridin-6′-amine dihydrochloride ¹H NMR (400 MHz,
DMSO-d₆) δ 12.41 (br, 1H), 9.65 (d, J = 1.4 Hz, 1H), 9.23 (s, 1H), 8.90-8.96 (m,
1H), 8.82-8.87 (m, 1H), 8.74-8.80 (m, 1H), 8.16-8.29 (m, 3H), 7.89 (d, J = 8.8 Hz1,
H), 7.70-7.75 (m, 1H), 7.60-7.66 (m, 1H); MS m/z: 327 (M + 1).
3-Methoxy-N-(5-piperidin-1-ylpyridin-2-yl)-6-(1,3-thiazol-2-yl)pyridin-2-amine
dihydrochloride ¹H NMR (400 MHz, DMSO-d₆) δ 10.05 (brs, 1H), 8.27-8.37 (m, 3H),
7.96 (d, J = 3.4 Hz, 1H), 7.85 (d, J = 3.0 Hz, 1H), 7.81 (d, J = 8.3 Hz, 1H), 7.60 (d, J = 8.8 Hz,
1H), 4.02 (s, 3H), 1.77 (brs, 4H), 1.61 (brs, 2H); MS m/z: 368 (M + 1).
3-Methoxy-N-(5-piperidin-1-ylpyridin-2-yl)-6-pyrazin-2-ylpyridin-2-amine
trihydrochloride ¹H NMR (400 MHz, DMSO-d₆) δ 10.67 (s, 1H), 9.47 (s, 1H), 8.83 (s,
1H), 8.72 (d, J = 2.5 Hz, 1H), 8.26-8.29 (m, 2H), 8.13 (d, J = 8.8 Hz, 1H), 8.09 (d, J = 9.8 Hz,
1H), 7.74 (d, J = 8.3 Hz, 1H), 4.07 (s, 3H), 1.76 (brs, 4H), 1.61 (brs, 2H); MS m/z:
363 (M + 1).
5-Methoxy-N-(5-piperidin-1-ylpyridin-2-yl)-2,2′-bipyridin-6-amine trihydrochloride ¹H
NMR (400 MHz, DMSO-d₆) δ 10.83 (s, 1H), 8.90 (d, J = 4.4 Hz, 1H), 8.47 (d, J = 8.3 Hz,
1H), 8.25-8.40 (m, 3H), 8.23 (d, J = 8.3 Hz, 1H), 8.01 (d, J = 9.3 Hz, 1H), 7.82 (d, J = 8.3 Hz,
1H), 7.77-7.80 (m, 1H), 4.10 (s, 3H), 1.79 (brs, 4H), 1.62 (brs, 2H); MS m/z: 362 (M + 1).
N-(5-Isopropylpyridin-2-yl)-3-methoxy-6-(1,3-thiazol-2-yl)pyridin-2-amine
dihydrochloride ¹H NMR (400 MHz, DMSO-d₆) δ 10.86 (s, 1H), 8.41-8.43 (m, 3H),
7.98 (d, J = 2.9 Hz, 1H), 7.90 (d, J = 8.8 Hz, 1H), 7.88 (d, J = 3.0 Hz, 1H), 7.60 (d, J = 8.8 Hz,
1H), 4.02 (s, 3H), 3.07 (t, J = 6.9 Hz, 1H), 1.27 (d, J = 6.9 Hz, 6H); MS m/z: 327 (M + 1).
N-(5-Isopropylpyridin-2-yl)-3-methoxy-6-pyrazin-2-ylpyridin-2-amine dihydrochloride ¹H
NMR (400 MHz, DMSO-d₆) δ 11.06 (s, 1H), 9.47 (d, J = 1.5 Hz, 1H), 8.80-8.82 (m, 1H),
8.73 (d, J = 2.5 Hz, 1H), 8.43 (d, J = 1.9 Hz, 1H), 8.36 (dd, J = 2.1 Hz, 9.3 Hz, 1H), 8.18 (d,
J = 8.8 Hz, 1H), 8.16 (d, J = 8.7 Hz, 1H), 7.78 (d, J = 8.8 Hz, 1H), 4.07 (s, 3H), 3.08 (sept,
J = 6.9 Hz, 1H), 1.26 (d, J = 6.9 Hz, 6H); MS m/z: 322 (M + 1).
N-(5-Isopropylpyridin-2-yl)-5-methoxy-2,2′-bipyridin-6-amine dihydrochloride ¹H NMR
(400 MHz, DMSO-d₆) δ 11.13 (s, 1H), 8.90 (d, J = 4.4 Hz, 1H), 8.49 (d, J = 7.8 Hz, 1H),
8.44 (d, J = 1.9 Hz, 1H), 8.37 (t, J = 7.4 Hz, 1H), 8.31 (dd, J = 2.1 Hz, 9.3 Hz, 1H), 8.25 (d,
J = 8.7 Hz, 1H), 8.11 (d, J = 9.3 Hz, 1H), 7.84 (d, J = 8.3 Hz, 1H), 7.77-7.82 (m, 1H),
4.10 (s, 3H), 3.07 (sept, J = 6.9 Hz, 1H), 1.28 (d, J = 6.9 Hz, 6H); MS m/z: 321 (M + 1).
3-Methoxy-N-(5-morpholin-4-ylpyridin-2-yl)-6-(1,3-thiazol-2-yl)pyridin-2-amine
trihydrochloride ¹H NMR (400 MHz, DMSO-d₆) δ 10.57 (s, 1H), 8.25-8.33 (m, 2H),
7.82 (d, J = 2.5 Hz, 1H), 7.97 (d, J = 3.4 Hz, 1H), 7.87 (d, J = 3.4 Hz, 1H), 7.85 (d, J = 8.8 Hz,
1H), 7.64 (d, J = 8.3 Hz, 1H), 4.02 (s, 3H), 3.79 (brt, J = 4.9 Hz, 4H), 3.24 (brt, J = 4.9 Hz,
4H); MS m/z: 370 (M + 1).
3-Methoxy-N-(5-morpholin-4-ylpyridin-2-yl)-6-pyrazin-2-ylpyridin-2-amine
trihydrochloride ¹H NMR (400 MHz, DMSO-d₆) δ 10.89 (s, 1H), 9.46 (s, 1H), 8.84 (s,
1H), 8.72 (d, J = 2.5 Hz, 1H), 8.12-8.18 (m, 2H), 8.06-8.08 (m, 2H), 7.74 (d, J = 8.8 Hz,
1H), 4.06 (s, 3H), 3.78 (brt, J = 4.9 Hz, 4H), 3.24 (brt, J = 4.9 Hz, 4H); MS m/z: 365 (M + 1).
5-Methoxy-N-(5-morpholin-4-ylpyridin-2-yl)-2,2′-bipyridin-6-amine trihydrochloride ¹H
NMR (400 MHz, DMSO-d₆) δ 10.93 (s, 1H), 8.92 (d, J = 4.8 Hz, 1H), 8.48 (d, J = 7.8 Hz,
1H), 8.32-8.42 (m, 1H), 8.19 (d, J = 8.3 Hz, 1H), 8.10 (d, J = 3.5 Hz, 1H), 8.06-8.11 (m,
1H), 7.98-8.03 (m, 1H), 7.80-7.85 (m, 1H), 7.79 (d, J = 8.8 Hz, 1H), 4.10 (s, 3H), 3.80 (brt,
J = 4.9 Hz, 4H), 3.23 (brt, J = 4.9 Hz, 4H); MS m/z: 364 (M + 1).
3-Methoxy-N-[5-(4-methylpiperazin-1-yl)pyridin-2-yl]-6-(1,3-thiazol-2-yl)pyridin-2-
amine dihydrochloride ¹H NMR (400 MHz, DMSO-d₆) δ 10.39 (s, 1H), 8.31 (d, J = 9.3 Hz,
1H), 8.14-8.18 (m, 1H), 8.10 (d, J = 2.5 Hz, 1H), 7.95 (d, J = 3.0 Hz, 1H), 7.83 (d, J = 2.9 Hz,
1H), 7.76 (d, J = 8.3 Hz, 1H), 7.56 (d, J = 8.3 Hz, 1H), 4.01 (s, 3H), 3.88 (d, J = 11.7 Hz,
2H), 3.53 (d, J = 11.7 Hz, 2H), 3.10-3.21 (m, 4H), 2.85 (d, J = 4.4 Hz, 3H); MS m/z: 383 (M + 1).
3-Methoxy-N-[5-(4-methylpiperazin-1-yl)pyridin-2-yl]-6-pyrazin-2-ylpyridin-2-amine
dihydrochloride ¹H NMR (400 MHz, DMSO-d₆) δ 11.50 (s, 1H), 10.81 (s, 1H), 9.48 (d, J = 1.5 Hz,
1H), 8.86-8.87 (m, 1H), 8.74 (d, J = 2.4 Hz, 1H), 8.12-8.19 (m, 3H), 8.05 (d, J = 9.8 Hz,
1H), 7.76 (d, J = 8.8 Hz, 1H), 4.07 (s, 3H), 3.92 (d, J = 12.7 Hz, 2H), 3.53 (d, J = 11.7 Hz,
2H), 3.28 (q, J = 12.7 Hz, 2H), 3.18-3.25 (m, 2H), 2.81 (d, J = 3.9 Hz, 3H); MS m/z:
378 (M + 1).
5-Methoxy-N-[5-(4-methylpiperazin-1-yl)pyridin-2-yl]-2,2′-bipyridin-6-amine
trihydrochloride ¹H NMR (400 MHz, DMSO-d₆) δ 11.49 (s, 1H), 8.93 (d, J = 4.8 Hz, 1H),
11.09 (s, 1H), 8.38 (d, J = 7.8 Hz, 1H), 8.24 (d, J = 2.4 Hz, 1H), 8.15 (d, J = 8.3 Hz, 2H),
8.00 (dd, J = 9.8 Hz, 2.9 Hz, 1H), 7.85 (t, J = 8.3 Hz, 2H), 7.65 (dd, J = 7.3 Hz, 4.9 Hz, 1H),
4.12 (s, 3H), 3.93 (d, J = 12.7 Hz, 2H), 3.53 (d, J = 11.7 Hz, 2H), 3.28 (q, J = 11.7 Hz, 2H),
3.15-3.25 (m, 2H), 2.82 (d, J = 4.9 Hz, 3H); MS m/z: 377 (M + 1).
N5-(2-Methoxyethyl)-N2-[3-methoxy-6-(1,3-thiazol-2-yl)pyridin-2-yl]-N5-
methylpyridine-2,5-diamine dihydrochloride ¹H NMR (400 MHz, DMSO-d₆) δ 10.38 (s,
1H), 8.32 (d, J = 9.8 Hz, 1H), 8.03 (dd, J = 9.8 Hz, 2.9 Hz, 1H), 7.95 (d, J = 3.4 Hz, 1H),
7.85 (d, J = 3.4 Hz, 1H), 7.76 (d, J = 8.3 Hz, 2H), 7.57 (d, J = 8.7 Hz, 1H), 4.00 (s, 3H),
3.61 (t, J = 4.7 Hz, 2H), 3.54 (t, J = 4.7 Hz, 2H), 3.27 (s, 3H), 3.00 (s, 3H); MS m/z: 372 (M + 1).
N-[3-Methoxy-6-(1,3-thiazol-2-yl)pyridin-2-yl]-2,3′-bipyridin-6′-amine dihydrochloride ¹H
NMR (400 MHz, DMSO-d₆) δ 10.31 (brs, 1H), 9.16 (d, J = 1.9 Hz, 1H), 8.91 (dd, J = 9.3 Hz,
1.9 Hz, 1H), 8.77 (d, J = 4.9 Hz, 1H), 8.52 (d, J = 9.3 Hz, 1H), 8.26 (d, J = 7.8 Hz, 1H),
8.19 (t, J = 7.8 Hz, 1H), 7.97 (d, J = 3.4 Hz, 1H), 8.90 (d, J = 8.8 Hz, 1H), 7.87 (d, J = 2.9 Hz,
1H), 7.68 (d, J = 8.3 Hz, 1H), 7.63 (t, J = 6.2 Hz, 1H), 4.04 (s, 3H); MS m/z: 362 (M + 1).
6-(5-Chloro-1,3-thiazol-2-yl)-N-pyridin-2-ylpyridin-2-amine hydrochloride ¹H NMR (400 MHz,
DMSO-d₆) δ 12.24 (brs, 1H), 8.50 (d, J = 5.6H, z1H), 8.27 (t, J = 7.8 Hz, 1H),
7.95 (t, J = 7.8 Hz, 1H), 8.03 (s, 1H), 7.88 (d, J = 8.8 Hz, 1H), 7.82 (d, J = 7.6 Hz, 1H), 7.49 (t, J = 8.0 Hz,
1H), 7.32 (t, J = 2.4 Hz, 1H); MS m/z: 289 (M + 1).
3-Methoxy-N-(5-pyrrolidin-1-ylpyridin-2-yl)-6-(1,3-thiazol-2-yl)pyridin-2-amine
trihydrochloride ¹H NMR (400 MHz, DMSO-d₆) δ 10.35 (s, 1H), 8.21 (d, J = 9.3 Hz, 1H),
7.96 (d, J = 3.4 Hz, 1H), 7.85 (d, J = 2.9 Hz, 1H), 7.82-7.85 (m, 1H), 7.81 (d, J = 8.3 Hz,
1H), 7.67 (d, J = 2.9 Hz, 1H), 7.61 (d, J = 8.3 Hz, 1H), 4.02 (s, 3H), 3.32 (s, 4H), 2.01 (s,
4H); MS m/z: 354 (M + 1).
1-(6-{[3-Methoxy-6-(1,3-thiazol-2-yl)pyridin-2-yl]amino}pyridin-3-yl)pyrrolidin-2-one
dihydrochloride ¹H NMR (400 MHz, DMSO-d₆) δ 10.56 (s, 1H), 8.85 (d, J = 1.9 Hz, 1H),
8.62 (dd, J = 9.8 Hz, 2.4 Hz, 1H), 8.41 (d, J = 9.8 Hz, 1H), 7.98 (d, J = 2.9 Hz, 1H), 7.89 (d,
J = 8.3 Hz, 1H), 7.87 (d, J = 3.4 Hz, 1H), 7.68 (d, J = 8.8 Hz, 1H), 4.03 (s, 3H), 3.91 (t, J = 7.6 Hz,
2H), 2.57 (t, J = 7.6 Hz, 2H), 2.14 (quintet, J = 7.6 Hz, 2H); MS m/z: 368 (M + 1).
6-(5-Isopropyl-1,3-thiazol-2-yl)-N-pyridin-2-ylpyridin-2-amine dihydrochloride ¹H NMR
(400 MHz, DMSO-d₆) δ 12.68 (brs, 1H), 8.54 (d, J = 6.4 Hz, 1H), 8.29 (t, J = 7.8 Hz, 1H),
8.05 (t, J = 8.3 Hz, 1H), 7.86 (d, J = 7.3 Hz, 1H), 7.85 (d J = 6.8 Hz, 1H), 7.80 (s, 1H),
7.46 (d, J = 8.3 Hz, 1H), 7.35 (t, J = 6.4 Hz, 1H), 3.33 (sep, J = 6.9 Hz, 1H), 1.38 (d, J = 6.9 Hz,
6H); MS m/z: 297 (M + 1).
1-(6-{[3-Methoxy-6-(1,3-thiazol-2-yl)pyridin-2-yl]amino}pyridin-3-yl)piperidin-2-one
dihydrochloride ¹H NMR (400 MHz, DMSO-d₆) δ 10.37 (brs, 1H), 8.53 (d, J = 2.0 Hz, 1H),
8.34 (d, J = 9.3 Hz, 1H), 8.26 (dd, J = 9.3 Hz, 2.4 Hz, 1H), 7.97 (d, J = 2.9 Hz, 1H), 7.88 (d,
J = 8.3 Hz, 1H), 7.86 (d, J = 2.9 Hz, 1H), 7.67 (d, J = 8.8 Hz, 1H), 4.03 (s, 3H), 3.71 (t, J = 6.5 Hz,
2H), 2.46 (t, J = 6.5 Hz, 2H), 1.85-1.95 (m, 4H); MS m/z: 382 (M + 1).
1-(6-{[3-Methoxy-6-(1,3-thiazol-2-yl)pyridin-2-yl]amino}pyridin-3-yl)piperidin-4-ol
trihydrochloride ¹H NMR (400 MHz, DMSO-d₆) δ 10.23 (brs, 1H), 8.31 (s, 2H), 8.19 (s,
1H), 7.96 (d, J = 3.4 Hz, 1H), 7.85 (d, J = 3.4 Hz, 1H), 7.83 (d, J = 8.3 Hz, 1H), 7.62 (d, J = 8.3 Hz,
1H), 4.02 (s, 3H), 3.75 (br quintet, J = 3.9 Hz, 1H), 3.57-3.65 (m, 2H), 3.12 (brt, J = 9.3 Hz,
2H), 1.87-1.97 (m, 2H), 1.52-1.66 (m, 2H); MS m/z: 384 (M + 1).
6-(5-Methyl-1,3-thiazol-2-yl)-N-pyridin-2-ylpyridin-2-amine dihydrochloride ¹H NMR
(400 MHz, DMSO-d₆) δ 12.77 (brs, 1H), 8.53 (d, J = 6.4 Hz, 1H), 8.30 (td, J = 8.3 Hz, 2.0 Hz,
1H), 8.05 (t, J = 8.3 Hz, 1H), 7.90 (d, J = 8.8 Hz, 1H), 7.83 (d, J = 7.3 Hz, 1H), 7.75 (d,
J = 1.0 Hz, 1H), 7.44 (d, J = 8.3 Hz, 1H), 7.34 (t, J = 6.3 Hz, 1H), 2.56 (d, J = 1.0 Hz, 3H);
MS m/z: 269 (M + 1).
6-(5-Chloro-1,3-thiazol-2-yl)-3-methoxy-N-pyridin-2-ylpyridin-2-amine dihydrochloride
¹H NMR (400 MHz, DMSO-d₆) δ 10.55 (brs, 1H), 8.52 (d, J = 4.4 Hz, 1H), 8.35 (td, J = 7.8 Hz,
2.0 Hz, 1H), 8.27 (d, J = 8.8 Hz, 1H), 7.98 (s, 1H), 7.85 (d, J = 8.3 Hz, 1H), 7.69 (d, J = 8.8 Hz,
1H), 7.38 (t, J = 6.3 Hz, 1H), 4.03 (s, 3H); MS m/z: 319 (M + 1).
6-(5-Ethyl-1,3-thiazol-2-yl)-N-pyridin-2-ylpyridin-2-amine dihydrochloride ¹H NMR (400 MHz,
DMSO-d₆) δ 12.72 (brs, 1H), 8.54 (d, J = 4.9 Hz, 1H), 8.30 (td, J = 8.3 Hz, 1.5 Hz,
1H), 8.06 (t, J = 7.8 Hz, 1H), 7.86 (t, J = 7.8 Hz, 2H), 7.79 (s, 1H), 7.44 (d, J = 8.3 Hz, 1H),
7.37 (t, J = 6.8 Hz, 1H), 2.95 (q, J = 7.3 Hz, 2H), 1.34 (t, J = 7.3 Hz, 3H); MS m/z: 283 (M + 1).
1-(6-{[6-(1H-Pyrazol-1-yl)pyridin-2-yl]amino}pyridin-3-yl)piperidin-2-one
monohydrochloride ¹H NMR (400 MHz, DMSO-d₆) δ 11.61 (br s, 1H), 8.65 (br s, 1H),
8.38 (br s, 1H), 8.74-8.78 (m, 1H), 7.85 (s, 1H), 8.02 (dd, J = 2.8, 9.2 Hz, 1H), 7.56 (m,
1H), 7.33-7.40 (m, 2H), 6.60 (br s, 1H), 3.67 (t, J = 5.2 Hz, 2H), 2.43 (t, J = 6.0 Hz, 2H),
1.83-1.93 (m, 4H); MS m/z: 335 (M + 1).
3-Methoxy-N-[5-(4-methoxypiperidin-1-yl)pyridin-2-yl]-6-(1,3-thiazol-2-yl)pyridin-2-
amine trihydrochloride ¹H NMR (400 MHz, DMSO-d₆) δ 10.34 (br s, 1H), 8.32 (br s, 2H),
8.16 (br s, 1H), 7.97 (d, J = 3.2 Hz, 1H), 7.86 (d, J = 3.2 Hz, 1H), 7.84 (d, J = 8.4 Hz, 1H),
7.63 (d, J = 8.4 Hz, 1H), 3.53-3.59 (m, 2H), 3.41-3.47 (m, 1H), 3.29 (s, 3H),
3.11-3.16 (m, 2H), 2.98-2.04 (m, 2H), 1.61-1.67 (m, 2H); MS m/z: 398 (M + 1).
N-[6-(5-Isopropyl-1,3-thiazol-2-yl)pyridin-2-yl]-5-morpholin-4-ylpyridin-2-amine
dihydrochloride ¹H NMR (400 MHz, DMSO-d₆) δ 12.19 (br s, 1H), 8.20 (dd, J = 2.8, 9.2 Hz,
1H), 7.96-8.01 (m, 2H), 7.84 (d, J = 9.2 Hz, 1H), 7.81 (s, 1H), 7.78 (d, J = 7.6 Hz,
1H), 7.34 (d, J = 8.4 Hz, 1H), 3.81 (t, J = 4.4 Hz, 4H), 3.32 (q, J = 6.8 Hz, 1H), 3.24 (t, J = 4.4 Hz,
4H), 1.38 (d, J = 6.8 Hz, 6H); MS m/z: 382 (M + 1).
N-[6-(5-Isopropyl-1,3-thiazol-2-yl)pyridin-2-yl]-5-piperidin-1-ylpyridin-2-amine
dihydrochloride ¹H NMR (400 MHz, DMSO-d₆) δ 11.16 (br s, 1H), 8.53 (br s, 1H),
8.31 (dd, J = 2.8, 9.2 Hz, 1H), 8.06 (d, J = 9.2 Hz, 1H), 7.91 (t, J = 8.4 Hz, 1H), 7.76 (s, 1H),
7.70 (d, J = 7.2 Hz, 1H), 7.46 (d, J = 8.4 Hz, 1H), 3.44 (br s, 4H), 3.32 (q, J = 6.8 Hz, 1H),
1.91 (br s, 4H), 1.64 (br s, 2H), 1.38 (d, J = 6.8 Hz, 6H); MS m/z: 380 (M + 1).
N-[6-(5-Isopropyl-1,3-thiazol-2-yl)pyridin-2-yl]-5-pyrrolidin-1-ylpyridin-2-amine
dihydrochloride ¹H NMR (400 MHz, DMSO-d₆) δ 12.10 (br s, 1H), 7.95 (t, J = 8.0 Hz,
1H), 7.77-7.82 (m, 3H), 7.73 (d, J = 7.6 Hz, 1H), 7.60 (br s, 1H), 7.27 (d, J = 8.0 Hz,
1H), 3.38 (q, J = 6.8 Hz, 1H), 2.31 (t, J = 6.4 Hz, 4H), 2.21 (t, J = 6.4 Hz, 4H), 1.38 (d, J = 6.8 Hz,
6H); MS m/z: 366 (M + 1).
1-(6-{[6-(5-Isopropyl-1,3-thiazol-2-yl)pyridin-2-yl]amino}pyridin-3-yl)pyrrolidin-2-one
dihydrochloride ¹H NMR (400 MHz, DMSO-d₆) δ 12.02 (br s, 1H), 8.42 (d, J = 2.4 Hz,
1H), 7.91-7.98 (m, 3H), 7.82 (br s, 1H), 7.76 (d, J = 7.6 Hz, 1H), 7.48 (d, J = 8.2 Hz,
1H), 3.42 (q, J = 6.8 Hz, 1H), 3.31 (t, J = 7.2 Hz, 2H), 2.18 (t, J = 7.2 Hz, 2H),
1.86-1.94 (m, 2H), 1.38 (d, J = 6.8 Hz, 6H); MS m/z: 380 (M + 1).
1-(6-{[6-(5-Isopropyl-1,3-thiazol-2-yl)pyridin-2-yl]amino}pyridin-3-yl)piperidin-2-one
dihydrochloride ¹H NMR (400 MHz, DMSO-d₆) δ 11.49 (br s, 1H), 8.41 (d, J = 2.4 Hz,
1H), 8.03 (dd, J = 2.4, 9.2 Hz, 1H), 7.93-7.97 (m, 2H), 7.74 (d, J = 7.6 Hz, 1H), 7.77 (br
s, 1H), 7.48 (d, J = 8.2 Hz, 1H), 3.68 (t, J = 6.6 Hz, 2H), 3.28-3.34 (m, 1H), 2.44 (t, J = 6.6 Hz,
2H), 2.18 (t, J = 6.6 Hz, 4H), 1.37 (d, J = 6.8 Hz, 6H); MS m/z: 394 (M + 1).
N-[6-(5-Chloro-1,3-thiazol-2-yl)pyridin-2-yl]-5-morpholin-4-ylpyridin-2-amine
dihydrochloride ¹H NMR (400 MHz, DMSO-d₆) δ 12.02 (br s, 1H), 8.17-8.23 (m, 1H),
8.07 (s, 1H), 8.04 (d, J = 2.8 Hz, 1H), 7.99 (t, J = 7.6 Hz, 1H), 7.83 (dd, J = 2.8, 9.6 Hz,
1H), 7.75 (d, J = 7.6 Hz, 1H), 7.36 (d, J = 8.0 Hz, 1H), 3.80 (t, J = 4.8 Hz, 4H), 3.24 (t, J = 4.8 Hz,
4H); MS m/z: 374 (M + 1).
N-[6-(5-Chloro-1,3-thiazol-2-yl)pyridin-2-yl]-5-piperidin-1-ylpyridin-2-amine
dihydrochloride ¹H NMR (400 MHz, DMSO-d₆) δ 11.07 (br s, 1H), 8.59 (br s, 1H),
8.34 (dd, J = 2.8, 9.6 Hz, 1H), 8.02-8.05 (m, 2H), 8.01 (t, J = 8.0 Hz, 1H), 7.66 (d, J = 7.2 Hz,
1H), 7.50 (d, J = 8.0 Hz, 1H), 3.46 (br s, 4H), 1.93 (br s, 4H), 1.65 (br s, 2H); MS m/z:
372 (M + 1).
N-[6-(5-Chloro-1,3-thiazol-2-yl)pyridin-2-yl]-5-pyrrolidin-1-ylpyridin-2-amine
dihydrochloride ¹H NMR (400 MHz, DMSO-d₆) δ 11.66 (br s, 1H), 7.97 (t, J = 8.0 Hz,
1H), 8.06 (s, 1H), 7.84 (dd, J = 2.8, 9.6 Hz, 1H), 7.69-7.76 (m, 3H), 7.26 (d, J = 8.0 Hz,
1H), 3.33 (br s, 4H), 2.02 (br s, 4H); MS m/z: 358 (M + 1).
1-(6-{[6-(5-Chloro-1,3-thiazol-2-yl)pyridin-2-yl]amino}pyridin-3-yl)pyrrolidin-2-one
dihydrochloride ¹H NMR (400 MHz, DMSO-d₆) δ 11.13 (br s, 1H), 8.70 (d, J = 2.4 Hz,
1H), 8.34 (dd, J = 2.4, 9.6 Hz, 1H), 8.04 (s, 1H), 7.98 (d, J = 9.2 Hz, 1H), 7.91 (d, J = 7.6 Hz,
1H), 7.68 (d, J = 7.6 Hz, 1H), 7.47 (d, J = 8.4 Hz, 1H), 3.88 (t, J = 6.8 Hz, 2H), 2.53 (t,
J = 6.8 Hz, 2H), 2.08-2.15 (m, 2H); MS m/z: 372 (M + 1).
1-(6-{[6-(5-Chloro-1,3-thiazol-2-yl)pyridin-2-yl]amino}pyridin-3-yl)piperidin-2-one
dihydrochloride ¹H NMR (400 MHz, DMSO-d₆) δ 10.93 (br s, 1H), 8.35 (d, J = 2.4 Hz,
1H), 8.03 (s, 1H), 7.89-7.95 (m, 3H), 7.67 (d, J = 8.0 Hz, 1H), 7.53 (d, J = 8.0 Hz, 1H),
3.66 (t, J = 6.8 Hz, 2H), 2.43 (t, J = 6.8 Hz, 2H), 1.82-1.93 (m, 4H); MS m/z: 386 (M + 1).
1-(6-{[6-(1,3-Thiazol-2-yl)pyridin-2-yl]amino}pyridin-3-yl)piperidin-2-one
trihydrochloride ¹H NMR (400 MHz, DMSO-d₆) δ 12.53 (br s, 1H), 8.53 (d, J = 2.4 Hz,
1H), 8.21 (dd, J = 2.4, 9.2 Hz, 1H), 8.07 (d, J = 2.8 Hz, 1H), 8.05 (d, J = 8.4 Hz, 1H),
8.00 (d, J = 2.8 Hz, 1H), 7.92 (d, J = 9.2 Hz, 1H), 7.86 (d, J = 7.2 Hz, 1H), 7.52 (d, J = 8.4 Hz,
1H), 3.71 (t, J = 6.4 Hz, 2H), 2.46 (t, J = 6.4 Hz, 2H), 1.85-1.93 (m, 4H); MS m/z:
352 (M + 1).
N⁵-(2-Methoxyethyl)-N⁵-methyl-N²-[6-(1,3-thiazol-2-yl)pyridin-2-yl]pyridine-2,5-diamine
trihydrochloride ¹H NMR (400 MHz, DMSO-d₆) δ 12.01 (br s, 1H), 7.97-8.07 (m, 4H),
7.74-7.82 (m, 3H), 7.31 (d, J = 8.4 Hz, 1H), 3.61 (t, J = 4.8 Hz, 2H), 3.53 (t, J = 4.8 Hz,
2H), 3.00 (s, 3H), 3.26 (s, 3H); MS m/z: 342 (M + 1).
1-(6-{[6-(1,3-Thiazol-2-yl)pyridin-2-yl]amino}pyridin-3-yl)piperidin-4-ol trihydrochloride
¹H NMR (400 MHz, DMSO-d₆) δ 11.40 (br s, 1H), 8.36 (br s, 1H), 8.27 (dd, J = 2.8, 9.2 Hz,
1H), 8.04 (d, J = 2.8 Hz, 1H), 7.93-7.98 (m, 3H), 7.78 (d, J = 7.2 Hz, 1H), 7.45 (d, J = 8.0 Hz,
1H), 3.80 (br s, 1H), 3.61 (br s, 2H), 3.22 (br s, 2H), 1.99 (br s, 2H), 1.67 (br s,
2H); MS m/z: 354 (M + 1).
5-(4-Methoxypiperidin-1-yl)-N-[6-(1,3-thiazol-2-yl)pyridin-2-yl]pyridin-2-amine
trihydrochloride ¹H NMR (400 MHz, DMSO-d₆) δ 11.78 (br s, 1H), 8.33 (br s, 1H),
8.29 (dd, J = 2.8, 9.6 Hz, 1H), 8.04 (d, J = 3.2 Hz, 1H), 7.93-7.98 (m, 3H), 7.79 (d, J = 8.0 Hz,
1H), 7.45 (d, J = 8.4 Hz, 1H), 3.54-3.61 (m, 2H), 3.46-3.51 (m, 1H), 3.31 (s, 3H),
3.21-3.28 (m, 2H), 2.08 (br s, 2H), 1.79 (br s, 2H); MS m/z: 368 (M + 1).
3-Methoxy-N-(5-phenylpyridin-2-yl)-6-(1,3-thiazol-2-yl)pyridin-2-amine dihydrochloride
¹H NMR (400 MHz, DMSO-d₆) δ 10.74 (br s, 1H), 8.89 (d, J = 2.4 Hz, 1H), 8.71 (dd, J = 2.4,
9.2 Hz, 1H), 8.49 (d, J = 9.2 Hz, 1H), 7.98 (d, J = 3.6 Hz, 1H), 7.92 (d, J = 8.4 Hz,
1H), 7.89 (d, J = 3.6 Hz, 1H), 7.84 (d, J = 7.2 Hz, 2H), 7.70 (d, J = 8.8 Hz, 1H), 7.55 (t, J = 7.2 Hz,
2H), 7.48 (t, J = 7.2 Hz, 1H), 4.04 (s, 3H); MS m/z: 361 (M + 1).
N-[6-(5-Chloro-1,3-thiazol-2-yl)pyridin-2-yl]-5-(4-methylpiperazin-1-yl)pyridin-2-amine
dihydrochloride ¹H NMR (400 MHz, DMSO-d₆) δ 11.33 (br s, 1H), 8.06-8.14 (m, 3H),
7.95 (t, J = 7.6 Hz, 1H), 7.89 (d, J = 9.2 Hz, 1H), 7.71 (d, J = 7.6 Hz, 1H), 7.40 (d, J = 8.4 Hz,
1H), 3.87 (d, J = 11.6 Hz, 2H), 3.52 (d, J = 11.6 Hz, 2H), 3.17-3.28 (m, 4H), 2.81 (s,
3H); MS m/z: 387 (M + 1).
N-[6-(5-Methyl-1,3-thiazol-2-yl)pyridin-2-yl]-5-piperidin-1-ylpyridin-2-amine
trihydrochloride ¹H NMR (400 MHz, DMSO-d₆) δ 11.26 (br s, 1H), 8.48 (br s, 1H),
8.30 (dd, J = 2.8, 9.6 Hz, 1H), 8.05 (br d, 1H), 7.91 (t, J = 8.0 Hz, 1H), 7.72 (s, 1H), 7.69 (d, J = 7.2 Hz,
1H), 7.43 (d, J = 8.0 Hz, 1H), 3.42 (br s, 4H), 2.55 (s, 3H), 1.90 (br s, 4H), 1.63 (br
s, 2H); MS m/z: 352 (M + 1).
5-(4-Methylpiperazin-1-yl)-N-[6-(5-methyl-1,3-thiazol-2-yl)pyridin-2-yl]pyridin-2-amine
trihydrochloride ¹H NMR (400 MHz, DMSO-d₆) δ 11.55 (br s, 1H), 8.23 (dd, J = 2.8, 9.6 Hz,
1H), 8.12 (d, J = 2.8 Hz, 1H), 8.01 (t, J = 8.0 Hz, 1H), 7.79 (d, J = 9.6 Hz, 1H), 7.76 (s,
1H), 7.37 (d, J = 8.8 Hz, 1H), 3.90 (d, J = 11.6 Hz, 2H), 3.53 (d, J = 11.6 Hz, 2H),
3.18-3.33 (m, 4H), 2.81 (s, 3H), 2.57 (s, 3H); MS m/z: 367 (M + 1).
N-[6-(5-Methyl-1,3-thiazol-2-yl)pyridin-2-yl]-5-pyrrolidin-1-ylpyridin-2-amine
dihydrochloride ¹H NMR (400 MHz, DMSO-d₆) δ 11.59 (br s, 1H) 7.96 (t, J = 8.0 Hz,
1H), 7.73-7.80 (m, 3H), 7.65-7.68 (m, 2H), 7.19 (d, J = 8.0 Hz, 1H), 3.33 (br s, 4H),
2.56 (s, 3H), 2.00 (br s, 4H); MS m/z: 338 (M + 1).
1-(6-{[6-(5-Methyl-1,3-thiazol-2-yl)pyridin-2-yl]amino}pyridin-3-yl)piperidin-2-one
trihydrochloride ¹H NMR (400 MHz, DMSO-d₆) δ 11.79 (br s, 1H), 8.46 (d, J = 2.0 Hz,
1H), 8.09 (dd, J = 2.0, 9.6 Hz, 1H), 7.96 (t, J = 7.6 Hz, 1H), 7.91 (d, J = 9.6 Hz, 1H),
7.75 (d, J = 7.6 Hz, 1H), 7.73 (s, 1H), 7.45 (d, J = 8.0 Hz, 1H), 3.71 (t, J = 6.0 Hz, 2H), 2.56 (s,
3H), 2.44 (t, J = 6.0 Hz, 2H) 1.84-1.94 (m, 4H); MS m/z: 366 (M + 1).
N²-[6-(5-Chloro-1,3-thiazol-2-yl)pyridin-2-yl]-N⁵-(2-methoxyethyl)-N⁵-methylpyridine-
2,5-diamine dihydrochloride ¹H NMR (400 MHz, DMSO-d₆) δ 11.73 (br s, 1H), 8.06 (s,
1H), 8.03 (dd, J = 2.8, 9.6 Hz, 1H), 7.96 (t, J = 8.0 Hz, 1H), 7.82 (br s, 1H), 7.78 (d, J = 9.6 Hz,
1H), 7.71 (d, J = 8.0 Hz, 1H), 7.29 (d, J = 8.0 Hz, 1H), 3.61 (t, J = 5.2 Hz, 2H), 3.53 (t,
J = 5.2 Hz, 2H), 3.25 (s, 3H), 3.00 (s, 3H); MS m/z: 376 (M + 1).
N⁵-(2-Methoxyethyl)-N⁵-methyl-N²-[6-(5-methyl-1,3-thiazol-2-yl)pyridin-2-yl]pyridine-
2,5-diamine trihydrochloride ¹H NMR (400 MHz, DMSO-d₆) δ 12.11 (br s, 1H), 8.05 (dd,
J = 2.8, 9.6 Hz, 1H), 7.96 (t, J = 8.4 Hz, 1H), 7.79 (br s, 1H), 7.77 (d, J = 9.6 Hz, 1H),
7.74 (s, 1H), 7.72 (d, J = 7.2 Hz, 1H), 7.28 (d, J = 8.4 Hz, 1H), 3.61 (t, J = 4.8 Hz, 2H), 3.53 (t,
J = 4.8 Hz, 2H), 3.00 (s, 3H), 3.25 (s, 3H), 2.56 (s, 3H); MS m/z: 356 (M + 1).
N²-[6-(5-Isopropyl-1,3-thiazol-2-yl)pyridin-2-yl]-N⁵-(2-methoxyethyl)-N⁵-methylpyridine-
2,5-diamine trihydrochloride ¹H NMR (400 MHz, DMSO-d₆) δ 12.16 (s, 1H), 8.06 (dd, J = 2.0,
9.6 Hz, 1H), 7.96 (t, J = 8.0 Hz, 1H), 7.81 (d, J = 9.6 Hz, 1H), 7.79 (s, 1H), 7.77 (d, J = 2.0 Hz,
1H), 7.74 (d, J = 8.0 Hz, 1H), 7.30 (d, J = 8.0 Hz, 1H), 3.61 (t, J = 4.8 Hz, 2H),
3.53 (t, J = 4.8 Hz, 2H), 3.28-3.36 (m, 1H), 3.26 (s, 3H), 3.01 (s, 3H), 1.37 (d, J = 6.8 Hz,
6H); MS m/z: 384 (M + 1).
N²-[6-(5-Chloro-1,3-thiazol-2-yl)-3-methoxypyridin-2-yl]-N⁵-(2-methoxyethyl)-N⁵-
methylpyridine-2,5-diamine trihydrochloride ¹H NMR (400 MHz, DMSO-d₆) δ 10.25 (br s,
1H), 8.22 (d, J = 9.6 Hz, 1H), 8.08 (dd, J = 2.8, 9.6 Hz, 1H), 7.95 (s, 1H), 7.83 (d, J = 2.8 Hz,
1H), 7.73 (d, J = 8.4 Hz, 1H), 7.58 (d, J = 8.4 Hz, 1H), 4.00 (s, 3H), 3.61 (t, J = 5.2 Hz,
2H), 3.53 (t, J = 5.2 Hz, 2H), 3.26 (s, 3H), 3.00 (s, 3H); MS m/z: 406 (M + 1).
6-(5-Chloro-1,3-thiazol-2-yl)-3-methoxy-N-(5-piperidin-1-ylpyridin-2-yl)pyridin-2-amine
dihydrochloride ¹H NMR (400 MHz, DMSO-d₆) δ 10.19 (s, 1H), 8.33 (dd, J = 2.4, 9.6 Hz,
1H), 8.27 (d, J = 9.6 Hz, 1H), 8.17 (br s, 1H), 7.95 (s, 1H), 7.75 (d, J = 8.4 Hz, 1H),
7.60 (d, J = 8.4 Hz, 1H), 4.01 (s, 3H), 3.52-3.60 (m, 2H), 3.41-3.47 (m, 1H), 3.30 (s, 3H),
3.10-3.18 (m, 2H), 1.96-2.04 (m, 2H), 1.60-1.68 (m, 2H); MS m/z: 432 (M + 1).
N-[5-(4-Isopropylpiperazin-1-yl)pyridin-2-yl]-3-methoxy-6-(1,3-thiazol-2-yl)pyridin-2-
amine trihydrochloride ¹H NMR (400 MHz, DMSO-d₆) δ 11.36-11.60 (m, 1H),
10.46-10.60 (m, 1H), 8.25-8.39 (m, 2H), 8.15-8.18 (m, 1H), 7.96-8.00 (m, 1H),
7.82-7.89 (m, 2H), 7.61-7.68 (m, 1H), 3.95-4.06 (m, 5H), 3.38-3.60 (m, 5H), 3.08-3.24 (m,
2H), 1.35 (d, J = 6.4 Hz, 6H); MS m/z: 411 (M + 1).
4-Methyl-1-(6-{[6-(1,3-thiazol-2-yl)pyridin-2-yl]amino}pyridin-3-yl)piperazin-2-one
dihydrochloride ¹H NMR (400 MHz, DMSO-d₆) δ 12.16 (1H, brs), 10.71 (1H, brs),
8.34 (1H, d, J = 3.0 Hz), 8.10 (1H, d, J = 8.8 Hz), 8.02 (1H, d, J = 3.0 Hz), 7.83-7.94 (3H, m),
7.72 (1H, d, J = 7.3 Hz), 7.58 (1H, d, J = 8.3 Hz), 3.50-5.00 (6H, m), 2.93 (3H, s); MS
m/z: 367 (M + 1).
1-(6-{[3-Methoxy-6-(1,3-thiazol-2-yl)pyridin-2-yl]amino}pyridin-3-yl)-4-methylpiperazin-
2-one tetrahydrochloride ¹H NMR (400 MHz, DMSO-d₆) δ 12.36 (brs, 1H), 9.91 (brs, 1H),
8.51 (d, J = 2.5 Hz, 1H), 8.45 (d, J = 9.3 Hz, 1H), 8.17 (dd, J = 9.3, 2.5 Hz, 1H), 7.97 (d, J = 3.4 Hz,
1H), 7.83-7.87 (m, 2H), 7.63 (d, J = 8.3 Hz, 1H), 3.40-4.30 (m, 9H), 2.94 (s, 3H);
MS m/z: 397 (M + 1).
6-(5-Chloro-1,3-thiazol-2-yl)-3-methoxy-N-(5-morpholin-4-ylpyridin-2-yl)pyridin-2-
amine dihydrochloride ¹H NMR (400 MHz, DMSO-d₆) δ 10.06 (brs, 1H), 8.16-8.24 (m,
2H), 8.03-8.06 (m, 1H), 7.96 (s, 1H), 7.74-7.79 (m, 1H), 7.61 (d, J = 8.3 Hz, 1H),
4.02 (s, 3H), 3.75-3.82 (m, 4H), 3.19-3.26 (m, 4H); MS m/z: 404 (M + 1).
6-(5-Chloro-1,3-thiazol-2-yl)-3-methoxy-N-(5-piperidin-1-ylpyridin-2-yl)pyridin-2-amine
dihydrochloride ¹H NMR (400 MHz, DMSO-d₆) δ 9.97 (brs, 1H), 8.14-8.36 (m, 3H),
7.94-7.97 (m, 1H), 7.69-7.79 (m, 1H), 7.53-7.64 (m, 1H), 4.01 (s, 3H), 3.24-3.42 (m,
4H), 1.53-1.82 (m, 6H); MS m/z: 402 (M + 1).
6-(5-Chloro-1,3-thiazol-2-yl)-3-methoxy-N-[5-(4-methylpiperazin-1-yl)pyridin-2-
yl]pyridin-2-amine dihydrochloride ¹H NMR (400 MHz, DMSO-d₆) δ 11.06 (brs, 1H),
9.72 (brs, 1H), 8.28 (d, J = 9.2 Hz, 1H), 8.06-8.18 (m, 2H), 7.97 (s, 1H), 7.74 (d, J = 8.3 Hz,
1H), 7.58 (d, J = 8.3 Hz, 1H), 4.01 (s, 3H), 3.42-3.60 (m, 4H), 3.15-3.28 (m, 4H),
2.77-2.88 (m, 3H); MS m/z: 417 (M + 1).
6-(5-Chloro-1,3-thiazol-2-yl)-3-methoxy-N-(5-pyrrolidin-1-ylpyridin-2-yl)pyridin-2-amine
dihydrochloride ¹H NMR (400 MHz, DMSO-d₆) δ 10.11 (brs, 1H), 8.14 (d, J = 9.3 Hz, 1H),
7.96 (s, 1H), 7.84 (dd, J = 9.8, 3.0 Hz, 1H), 7.74 (d, J = 8.3 Hz, 1H), 7.71 (d, J = 2.9 Hz,
1H), 7.59 (d, J = 8.3 Hz, 1H), 4.02 (s, 3H), 3.28-3.37 (m, 4H), 1.96-2.04 (m, 4H); MS
m/z: 388 (M + 1).
1-(6-{[6-(5-Chloro-1,3-thiazol-2-yl)-3-methoxypyridin-2-yl]amino}pyridin-3-yl)piperidin-
2-one dihydrochloride ¹H NMR (400 MHz, DMSO-d₆) δ 9.49 (brs, 1H), 8.43 (d, J = 2.4 Hz,
1H), 8.29 (d, J = 9.3 Hz, 1H), 8.12 (dd, J = 8.8, 2.4 Hz, 1H), 7.95 (s, 1H), 7.75 (d, J = 8.3 Hz,
1H), 7.59 (d, J = 8.3 Hz, 1H), 4.02 (s, 3H), 3.69 (t, J = 5.4 Hz, 2H), 2.44 (t, J = 6.4 Hz,
2H), 1.80-1.96 (m, 4H); MS m/z: 416 (M + 1).
1-(6-{[6-(5-Ethyl-1,3-thiazol-2-yl)pyridin-2-yl]amino}pyridin-3-yl)pyrrolidin-2-one
dihydrochloride ¹H NMR (400 MHz, DMSO-d₆) δ 11.64 (brs, 1H), 8.72 (d, J = 2.4 Hz, 1H),
8.38 (dd, J = 9.8 Hz, 2.4 Hz, 1H), 7.92-8.0 (m, 2H), 7.77 (s, 1H), 7.72 (d, J = 7.3 Hz, 1H),
7.43 (d, J = 8.3 Hz, 1H), 3.89 (t, J = 7.8 Hz, 2H), 2.95 (q, J = 7.3 Hz, 2H), 2.54 (t, J = 7.8 Hz,
2H), 2.12 (quintet, J = 7.8 Hz, 2H), 1.32 (t, J = 7.3 Hz, 3H); MS m/z: 366 (M + 1).
N-[6-(5-Ethyl-1,3-thiazol-2-yl)pyridine-2-yl]-5-morpholin-4-ylpyridin-2-amine
monohydrochloride ¹H NMR (400 MHz, DMSO-d₆) δ 11.54 (brs, 1H), 8.07 (brd, J = 9.3 Hz,
1H), 7.98 (d, J = 2.5 Hz, 1H), 7.95 (t, J = 8.3 Hz, 1H), 7.80 (d, J = 9.3 Hz, 1H), 7.77 (s,
1H), 7.73 (d, J = 7.9 Hz, 1H), 7.37 (d, J = 8.3 Hz, 1H), 3.79 (brt, J = 4.9 Hz, 4H), 3.20 (brt,
J = 4.9 Hz, 4H), 2.94 (q, J = 7.3 Hz, 2H), 1.33 (t, J = 7.3 Hz, 3H); MS m/z: 368 (M + 1).
3-Methoxy-N-[5-(4-methyl-1,4-diazepan-1-yl)pyridin-2-yl]-6-(1,3-thiazol-2-yl)pyridin-2-
amine tetrahydrochloride ¹H NMR (400 MHz, DMSO-d₆) δ 11.16-11.35 (br, 1H),
10.32-10.44 (br, 1H), 8.28-8.36 (m, 1H), 8.11 (dd, J = 3.5, 5.4 Hz, 1H), 7.98 (d, J = 2.9 Hz, 1H),
7.95 (d J = 2.9 Hz, 1H), 7.87 (d J = 3.5 Hz, 1H), 7.83 (d J = 8.3 Hz, 1H), 7.63 (d, J = 8.3 Hz,
1H), 4.03 (s, 3H), 3.80-3.96 (m, 2H), 3.40-3.60 (m, 4H), 3.10-3.30 (m, 2H), 2.79 (s, 1.5H),
2.78 (s, 1.5H), 2.36-2.48 (m, 1H), 2.10-2.24 (m, 1H); MS m/z: 397 (M + 1).
1-(6-{[6-(5-Chloro-1,3-thiazol-2-yl)pyridin-2-yl]amino}pyridin-3-yl)-4-methylpiperazin-2-
one dihydrochloride ¹H NMR (400 MHz, DMSO-d₆) δ 11.70-12.14 (br, 1H), 10.35 (s, 1H),
8.30 (d, J = 2.5 Hz, 1H), 8.05 (d, J = 9.3 Hz, 1H), 8.01 (s, 1H), 7.82-7.90 (m, 2H), 7.61 (d J = 7.3 Hz,
1H), 7.59 (d, J = 8.4 Hz, 1H), 3.95-4.25 (m, 3H), 3.73-3.95 (m, 2H), 3.50-3.70 (m,
1H), 2.93 (s, 3H); MS m/z: 401 (M + 1).
1-Methyl-4-(6-{[6-(1,3-thiazol-2-yl)pyridin-2-yl]amino}pyridin-3-yl)-1,4-diazepan-5-one
dihydrochloride ¹H NMR (400 MHz, DMSO-d₆) δ 11.40-11.64 (br, 1H), 10.42-10.62 (br,
1H), 8.28 (d, J = 2.4 Hz, 1H), 8.09 (d, 8.7 Hz, 1H), 8.01 (d, J = 2.9 Hz, 1H), 7.90 (d, J = 2.9 Hz,
1H), 7.87 (d, J = 7.8, 8.3 Hz, 1H), 7.81 (dd, J = 2.4, 8.7 Hz, 1H), 7.70 (d, J = 7.8 Hz,
1H), 7.53 (d, J = 8.3 Hz, 1H), 4.40-4.53 (m, 1H), 3.80-3.90 (m, 1H), 3.34-3.68 (m, 5H),
2.83 (s, 1.5H), 2.82 (s, 1.5H), 2.63-2.75 (m, 1H); MS m/z: 381 (M + 1).
1-Benzyl-4-(6-{[3-methoxy-6-(1,3-thiazol-2-yl)pyridin-2-yl]amino}pyridin-3-yl)-1,4-
diazepan-5-one dihydrochloride ¹H NMR (400 MHz, DMSO-d₆) δ 11.95 (brs, 1H),
9.40 (brs, 1H), 8.44 (d, J = 8.8 Hz, 1H), 8.40 (d, J = 2.4 Hz, 1H), 8.04 (dd, J = 9.3, 2.5 Hz1, H),
7.96 (d, J = 3.4 Hz, 1H), 7.84 (d, J = 2.9 Hz, 1H), 7.80 (d, J = 8.3 Hz, 1H), 7.65-7.72 (m,
2H), 7.58 (d, J = 8.3 Hz, 1H), 7.43-7.53 (m, 3H), 4.20-5.10 (m, 4H), 4.01 (s, 3H),
3.85-3.95 (m, 1H), 3.36-3.72 (m, 4H), 2.69-2.78 (m, 1H); MS m/z: 487 (M + 1).
tert-Butyl 3-oxo-4-(6-{[6-(1,3-thiazol-2-yl)pyridin-2-yl]amino}pyridin-3-yl)piperazine-1-
carboxylate ¹H NMR (400 MHz, DMSO-d₆) δ 10.02 (s, 1H), 8.27 (d, J = 2.5 Hz, 1H),
8.08 (d, J = 8.8 Hz, 1H), 7.99 (d, J = 3.4 Hz, 1H), 7.87 (d, J = 2.9 Hz, 1H), 7.78-7.85 (m, 1H),
7.76 (dd, J = 8.8, 2.9 Hz, 1H), 7.64 (d, J = 7.4 Hz1, H), 7.59 (d, J = 8.3 Hz, 1H), 4.09 (s,
2H), 3.65-3.79 (m, 4H), 1.45 (s, 9H); MS, m/z: 453 (M + 1).

### Example 15

### Preparation of 18 from 17

15.1 Stille Cross-Coupling

A mixture of 1.41 mmol of 17, 1.41 mmol of 14, and 0.07 mmol of Pd(PPh₃)₄ in 10 mL of toluene was stirred at 100° C. for 15 h under Ar. The reaction was quenched with 10 mL of saturated NaHCO₃. After the mixture was extracted with chloroform, the organic phase was washed with saturated NaCl, dried over MgSO₄, and concentrated under reduced pressure. The residue was purified by column chromatography to give 1.16 mmol of 18.

Most of 18 were converted to HCl salt by adding excess 4 M of HCl in 1,4-dioxane to a solution of 18 in MeOH. The pure salts were obtained by removing the solvents under reduced pressure or crystallizing in ethyl acetate.

15.2 Results

Analytical data for exemplary compounds of structure 18 are provided below.

15.2.a 4,6-Dipyridin-2-yl-3,4-dihydro-2H-pyrido[3,2-b][1,4]oxazine

¹H NMR (400 MHz, CDCl₃) δ 8.61 (d, J=4.8 Hz, 1H), 8.38 (d, J=4.8 Hz, 1H), 8.26 (d, J=8.4 Hz, 1H), 8.15 (d, J=8.4 Hz, 1H), 7.99 (d, J=8.4 Hz, 1H), 7.72 (t, J=7.6 Hz, 1H), 7.66 (t, J=7.6 Hz, 1H), 7.27 (d, J=8.4 Hz, 1H), 7.22 (t, J=4.8 Hz, 1H), 6.93 (t, J=4.8 Hz, 1H), 4.39 (t, J=4.4 Hz, 2H), 4.31 (t, J=4.4 Hz, 2H); MS m/z: 291 (M+1).

15.2.b (5-Nitro-2,2′-bipyridin-6-yl)(pyridin-2-yl)amine

¹H NMR (400 MHz, CDCl₃) δ 10.6 (s, 1H), 8.75 (d, J=4.4 Hz, 1H), 8.70 (d, J=8.8 Hz, 1H), 8.47 (d, J=8.0 Hz, 1H), 8.43 (d, J=4.4 Hz, 1H), 8.35 (d, J=8.0 Hz, 1H), 8.10 (d, J=8.8 Hz, 1H), 7.89 (t, J=8.0 Hz, 1H), 7.81 (t, J₁=8.0 Hz, J₂=4.4 Hz, 1H); MS m/z: 294 (M+1).

15.2.c N-[6-(Pyridin-2-ylamino)-2,2′-bipyridin-5-yl]acetamide

¹H NMR (400 MHz, CDCl₃) δ 8.68-8.69 (m, 1H), 8.51 (d, J=8.0 Hz, 1H), 8.47 (d, J=8.0 Hz, 1H), 8.35 (d, J=8.0 Hz, 1H), 8.23-8.27 (m, 1H), 7.65-7.80 (m, 4H), 7.30-7.35 (m, 1H), 7.05-7.09 (m, 1H), 2.17 (s, 3H); MS m/z: 306 (M+1).

15.2.d (5-Methoxy-2,2′-bipyridin-6-yl)(pyridin-2-yl)amine

¹H NMR (400 MHz, CDCl₃) δ 8.69 (d, J=8.4 Hz, 1H), 8.62 (m, 1H), 8.27-8.29 (m, 2H), 7.95 (d, J=8.4 Hz, 2H), 7.80 (m, 1H), 7.74 (m, 1H), 7.24 (dd, J=8.4 Hz, J₂=4.8 Hz, 1H), 7.13 (d, J=8.4 Hz, 1H), 6.90 (dd, J₁=8.4 Hz, J₂=4.8 Hz, 1H), 3.94 (s, 3H); MS m/z: 279 (M+1).

15.2.e Methyl 6-(pyridin-2-ylamino)-2,2′-bipyridine-5-carboxylate

¹H NMR (400 MHz, CDCl₃) δ 10.39 (br s, 1H), 8.72 (s, 1H), 8.69 (s, 1H), 8.44 (m, 1H), 8.42 (m, 1H), 8.36 (m, 1H), 8.24 (d, J=8.0 Hz, 1H), 7.83 (t, J=7.2 Hz, 1H), 7.75 (m, 1H), 7.34 (dd, J₁=8.4 Hz, J₂=5.2 Hz, 1H), 7.07 (dd, J₁=8.4 Hz, J₂=5.2 Hz, 1H), 4.33 (s, 3H); MS m/z: 307 (M+1).

15.2.f N,N-Dimethyl-6-(pyridin-2-ylamino)-2,2′-bipyridine-5-carboxamide

¹H NMR (400 MHz, CDCl₃) δ 8.67-8.69 (m, 2H), 8.47 (d, J=8.0 Hz, 1H), 8.29 (d, J=4.8 Hz, 1H), 7.99 (d, J=8.0 Hz, 1H), 7.85 (t, J=7.6 Hz, 1H), 7.71 (t, J=7.6 Hz, 1H), 7.67 (d, J=7.6 Hz, 1H), 7.35 (d, J=8.0 Hz, 1H), 7.33 (t, J=4.8 Hz, 1H), 6.91 (t, J=4.8 Hz, 1H), 3.12 (s, 3H), 3.11 (s, 3H); MS m/z: 320 (M+1).

15.2.g 5-Isopropoxy-N-pyridin-2-yl-2,2′-bipyridin-6-amine hydrochloride

¹H NMR (400 MHz, CDCl₃) δ 8.71 (d, J=8.4 Hz, 1H), 8.61-8.63 (m, 1H), 8.27-8.29 (m, 2H), 7.98 (br s, 1H), 7.94 (d, J=8.4 Hz, 1H), 7.76 (t, J=6.8 Hz, 1H), 7.72 (t, J=6.8 Hz, 1H), 7.17-7.23 (m, 1H), 7.13 (d, J=8.4 Hz, 1H), 6.88-6.91 (m, 1H), 4.63-4.71 (m, 6H), 1.42 (d, J=1.6 Hz, 1H), 1.41 (d, J=1.6 Hz, 1H); MS m/z: 307 (M+1).

15.2.h 5-(Benzyloxy)-N-pyridin-2-yl-2,2′-bipyridin-6-amine

¹H NMR (400 MHz, CDCl₃) δ 5.12 (s, 2H), 6.89 (dd, J₁=7.6 Hz, J₂=4.8 Hz, 1H), 7.17 (d, J=8.0 Hz, 1H), 7.20 (dd, J₁=7.6 Hz, J₂=4.8 Hz, 1H), 7.34-7.44 (m, 5H), 7.70-7.78 (m, 2H), 7.92 (d, J=8.0 Hz, 1H), 7.97 (br s, 1H), 8.24-8.28 (m, 2H), 8.61 (d, J=4.8 Hz, 1H), 8.68 (d, J=7.6 Hz, 1H); MS m/z: 355 (M+1).

15.2.i 5-(2-Methoxyethoxy)-N-pyridin-2-yl-2,2′-bipyridin-6-amine

¹H NMR (400 MHz, CDCl₃) δ 8.69 (d, J=8.0 Hz, 1H), 8.62-8.64 (m, 1H), 8.27-8.29 (m, 2H), 8.02 (br s, 1H), 7.94 (d, J=8.0 Hz, 1H), 7.80 (t, J=7.6 Hz, 1H), 7.73 (t, J=7.6 Hz, 1H), 7.23 (t, J=5.2 Hz, 1H), 7.17 (d, J=8.0 Hz, 1H), 6.91 (t, J=5.2 Hz, 1H), 4.25 (t, J=4.8 Hz, 2H), 3.81 (t, J=4.8 Hz, 2H), 3.46 (s, 3H); MS m/z: 323 (M+1).

15.2.j Methyl {[6-(pyridin-2-ylamino)-2,2′-bipyridin-5-yl]oxy}acetate

¹H NMR (400 MHz, CDCl₃) δ 9.20 (br s, 1H), 8.62 (d, J=4.8 Hz, 1H), 8.31-8.33 (m, 2H), 8.28 (d, J=7.6 Hz, 1H), 8.01 (d, J=8.0 Hz, 1H), 7.78 (t, J=7.6 Hz, 1H), 7.73 (t, J=7.6 Hz, 1H), 7.30 (d, J=8.0 Hz, 1H), 7.25 (t, J=4.8 Hz, 1H), 7.09 (t, J=4.8 Hz, 1H), 4.71 (s, 2H), 4.18 (s, 3H); MS m/z: 337 (M+1).

### Example 16

### Preparation of 18

16.1 Stannylation

17 (5 mmol, in 50 mL of dry THF) was added, through a canular, to a suspension of 6 mmol of KH (30% mineral oil) in 50 mL of dry THF at 0° C. under N₂. The resulting mixture was stirred for 30 min at 0° C. and then cooled to −78° C. To the cold solution was added 10.5 mmol of n-BuLi (2.5 M in hexane), and the mixture was stirred for 1 h before 10.5 mmol of Bu₃SnCl was added. The solution then was stirred for 2 h at −78° C. and allowed to warm to rt over 4 h before the reaction was quenched with 5 mL of isopropanol and 50 mL of water. After the mixture was diluted with 200 mL of ethyl acetate, the organic phase of the mixture was washed with saturated NaCl, dried over MgSO₄, and concentrated under reduced pressure. The crude product was purified by column chromatography to give 4.4 mmol of 19.

16.2 Results

Analytical data for exemplary compounds of structure 19 are provided below.

16.2.a (5-Chloro-pyridin-2-yl)-(6-tributylstannanyl-pyridin-2-yl)-amine

¹H NMR (300 MHz, DMSO-d₆) δ 9.71 (s, 1H), 8.18 (d, J=2.6 Hz, 1H), 8.10 (d, J=9.0 Hz, 1H), 7.62-7.58 (m, 1H), 7.43 (t, J=8.2 Hz, 1H), 7.28 (d, J=8.4 Hz, 1H), 6.90 (d, J=6.8 Hz, 1H), 1.62-1.45 (m, 6H), 1.42-1.19 (m, 6H), 1.17-0.94 (m, 6H), 0.91-0.72 (m, 9H); MS m/z: 496 (M+1).

16.2.b (5-Phenyl-pyridin-2-yl)-(6-tributylstannanyl-pyridin-2-yl)-amine

¹H NMR (300 MHz, DMSO-d₆) δ 9.66 (s, 1H), 8.52 (d, J=2.4 Hz, 1H), 8.13 (d, J=8.9 Hz, 1H), 7.87 (dd, J₁=8.7 Hz, J₂=2.5 Hz, 1H), 7.65-7.62 (m, 2H), 7.47-7.39 (m, 4H), 7.31 (d, J=7.3 Hz, 1H), 6.91 (d, J=6.5 Hz, 1H), 1.69-1.48 (m, 6H), 1.34-1.17 (m, 6H), 1.14-1.00 (m, 6H), 0.97-0.74 (m, 9H); MS m/z: 538 (M+1).

16.3 Synthesis of 18 from 19

A solution of 0.25 mmol of 19, 0.275 mmol of 12, and 0.025 mmol of Pd(PPh₃)₄ in 4 mL of dry DMF was refluxed for 1 day under N₂. The reaction was quenched with 2 mL of concentrated NH₄OH. After removal of DMF under reduced pressure, the residue was diluted with 100 mL of ethyl acetate and the organic mixture was washed with saturated NaCl, dried over MgSO₄, and concentrated under reduced pressure. The crude product was purified by column chromatography to give 0.12 mmol of 18.

Most of 18 were converted to HCl salt by adding excess 4 M of HCl in 1,4-dioxane to a solution of 18 in MeOH. The pure salts were obtained by removing the solvents under reduced pressure or crystallizing in ethyl acetate.

16.4 Results

Analytical data for exemplary compounds of structure 18 are provided below.

16.4.a (5-Chloro-pyridin-2-yl)-[6-(1-methyl-1H-imidazol-4-yl)-pyridin-2-yl]-amine.2HCl

¹H NMR (300 MHz, DMSO-d₆) δ 10.65 (s, 1H), 8.99 (s, 1H), 8.33 (d, J=2.4 Hz, 1H), 8.23 (d, J=1.3 Hz, 1H), 7.99 (d, J=8.9 Hz, 1H), 7.91 (d, J=8.7 Hz, 1H), 7.86 (dd, J₁=6.2 Hz, J₂=4.3 Hz, 1H), 7.52 (d, J=7.5 Hz, 1H), 7.39 (d, J=8.5 Hz, 1H), 3.90 (s, 3H); MS m/z: 286 (M+1).

16.4.b (5-Chloro-pyridin-2-yl)-(6-pyrazin-2-yl-pyridin-2-yl)-amine.2HCl

¹H NMR (300 MHz, DMSO-d₆) δ 10.71 (s, 1H), 9.53 (s, 1H), 8.80 (d, J=1.5 Hz, 1H), 8.75 (d, J=2.5 Hz, 1H), 8.39 (d, J=2.1 Hz, 1H), 7.98-7.94 (m, 2H), 7.93 (d, J=9.0 Hz, 1H), 7.85 (d, J=9.0 Hz, 1H), 7.60 (dd, J=6.4 Hz, J₂=2.9 Hz, 1H); MS m/z: 284 (M+1).

16.4.c (5-Phenyl-pyridin-2-yl)-(6-thiazol-2-yl-pyridin-2-yl)-amine.2HCl

¹H NMR (300 MHz, DMSO-d₆) δ 12.10 (s, 1H), 8.78 (d, J=1.8 Hz, 1H), 8.49 (dd, J₁=6.4 Hz, J₂=2.9 Hz, 1H); 8.05-7.97 (m, 4H), 7.83 (d, J=7.5 Hz, 1H), 7.78 (d, J=7.2 Hz, 2H), 7.55-7.40 (m, 4H); MS m/z: 331 (M+1).

### Example 17

### Preparation of 22

17.1 Synthesis of 21

To a solution of 25 mmol of 20 in 80 mL of dry THF at 0° C. was added 25 mmol of KH (30% mineral oil) under N₂. The suspension was stirred for 20 min before 10 mmol of 15 in 20 mL of dry THF was added over a period of 10 min. The resulting mixture was stirred for two days at 60° C. under N₂. The reaction was quenched dropwise with isopropanol (10 mL) and saturated NaCl (50 mL) at 0° C. and the mixture was diluted with 200 mL of ethyl acetate. After separating the two phases, the organic phase was washed with saturated NaCl, dried over MgSO₄, and concentrated under reduced pressure to give 10 mmol of 21.

17.2 Results

Analytical data for exemplary compounds of structure 21 are provided below.

17.2.a 2,6-Di-pyrazol-1-yl-pyridine

¹N NMR (300 MHz, DMSO-d₆) δ 8.92 (d, J=2.1 Hz, 2H), 8.11 (t, J=7.8 Hz, 1H), 7.84 (d, J=0.9 Hz, 2H), 7.79 (d, J=8.0 Hz, 2H), 6.61 (dd, J₁=2.8 Hz, J₂=0.9 Hz, 2H); MS m/z: 212 (M+1).

17.2.b 2,6-Bis-(4-methyl-pyrazol-1-yl)-pyridine

¹H NMR (300 MHz, DMSO-d₆) δ 8.67 (s, 2H), 8.04 (t, J=8.0 Hz, 1H), 7.69 (s, 2H), 7.66 (d, J=3.6 Hz, 2H), 2.12 (s, 6H); MS m/z: 240 (M+1).

17.3 Synthesis of 22 via Nucleophilic Replacement

To a solution of 1.66 mmol of 11 in 10 mL of anhydrous 1,4-dioxane was added 6.6 mmol of NaH (60% in mineral oil) followed by the addition of 1.66 mmol of 21, and the resulting mixture was stirred at 100° C. overnight under N₂. After the reaction was quenched with methanol, the solvents were removed. The residue was dissolved in 40 mL of ethyl acetate and the organic solution was washed with saturated NaCl, dried over MgSO₄, and concentrated under reduced pressure. The crude product was purified by column chromatography to give 0.8 mmol of 22.

17.4 Results

Analytical data for exemplary compounds of structure 22 are provided below.

17.4.a (5-Methoxy-pyridin-2-yl)-[6-(4-methyl-pyrazol-1-yl)-pyridin-2-yl]-amine.2HCl

¹H NMR (300 MHz, DMSO-d₆) δ 11.61 (s, 1H), 8.40 (s, 1H), 8.12 (d, J=3.0 Hz, 1H), 7.91 (d, J=8.0 Hz, 1H), 7.89 (dd, J₁=8.0 Hz, J₂=1.6 Hz, 1H), 7.77 (dd, J₁=5.7 Hz, J₂=3.5 Hz, 1H), 7.65 (s, 1H), 7.46 (d, J=7.9 Hz, 1H), 7.18 (d, J=8.0 Hz, 1H), 3.86 (s, 3H), 2.14 (s, 3H); MS m/z: 282 (M+1).

17.4.b [6-(4-Methyl-pyrazol-1-yl)-pyridin-2-yl]-(5-morpholin-4-yl-pyridin-2-yl)-amine.2HCl

¹H NMR (300 MHz, DMSO-d₆) δ 12.34 (s, 1H), 8.44 (s, 1H), 8.21 (dd, J=9.6 Hz, J₂=2.6 Hz, 1H), 7.96 (s, 1H), 8.04-7.92 (m, 1H), 7.50 (dd, J₁=9.6 Hz, J₂=3.7 Hz, 1H), 7.66 (s, 1H), 7.52 (d, J=4.0 Hz, 1H), 7.14 (d, J=8.2 Hz, 1H), 2.06 (s, 3H), 3.78-3.68 (m, 4H), 3.18-3.14 (m, 4H); MS m/z: 337 (M+1).

17.4.c [5-(3-Fluoro-phenyl)-pyridin-2-yl]-[6-(4-methyl-pyrazol-1-yl)-pyridin-2-yl]-amine.2HCl

¹H NMR (300 MHz, DMSO-d₆) δ 11.99 (s, 1H), 8.78 (s, 1H), 8.50 (d, J=9.2 Hz, 1H), 8.45 (s, 1H), 7.96 (t, J=8.0 Hz, 1H), 7.89 (d, J=8.8 Hz, 1H), 7.67 (s, 1H), 7.62 (d, J=9.3 Hz, 1H), 7.59-7.52 (m, 3H), 7.32 (d, J=8.0 Hz, 1H), 7.26 (t, J=8.2 Hz, 1H), 2.15 (s, 3H); MS m/z: 346 (M+1).

### Example 18

### Preparation of 22

18.1 Synthesis of 23

A mixture of 3.18 mmol of 15, 3.50 mmol of pyrazole 20, 0.32 mmol of Pd₂(dba)₃, 0.32 mmol of BINAP, and 4.77 mmol of Cs₂CO₃ in 30 mL of toluene was stirred at 80° C. for one day under Ar. The reaction mixture was diluted with 100 mL of chloroform and the organic solution was washed with saturated NaCl, dried over MgSO₄, and concentrated under reduced pressure. The residue was purified by column chromatography to give 1.36 mmol of 23.

18.2 Results

Analytical data for exemplary compounds of structure 23 are provided below.

18.2.a 6-Iodo-3-methoxy-2-(1H-pyrazol-1-yl)pyridine

¹H NMR (400 MHz, CDCl₃) δ 7.80 (d, J=2.0 Hz, 1H), 7.63 (d, J=8.8 Hz, 1H), 7.61 (d, J=2.0 Hz, 1H), 7.08 (d, J=8.8 Hz, 1H), 6.45 (t, J=2.0 Hz, 1H), 3.90 (s, 3H); MS m/z: 302 (M+1).

18.2.b 2-Bromo-6-(4-bromo-1H-pyrazol-1-yl)pyridine

¹H NMR (300 MHz, CDCl₃) δ 8.56 (s, 1H), 7.88 (d, J=8.0 Hz, 1H), 7.64-7.70 (m, 2H), 7.38 (d, J=8.0 Hz, 1H); MS m/z: 306 (M+1).

18.3 Synthesis of 22 via Nucleophilic Replacement

To a solution of 1.66 mmol of 11 in 10 mL of anhydrous 1,4-dioxane was added 6.6 mmol of NaH (60% in mineral oil) followed by the addition of 1.66 mmol of 23, and the resulting mixture was stirred at 100° C. overnight under N₂. After the reaction was quenched with methanol, the solvents were removed. The residue was dissolved in 40 mL of ethyl acetate and the organic solution was washed with saturated NaCl, dried over MgSO₄, and concentrated under reduced pressure. The crude product was purified by column chromatography to give 0.8 mmol of 22.

18.4 Results

Analytical data for exemplary compounds of structure 22 are provided below.

18.4.a 5-Methyl-N-[6-(1H-pyrazol-1-yl)pyridin-2-yl]pyridin-2-amine

¹H NMR (400 MHz, DMSO-d₆) δ 9.75 (s, 1H), 8.52 (d, J=3.0 Hz, 1H), 8.10 (s, 1H), 7.77-7.81 (m, 2H), 7.72 (d, J=8.3 Hz, 1H), 7.58 (dd, J₁=8.3 Hz, J₂=1.9 Hz, 1H), 7.52 (d, J=8.3 Hz, 1H), 7.33 (d, J=7.9 Hz, 1H), 6.58 (br t, J=1.9 Hz, 1H), 2.24 (s, 3H); MS m/z: 252 (M+1).

18.4.b Methyl 6-{[6-(1H-pyrazol-1-yl)pyridin-2-yl]amino}nicotinate monohydrochloride

¹H NMR (400 MHz, DMSO-d₆) δ 10.54 (s, 1H), 8.82 (d, J=2.4 Hz, 1H), 8.59 (d, J=2.4 Hz, 1H), 8.26 (dd, J₁=8.8 Hz, J₂=2.4 Hz, 1H), 7.92 (d, J=9.3 Hz, 1H), 7.90 (t, J=8.3 Hz, 1H), 7.82 (s, 1H), 7.60 (d, J=7.9 Hz, 1H), 7.49 (d, J=7.8 Hz, 1H), 6.60 (br t, J=1.5 Hz, 1H), 3.85 (s, 3H); MS m/z: 296 (M+1).

18.4.c 5-Methoxy-N-(5-morpholin-4-ylpyridin-2-yl)-6-(1H-pyrazol-1-yl)pyridin-2-amine dihydrochloride

¹H NMR (400 MHz, DMSO-d₆) δ 12.48 (s, 1H), 8.59 (d, J=2.8 Hz, 1H), 8.19 (dd, J₁=9.6 Hz, J₂=2.8 Hz, 1H), 7.98 (d, J=8.8 Hz, 1H), 7.94 (d, J=2.4 Hz, 1H), 7.77 (d, J=2.4 Hz, 1H), 7.60 (d, J=9.6 Hz, 1H), 7.28 (d, J=8.8 Hz, 1H), 6.63 (t, J=2.4 Hz, 1H), 3.98 (s, 3H), 3.77 (t, J=4.8 Hz, 4H), 3.18 (t, J=4.8 Hz, 4H); MS m/z: 353 (M+1).

18.4.d 4-Methyl-1-(6-{[6-(4-methyl-1H-pyrazol-1-yl)pyridin-2-yl]amino}pyridin-3-yl)piperazin-2-one dihydrochloride

¹H NMR (400 MHz, DMSO-d₆) δ 11.50-12.00 (br, 1H), 10.05-10.40 (br, 1H), 8.35 (s, 1H), 8.25-8.31 (m, 1H), 7.94 (d, J=8.7 Hz, 1H), 7.76-7.88 (m, 2H), 7.63 (s, 1H), 7.33-7.45 (m, 2H), 3.45-4.35 (m, 6H), 2.93 (s, 3H), 2.14 (s, 3H); MS m/z: 364 (M+1).

18.4.e 4-Methyl-1-(6-{[6-(1H-pyrazol-1-yl)pyridin-2-yl]amino}pyridin-3-yl)piperazin-2-one dihydrochloride

¹HNMR (400 MHz, DMSO-d₆) δ 11.70-12.15 (br, 1H), 10.30-10.60 (br, 1H), 8.57-8.62 (m, 1H), 8.28-8.34 (m, 1H), 7.80-7.94 (m, 4H), 7.42-7.50 (m, 2H), 6.57-6.62 (m, 1H), 3.95-4.50 (m, 3H), 3.70-3.95 (m, 2H), 3.50-3.70 (m, 1H), 2.92 (s, 3H); MS m/z: 350 (M+1).

18.4.f 1-(6-{[6-(4-Bromo-1H-pyrazol-1-yl)pyridin-2-yl]amino}pyridin-3-yl)-4-methylpiperazin-2-one dihydrochloride

¹HNMR (400 MHz, DMSO-d₆) δ 11.50-11.90 (br, 1H), 10.31 (s, 1H), 8.70 (s, 1H), 8.28 (d, J=2.4 Hz, 1H), 7.96 (s, 1H), 7.94 (d, J=9.3 Hz, 1H), 7.88 (dd, J₁=8.3 Hz, J₂=7.8 Hz, 1H), 7.83 (dd, J₁=9.3 Hz, J₂=2.4 Hz 1H), 7.49 (d, J=7.8 Hz, 1H), 7.40 (d, J=7.8 Hz, 1H), 3.70-4.23 (m, 6H), 2.93 (s, 3H); MS m/z: 430 (M+1).

### Example 19

### Preparation of 24

19.1 Reduction

To a solution of 2.2 mmol of LiAlH₄ in 4 mL of ether was added 0.74 mmol of 18 at 0° C. and stirred for 1 h. The reaction mixture was quenched with saturated Na₂SO₄, filtered through Celite, and washed with THF. The filtrate was dried over MgSO₄ and concentrated. The residue was purified by column chromatography on silica gel to give 0.25 mmol of 24.

19.2 Results

Analytical data for exemplary compounds of structure 24 are provided below.

19.2.a (6-{[6-(1,3-Thiazol-2-yl)pyridin-2-yl]amino}pyridin-3-yl)methanol

¹H NMR (400 MHz, DMSO-d₆) δ 9.86 (s, 1H), 8.21 (d, J=1.9 Hz, 1H), 8.00 (d, J=8.3 Hz, 1H), 7.98 (d, J=2.9 Hz, 1H), 7.85 (d, J=3.4 Hz, 1H), 7.80 (t, J=7.8 Hz, 1H), 7.70 (dd, J₁=8.6 Hz, J₂=2.2 Hz, 1H), 7.62 (s, 1H), 7.60 (s, 1H), 5.15 (t, J=5.8 Hz, 1H), 4.46 (d, J=5.8 Hz, 2H); MS m/z: 285 (M+1).

19.2.b 3-(6-{[6-(1,3-Thiazol-2-yl)pyridin-2-yl]amino}pyridin-3-yl)propan-1-ol dihydrochloride

¹H NMR (400 MHz, DMSO-d₆) δ 12.58 (s, 1H), 8.38 (s, 1H), 8.23 (dd, J=2.2 Hz, 8.8 Hz, 1H), 8.04-8.08 (m, 2H), 8.01 (d, J=3.5 Hz, 1H), 7.89 (d, J=7.3 Hz, 1H), 7.83 (d, J=8.8 Hz, 1H), 7.44 (d, J=7.8 Hz, 1H), 4.82 (br, 1H), 3.44 (t, J=6.3 Hz, 2H); MS m/z: 313 (M+1).

### Example 20

### Preparation of 25

20.1 Halogenation

A suspension of 10.1 mmol of 24 in 15 mL of SOCl₂ was stirred at rt for 30 min. The reaction mixture was concentrated in vacuo and the residue was diluted EtOH-AcOEt and preciptates were collected by filtration to give 6.5 mmol of 25.

20.2 Results

Analytical data for exemplary compound of structure 25 is provided below.

20.2.a 5-(Chloromethyl)-N-[6-(1,3-thiazol-2-yl)pyridin-2-yl]pyridin-2-amine dihydrochloride

¹H NMR (300 MHz, DMSO-d₆) δ 11.49 (br s, 1H), 8.47 (s, 1H), 8.18-7.89 (m, 5H), 7.91 (d, J=7.7 Hz, 1H), 7.55 (d, J=8.3 Hz, 1H), 4.85 (s, 2H); MS m/z: 304 (M+1).

### Example 21

### Preparation of 26

21.1 Nucleophilic Replacement

A solution of 0.61 mmol of 25 in 5 mL of DMF was added 3.0 mmol of a primary or secondary amine at rt and stirred for 20 min. The reaction mixture was concentrated in vacuo and the residue was diluted with AcOEt and water. The mixture was extracted with diluted HCl and the aqueous phase was made alkaline with K₂CO₃. The mixture was extracted with AcOEt and the organic phase was washed with brine, dried over MgSO₄, and concentrated in vacuo. The residue was purified with column chromatography on silica gel and converted into HCl salt to give 0.47 mmol of 26.

21.2 Results

Analytical data for exemplary compound of structure 26 are provided below.

### 21.1.a 5-(Pyrrolidin-1-ylmethyl)-N-[6-(1,3-thiazol-2-yl)pyridin-2-yl]pyridin-2-amine trihydrochloride

¹H NMR (400 MHz, DMSO-d₆) δ 12.14 (s, 1H), 11.78 (br s, 1H), 9.36 (d, J=1.9 Hz, 1H), 9.30 (br, 1H), 8.43 (dd, J=8.8 Hz, J₂=1.9 Hz, 1H), 7.96-8.08 (m, 4H), 7.86 (d, J=7.3 Hz, 1H), 7.59 (d, J=8.3 Hz, 1H), 4.42 (d, J=5.3 Hz, 2H), 3.35-3.47 (m, 2H), 3.02-3.15 (m, 2H), 1.84-2.10 (m, 2H); MS m/z: 338 (M+1).

21.2.b 5-(2-Pyrrolidin-1-ylethyl)-N-[6-(1,3-thiazol-2-yl)pyridin-2-yl]pyridin-2-amine trihydrochloride

Title compound was prepared from hydroxyethyl derivative by same method (Ex. 20 and Ex. 21). ¹H NMR (400 MHz, DMSO-d₆) δ 12.74 (s, 1H), 8.49 (d, J=2.4 Hz, 1H), 8.28 (dd, J₁=8.8 Hz, J₂=2.4 Hz, 1H), 8.06-8.09 (m, 2H), 8.02 (d, J=2.8 Hz, 1H), 7.91 (t, J=8.8 Hz, 2H), 7.49 (d, J=7.6 Hz, 1H), 3.53 (m, 2H), 3.45 (m, 2H), 3.16 (t, J=6.4 Hz, 2H), 3.06 (m, 2H), 2.03 (m, 2H), 1.92 (m, 2H); MS m/z: 352 (M+1).

21.2.c 5-[(Benzylamino)methyl]-N-[6-(1,3-thiazol-2-yl)pyridin-2-yl]pyridin-2-amine dihydrochloride

¹H NMR (400 MHz, DMSO-d₆) δ 11.28 (s, 1H), 9.93 (s, 1H), 8.53 (d, J=2.0 Hz, 1H), 8.24 (d, J=9.2 Hz, 1H), 8.23 (dd, J₁=8.5 Hz, J₂=2.5 Hz, 1H), 8.07 (d, J=3.4 Hz, 1H), 7.94-7.97 (m, 2H), 7.79 (d, J=7.3 Hz, 1H), 7.56-7.63 (m, 2H), 7.40-7.48 (m, 2H), 4.16-4.23 (m, 4H); MS m/z: 374 (M+1).

21.2.d 5-[(Cyclohexylamino)methyl]-N-[6-(1,3-thiazol-2-yl)pyridin-2-yl]pyridin-2-amine dihydrochloride

¹H NMR (400 MHz, DMSO-d₆) δ 9.42 (br s, 1H), 8.55 (d, J=1.9 Hz, 1H), 8.23 (d, J=8.8 Hz, 1H), 7.99-8.05 (m, 2H), 7.90-7.96 (m, 2H), 7.77 (d, J=7.3 Hz, 1H), 7.58 (d, J=8.3 Hz, 1H), 4.10-4.21 (m, 2H), 2.94-3.07 (m, 1H), 2.15 (d, J=9.8 Hz, 2H), 11.07 (br s, 1H), 1.79 (br d, J=11.7 Hz, 2H), 1.56-1.65 (m, 1H), 1.35-1.49 (m, 2H), 1.03-1.32 (m, 3H); MS m/z: 366 (M+1).

21.2.e 5-[(Isopropylamino)methyl]-N-[6-(1,3-thiazol-2-yl)pyridin-2-yl]pyridin-2-amine trihydrochloride

¹H NMR (400 MHz, DMSO-d₆) δ 11.76 (br s, 1H), 9.57 (br s, 1H), 8.63 (d, J=2.4 Hz, 1H), 8.37 (dd, J₁=8.8 Hz, J₂=2.4 Hz, 1H), 8.05 (d, J=2.8 Hz, 1H), 7.96-8.02 (m, 3H), 7.83 (d, J=6.8 Hz, 1H), 7.55 (d, J=8.0 Hz, 1H), 4.18 (t, J=6.0 Hz, 2H), 3.29-3.35 (m, 1H), 1.34 (d, J=6.4 Hz, 6H); MS m/z: 326 (M+1).

21.2.f 5-{[Cyclohexyl(methyl)amino]methyl}-N-[6-(1,3-thiazol-2-yl)pyridin-2-yl]pyridin-2-amine trihydrochloride

¹H NMR (400 MHz, DMSO-d₆) δ 11.04 (br s, 1H), 8.64 (d, J=2.0 Hz, 1H), 8.35 (dd, J₁=8.8 Hz, J₂=2.0 Hz, 1H), 8.04 (d, J=2.8 Hz, 1H), 7.98-8.01 (m, 2H), 7.97 (d, J=2.8 Hz, 1H), 7.81 (d, J=7.6 Hz, 1H), 7.61 (d, J=8.0 Hz, 1H), 4.41 to 4.45 (m, 1H), 4.21 to 4.26 (m, 1H), 3.20 (t, J=11.6 Hz, 1H), 2.59 (d, J=4.8 Hz, 3H), 2.19 (t, J=11.6 Hz, 2H), 1.84 (t, J=11.6 Hz, 2H), 1.12-1.63 (m, 6H); MS m/z: 380 (M+1).

21.2.g 5-[(tert-Butylamino)methyl]-N-[6-(1,3-thiazol-2-yl)pyridin-2-yl]pyridin-2-amine dihydrochloride

¹HNMR (400 MHz, DMSO-d₆) δ 11.58 (br s, 1H), 9.51 (br s, 1H), 8.63 (br s, 1H), 8.36 (t, J=8.4 Hz, 1H), 8.03 (d, J=3.2 Hz, 1H), 7.94-8.00 (m, 3H), 7.80 (dd, J₁=7.6 Hz, J₂=3.2 Hz, 1H), 7.52 (d, J=8.4 Hz, 1H), 4.14 (br s, 2H), 1.40 (s, 9H); MS m/z: 340 (M+1).

21.2.h 5-[(Cyclopentylamino)methyl]-N-[6-(1,3-thiazol-2-yl)pyridin-2-yl]pyridin-2-amine trihydrochloride

¹H NMR (400 MHz, DMSO-d₆) δ 11.83 (br s, 1H), 9.72 (br s, 1H), 8.63 (br s, 1H), 8.37 (d, J=9.2 Hz, 1H), 8.05 (d, J=3.2 Hz, 1H), 7.96-8.03 (m, 3H), 7.83 (d, J=7.2 Hz, 1H), 7.55 (d, J=8.0 Hz, 1H), 4.18 (t, J=5.6 Hz, 2H), 3.44-3.54 (m, 1H), 1.94-2.04 (m, 2H), 1.70-1.83 (m, 4H), 1.49-1.59 (m, 2H); MS m/z: 352 (M+1).

21.2.i 5-(3,4-Dihydroisoquinolin-2(1H)-ylmethyl)-N-[6-(1,3-thiazol-2-yl)pyridin-2-yl]pyridin-2-amine dihydrochloride

¹H NMR (400 MHz, DMSO-d₆) δ 11.85 (br s, 1H), 8.43 (s, 1H), 8.32 (d, J=8.8 Hz, 1H), 7.79-8.10 (m, 9H), 7.63 (d, J=8.4 Hz, 1H), 4.58 (s, 2H), 4.36 (s, 2H), 3.94 (t, J=8.0 Hz, 2H), 3.16 (t, J=8.0 Hz, 2H); MS m/z: 400 (M+1).

21.2.j 5-[(2,6-Dimethylpiperidin-1-yl)methyl]-N-[6-(1,3-thiazol-2-yl)pyridin-2-yl]pyridin-2-amine trihydrochloride

¹H NMR (400 MHz, DMSO-d₆) δ 11.58 (br s, 1H), 8.56 (s, 1H), 8.33 (d, J=8.8 Hz, 1H), 8.04 (d, J=3.2 Hz, 1H), 7.97-8.00 (m, 3H), 7.81 (d, J=7.6 Hz, 1H), 7.63 (d, J=8.0 Hz, 1H), 4.49 (s, 2H), 3.01 to 3.10 (m, 2H), 1.70-1.83 (m, 6H), 1.58 (d, J=6.0 Hz, 6H); MS m/z: 380 (M+1).

21.2.k 5-[(Diethylamino)methyl]-N-[6-(1,3-thiazol-2-yl)pyridin-2-yl]pyridin-2-amine dihydrochloride

¹H NMR (400 MHz, DMSO-d₆) δ 10.88 (br s, 1H), 10.82 (br s, 1H), 8.54 (s, 1H), 8.19 (d, 1H), 8.02-8.06 (m, 2H), 7.90-7.94 (m, 2H), 7.75 (d, 1H), 7.64 (d, 1H), 4.30 (d, 2H), 3.07 (m, 4H), 1.28 (s, 6H); MS m/z: 340 (M+1).

21.2.l 5-(Piperidin-1-ylmethyl)-N-[6-(1,3-thiazol-2-yl)pyridin-2-yl]pyridin-2-amine dihydrochloride

¹H NMR (400 MHz, DMSO-d₆) δ 11.17 (br s, 1H), 11.06 (br s, 1H), 8.55 (q, 1H), 8.23 (q, 1H), 8.02-8.04 (m, 2H), 7.93-7.97 (m, 2H), 7.78 (d, 1H), 7.63 (d, 1H), 4.28 (d, 2H), 3.34 (d, 2H), 2.84-2.89 (m, 2H), 1.80-1.92 (m, 4 h), 1.69-1.72 (m, 1H), 1.35-1.41 (m, 1H); MS m/z: 352 (M+1).

21.2.m 5-(Morpholin-4-ylmethyl)-N-[6-(1,3-thiazol-2-yl)pyridin-2-yl]pyridin-2-amine dihydrochloride

¹H NMR (400 MHz, DMSO-d₆) δ 11.75 (br s, 1H), 10.99 (br s, 1H), 8.53 (d, 1H), 8.20 (d, 1H), 8.02-8.05 (m, 2H), 7.91-7.95 (m, 2H), 7.76 (d, 1H), 7.64 (d, 1H), 4.35 (br s, 2H), 3.94-3.97 (m, 2H), 3.81 to 3.87 (m, 2H), 3.28-3.31 (m, 2H), 3.08-3.11 (m, 2H); MS m/z: 354 (M+1).

21.2.n 5-(3,6-Dihydropyridin-1 (2H)-ylmethyl)-N-[6-(1,3-thiazol-2-yl)pyridin-2-yl]pyridin-2-amine dihydrochloride

¹H NMR (400 MHz, DMSO-d₆) δ 11.31 (brs, 1H), 11.11 (brs, 1H), 8.57 (d, 1H), 8.24 (q, 1H), 8.03-8.07 (m, 2H), 7.92-7.96 (m, 2H), 7.77 (d, 1H), 7.63 (d, 1H), 5.90-5.92 (m, 1H), 5.70 (d, 1H), 4.32-4.42 (m, 2H), 3.61 (br s, 2H), 3.45-3.50 (m, 1H), 3.06-3.09 (m, 1H), 2.50-2.55 (m, 1H), 2.28-2.33 (br d, 1H); MS m/z: 350 (M+1).

21.2.o 5-(1,3-Dihydro-2H-isoindol-2-ylmethyl)-N-[6-(1,3-thiazol-2-yl)pyridin-2-yl]pyridin-2-amine dihydrochloride

¹H NMR (400 MHz, DMSO-d₆) δ 12.58 (br s, 1H), 11.35 (br s, 1H), 8.67 (s, 1H), 8.36 (d, 1H), 8.03-8.05 (m, 2H), 7.95-7.99 (m, 2H), 7.80 (d, 1H), 7.64 (d, 1H), 7.35-7.41 (m, 4H), 4.65 (br s, 6H); MS m/z: 386 (M+1).

21.2.p N-[(6-{[6-(1,3-Thiazol-2-yl)pyridin-2-yl]amino}pyridin-3-yl)methyl]pyrazin-2-amine dihydrochloride

¹H NMR (400 MHz, DMSO-d₆) δ 12.32 (br s, 1H), 8.44 (s, 1H), 8.25 (d, 1H), 8.15 (s, 1H), 7.99-8.07 (m, 4H), 7.83-7.89 (m, 2H), 7.78 (d, 1H), 7.45 (d, 1H), 4.58 (s, 2H); MS m/z: 362 (M+1).

21.2.q N-[(6-{[6-(1,3-Thiazol-2-yl)pyridin-2-yl]amino}pyridin-3-yl)methyl]pyrimidin-2-amine dihydrochloride

¹H NMR (400 MHz, DMSO-d₆) δ 12.25 (br s, 1H), 8.48 (d, 2H), 8.43 (s, 1H), 8.24 (d, 1H), 7.98-8.06 (m, 3H), 7.86-7.89 (m, 2H), 7.47 (d, 1H), 6.81-6.84 (m, 1H), 4.64 (s, 2H); MS m/z: 362 (M+1).

21.2.r 5-[(Ethylamino)methyl]-N-[6-(1,3-thiazol-2-yl)pyridin-2-yl]pyridin-2-amine dihydrochloride

¹H NMR (400 MHz, DMSO-d₆) δ 11.09 (br s, 1H), 9.46 (br s, 2H), 8.52 (s, 1H), 8.20 (d, J=8.3 Hz, 1H), 7.92-8.04 (m, 1H), 7.77 (d, J=7.8 Hz, 1H), 7.60 (d, J=8.3 Hz, 1H), 2.98-3.20 (m, 2H), (br, 2H), 1.26 (t, J=7.3 Hz, 3H); MS m/z: 312 (M+1).

21.2.s 5-[(4-Phenyl-3,6-dihydropyridin-1(2H)-ylmethyl]-N-[6-(1,3-thiazol-2-yl)pyridin-2-yl]pyridin-2-amine dihydrochloride

¹HNMR (400 MHz, DMSO-d₆) δ11.41 (brs, 1H), 11.11 (brs, 1H), 8.69 (s, 1H), 8.27 (d, J=8.6 Hz, 1H), 7.91-8.15 (m, 4H), 7.77 (d, J=7.5 Hz, 1H), 7.64 (d, J=8.0 Hz, 1H), 7.30-7.50 (m, 5H), 6.18 (s, 1H), 4.40-4.52 (m, 2H), 3.82 (s, 2H), 3.55-3.65 (m, 1H), 3.20-3.30 (m, 1H), 2.70-3.02 (m, 2H); MS m/z: 426 (M+1).

### Example 21A

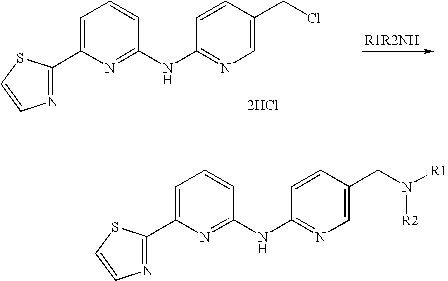

A mixture of 0.025 mmol of 5-(chloromethyl)-N-[6-(1,3-thiazol-2-yl)pyridin-2-yl]pyridin-2-amine.2HCl, 0.05 mmol of amine and 0.05 mmol of polystylene-bound diisopropyletylamine (PS-DIEA) in 1 mL of DMF was stirred at room temperature overnight.

To the solution was added 0.05 mmol of PS-NCO and stirred for 1 hour. The reaction mixture was filtered and concentrated under a reduced pressure. The residue was purified by preparative LC-MS to yield the product.

HPLC condition: Wakosil-II 5C18 AR 4.6*30 mm MeOH/5 mM TFA-H₂O=10/90 (0 min)−100/0 (4.0 min)−100/0 (4.5 min), MS: ESI(+).

Characterization data for some modulators of the present invention are presented in Table 2 below.

TABLE 2

| | RETENTION | |
|---|---|---|
| | TIME | |
| Name | (min) | OBS MASS |
| 5-[(Benzylamino)methyl]-N-[6-(1,3-thiazol-2-yl)pyridin-2- | 1.43 | 374 |
| yl]pyridin-2-amine | | |
| 5-{[(2-Fluorobenzyl)amino]methyl}-N-[6-(1,3-thiazol-2- | 1.45 | 392 |
| yl)pyridin-2-yl]pyridin-2-amine | | |
| 5-{[(2-Methoxybenzyl)amino]methyl}-N-[6-(1,3-thiazol-2- | 1.59 | 404 |
| yl)pyridin-2-yl]pyridin-2-amine | | |
| 5-{[(3-Fluorobenzyl)amino]methyl}-N-[6-(1,3-thiazol-2- | 1.49 | 392 |
| yl)pyridin-2-yl]pyridin-2-amine | | |
| 5-{[(3-Methoxybenzyl)amino]methyl}-N-[6-(1,3-thiazol-2- | 1.56 | 404 |
| yl)pyridin-2-yl]pyridin-2-amine | | |
| 5-{[(4-Fluorobenzyl)amino]methyl}-N-[6-(1,3-thiazol-2- | 1.56 | 392 |
| yl)pyridin-2-yl]pyridin-2-amine | | |
| 5-{[(4-Methoxybenzyl)amino]methyl}-N-[6-(1,3-thiazol-2- | 1.53 | 404 |
| yl)pyridin-2-yl]pyridin-2-amine | | |
| 5-{[(1,3-Benzodioxol-5-ylmethyl)amino]methyl}-N-[6-(1,3- | 1.46 | 418 |
| thiazol-2-yl)pyridin-2-yl]pyridin-2-amine | | |
| 5-{[(2-Furanylmethyl)amino]methyl}-N-[6-(1,3-thiazol-2- | 1.22 | 364 |
| yl)pyridin-2-yl]pyridin-2-amine | | |
| N-[6-(1,3-Thiazol-2-yl)pyridin-2-yl]-5-{[(2- | 1.33 | 380 |
| thienylmethyl)amino]methyl}pyridin-2-amine | | |
| 5-{[(Pyridin-3-ylmethyl)amino]methyl}-N-[6-(1,3-thiazol-2- | 0.91 | 375 |
| yl)pyridin-2-yl]pyridin-2-amine | | |
| 5-{[(2-Phenylethyl)amino]methyl}-N-[6-(1,3-thiazol-2- | 1.6 | 388 |
| yl)pyridin-2-yl]pyridin-2-amine | | |
| 5-({[2-(2-Fluorophenyl)ethyl]amino}methyl)-N-[6-(1,3- | 1.65 | 406 |
| thiazol-2-yl)pyridin-2-yl]pyridin-2-amine | | |
| 5-({[2-(2-Methoxyphenyl)ethyl]amino}methyl)-N-[6-(1,3- | 1.75 | 418 |
| thiazol-2-yl)pyridin-2-yl]pyridin-2-amine | | |
| 5-({[2-(3-Fluorophenyl)ethyl]amino}methyl)-N-[6-(1,3- | 1.66 | 406 |
| thiazol-2-yl)pyridin-2-yl]pyridin-2-amine | | |
| 5-({[2-(3-Methoxyphenyl)ethyl]amino}methyl)-N-[6-(1,3- | 1.66 | 418 |
| thiazol-2-yl)pyridin-2-yl]pyridin-2-amine | | |
| 5-({[2-(4-Fluorophenyl)ethyl]amino}methyl)-N-[6-(1,3- | 1.68 | 406 |
| thiazol-2-yl)pyridin-2-yl]pyridin-2-amine | | |
| 5-({[2-(4-Methoxyphenyl)ethyl]amino}methyl)-N-[6-(1,3- | 1.62 | 418 |
| thiazol-2-yl)pyridin-2-yl]pyridin-2-amine | | |
| N-[6-(1,3-Thiazol-2-yl)pyridin-2-yl]-5-({[2-(2- | 1.49 | 394 |
| thienyl)ethyl]amino}methyl)pyridin-2-amine | | |
| 5-({[2-(1H-Indol-3-yl)ethyl]amino}methyl)-N-[6-(1,3-thiazol- | 1.62 | 427 |
| 2-yl)pyridin-2-yl]pyridin-2-amine | | |
| 5-{[(2-Pyridin-2-ylethyl)amino]methyl}-N-[6-(1,3-thiazol-2- | 0.53 | 389 |
| yl)pyridin-2-yl]pyridin-2-amine | | |
| 5-{[(2-Pyridin-3-ylethyl)amino]methyl}-N-[6-(1,3-thiazol-2- | 0.63 | 389 |
| yl)pyridin-2-yl]pyridin-2-amine | | |
| 5-{[(2-Pyridin-4-ylethyl)amino]methyl}-N-[6-(1,3-thiazol-2- | 0.61 | 389 |
| yl)pyridin-2-yl]pyridin-2-amine | | |
| 5-{[(3-Phenylpropyl)amino]methyl}-N-[6-(1,3-thiazol-2- | 1.76 | 402 |
| yl)pyridin-2-yl]pyridin-2-amine | | |
| 5-({[3-(1H-Imidazol-1-yl)propyl]amino}methyl)-N-[6-(1,3- | 0.66 | 392 |
| thiazol-2-yl)pyridin-2-yl]pyridin-2-amine | | |
| 5-{[(4-Phenylbutyl)amino]methyl}-N-[6-(1,3-thiazol-2- | 1.94 | 416 |
| yl)pyridin-2-yl]pyridin-2-amine | | |
| 5-({[2-(1H-Benzimidazol-2-yl)ethyl]amino}methyl)-N-[6- | 1.57 | 428 |
| (1,3-thiazol-2-yl)pyridin-2-yl]pyridin-2-amine | | |
| 5-[(Propylamino)methyl]-N-[6-(1,3-thiazol-2-yl)pyridin-2- | 1.06 | 326 |
| yl]pyridin-2-amine | | |
| 5-[(Isopropylamino)methyl]-N-[6-(1,3-thiazol-2-yl)pyridin-2- | 1 | 326 |
| yl]pyridin-2-amine | | |
| 5-[(tert-Butylamino)methyl]-N-[6-(1,3-thiazol-2-yl)pyridin-2- | 1.09 | 340 |
| yl]pyridin-2-amine | | |
| 5-{[(3-Methylbutyl)amino]methyl}-N-[6-(1,3-thiazol-2- | 1.49 | 354 |
| yl)pyridin-2-yl]pyridin-2-amine | | |
| 5-[(Pentylamino)methyl]-N-[6-(1,3-thiazol-2-yl)pyridin-2- | 1.54 | 354 |
| yl]pyridin-2-amine | | |
| 5-{[(1-Methylhexyl)amino]methyl}-N-[6-(1,3-thiazol-2- | 1.9 | 382 |
| yl)pyridin-2-yl]pyridin-2-amine | | |
| 5-{[(1-Propylbutyl)amino]methyl}-N-[6-(1,3-thiazol-2- | 1.8 | 382 |
| yl)pyridin-2-yl]pyridin-2-amine | | |
| 5-[(Cyclopentylamino)methyl]-N-[6-(1,3-thiazol-2-yl)pyridin- | 1.23 | 352 |
| 2-yl]pyridin-2-amine | | |
| N,N-Dimethyl-N′-[(6-{[6-(1,3-thiazol-2-yl)pyridin-2- | 0.72 | 395 |
| yl]amino}pyridin-3-yl)methyl]cyclopentane-1,2-diamine | | |
| 5-({[2-Pyrrolidin-1-ylcyclopentyl]amino}methyl)-N-[6-(1,3- | 0.88 | 421 |
| thiazol-2-yl)pyridin-2-yl]pyridin-2-amine | | |
| 5-({[2-Piperidin-1-ylcyclopentyl]amino}methyl)-N-[6-(1,3- | 1.15 | 435 |
| thiazol-2-yl)pyridin-2-yl]pyridin-2-amine | | |
| 5-({[2-(4-Methylpiperazin-1-yl)cyclopentyl]amino}methyl)- | 0.95 | 450 |
| N-[6-(1,3-thiazol-2-yl)pyridin-2-yl]pyridin-2-amine | | |
| 5-({[2-Morpholin-4-ylcyclopentyl]amino}methyl)-N-[6-(1,3- | 1.48 | 437 |
| thiazol-2-yl)pyridin-2-yl]pyridin-2-amine | | |
| 3-{[(6-{[6-(1,3-Thiazol-2-yl)pyridin-2-yl]amino}pyridin-3- | 1.02 | 368 |
| yl)methyl]amino}dihydrofuran-2(3H)-one | | |
| 5-({[(3R)-1-Benzylpyrrolidin-3-yl]amino}methyl)-N-[6-(1,3- | 1.38 | 443 |
| thiazol-2-yl)pyridin-2-yl]pyridin-2-amine | | |
| 5-[(Cyclohexylamino)methyl]-N-[6-(1,3-thiazol-2-yl)pyridin- | 1.42 | 366 |
| 2-yl]pyridin-2-amine | | |
| 5-({[2-Pyrrolidin-1-ylcyclohexyl]amino}methyl)-N-[6-(1,3- | 1.41 | 435 |
| thiazol-2-yl)pyridin-2-yl]pyridin-2-amine | | |
| 5-({[2-Piperidin-1-ylcyclohexyl]amino}methyl)-N-[6-(1,3- | 1.65 | 449 |
| thiazol-2-yl)pyridin-2-yl]pyridin-2-amine | | |
| 5-({[2-(4-Methylpiperazin-1-yl)cyclohexyl]amino}methy)-N- | 1.12 | 464 |
| [6-(1,3-thiazol-2-yl)pyridin-2-yl]pyridin-2-amine | | |
| 5-({[2-Morpholin-4-ylcyclohexyl]amino}methyl)-N-[6-(1,3- | 1.63 | 451 |
| thiazol-2-yl)pyridin-2-yl]pyridin-2-amine | | |
| trans-4-{[(6-{[6-(1,3-Thiazol-2-yl)pyridin-2- | 1.22 | 382 |
| yl]amino}pyridin-3-yl)methyl]amino}cyclohexanol | | |
| 5-{[(4-tert-Butylcyclohexyl)amino]methyl}-N-[6-(1,3-thiazol- | 2.22 | 422 |
| 2-yl)pyridin-2-yl]pyridin-2-amine | | |
| N,N-Dimethyl-N′-[(6-{[6-(1,3-thiazol-2-yl)pyridin-2- | 1.32 | 407 |
| yl]amino}pyridin-3-yl)methyl]cyclohex-4-ene-1,2-diamine | | |
| 5-{[(1-Benzylpiperidin-4-yl)amino]methyl}-N-[6-(1,3-thiazol- | 1.33 | 457 |
| 2-yl)pyridin-2-yl]pyridin-2-amine | | |
| Ethyl 4-{[(6-{[6-(1,3-Thiazol-2-yl)pyridin-2- | 1.4 | 439 |
| yl]amino}pyridin-3-yl)methyl]amino}piperidine-1- | | |
| carboxylate | | |
| 5-[(Cycloheptylamino)methyl]-N-[6-(1,3-thiazol-2-yl)pyridin- | 1.6 | 380 |
| 2-yl]pyridin-2-amine | | |
| 5-[(Cyclooctylamino)methyl]-N-[6-(1,3-thiazol-2-yl)pyridin- | 1.78 | 394 |
| 2-yl]pyridin-2-amine | | |
| 5-({[(1S)-1-Cyclohexylethyl]amino}methyl)-N-[6-(1,3- | 1.82 | 394 |
| thiazol-2-yl)pyridin-2-yl]pyridin-2-amine | | |
| 5-{[(1-Methyl-2-pyrrolidin-1-ylethyl)amino]methyl}-N-[6- | 0.81 | 395 |
| (1,3-thiazol-2-yl)pyridin-2-yl]pyridin-2-amine | | |
| 5-({[2-(3,4-Dihydroisoquinolin-2(1H)-yl)-1- | 1.49 | 457 |
| methylethyl]amino}methyl)-N-[6-(1,3-thiazol-2-yl)pyridin-2- | | |
| yl]pyridin-2-amine | | |
| 5-({[1-Methyl-2-(4-methylpiperazin-1- | 0.77 | 424 |
| yl)ethyl]amino}methyl)-N-[6-(1,3-thiazol-2-yl)pyridin-2- | | |
| yl]pyridin-2-amine | | |
| 5-({[1-Methyl-2-(4-phenylpiperazin-1- | 1.82 | 486 |
| yl)ethyl]amino}methyl)-N-[6-(1,3-thiazol-2-yl)pyridin-2- | | |
| yl]pyridin-2-amine | | |
| 5-{[(1-Methyl-2-morpholin-4-ylethyl)amino]methyl}-N-[6- | 1.4 | 411 |
| (1,3-thiazol-2-yl)pyridin-2-yl]pyridin-2-amine | | |
| 2-{[(6-{[6-(1,3-Thiazol-2-yl)pyridin-2-yl]amino}pyridin-3- | 0.88 | 342 |
| yl)methyl]amino}propan-1-ol | | |
| N¹,N¹-Diethyl-N⁴-[(6-{[6-(1,3-thiazol-2-yl)pyridin-2- | 0.86 | 425 |
| yl]amino}pyridin-3-yl)methyl]pentane-1,4-diamine | | |
| 5-({[(1-Ethylpyrrolidin-2-yl)methyl]amino}methyl)-N-[6- | 0.73 | 395 |
| (1,3-thiazol-2-yl)pyridin-2-yl]pyridin-2-amine | | |
| 5-{[(2-Pyrrolidin-1-ylethyl)amino]methyl}-N-[6-(1,3-thiazol- | 0.75 | 381 |
| 2-yl)pyridin-2-yl]pyridin-2-amine | | |
| 5-({[2-(1,3-Dihydro-2H-isoindol-2-yl)ethyl]amino}methyl)- | 1.23 | 429 |
| N-[6-(1,3-thiazol-2-yl)pyridin-2-yl]pyridin-2-amine | | |
| 5-({[2-(1-Methylpyrrolidin-2-yl)ethyl]amino}methyl)-N-[6- | 0.63 | 395 |
| (1,3-thiazol-2-yl)pyridin-2-yl]pyridin-2-amine | | |
| 5-{[(2-Piperidin-1-ylethyl)amino]methyl}-N-[6-(1,3-thiazol- | 0.9 | 395 |
| 2-yl)pyridin-2-yl]pyridin-2-amine | | |
| N-Ethyl-N-(3-methylphenyl)-N′-[(6-{[6-(1,3-thiazol-2- | 1.85 | 445 |
| yl)pyridin-2-yl]amino}pyridin-3-yl)methyl]ethane-1,2- | | |
| diamine | | |
| 5-{[(2-Methoxyethyl)amino]methyl}-N-[6-(1,3-thiazol-2- | 1 | 342 |
| yl)pyridin-2-yl]pyridin-2-amine | | |
| N,N-Diethyl-N′-[(6-{[6-(1,3-thiazol-2-yl)pyridin-2- | 0.78 | 397 |
| yl]amino}pyridin-3-yl)methyl]propane-1,3-diamine | | |
| 5-{[(3-Pyrrolidin-1-ylpropyl)amino]methyl}-N-[6-(1,3- | 0.74 | 395 |
| thiazol-2-yl)pyridin-2-yl]pyridin-2-amine | | |
| 1-(3-{[(6-{[6-(1,3-Thiazol-2-yl)pyridin-2-yl]amino}pyridin-3- | 1.1 | 409 |
| yl)methyl]amino}propyl)pyrrolidin-2-one | | |
| 5-({[3-(4-Methylpiperazin-1-yl)propyl]amino}methyl)-N-[6- | 0.7 | 424 |
| (1,3-thiazol-2-yl)pyridin-2-yl]pyridin-2-amine | | |
| 5-{[(3-Morpholin-4-ylpropyl)amino]methyl}-N-[6-(1,3- | 0.7 | 411 |
| thiazol-2-yl)pyridin-2-yl]pyridin-2-amine | | |
| N,N-Dimethyl-N′-[(6-{[6-(1,3-thiazol-2-yl)pyridin-2- | 0.63 | 355 |
| yl]amino}pyridin-3-yl)methyl]ethane-1,2-diamine | | |
| 5-{[Benzyl(methyl)amino]methyl}-N-[6-(1,3-thiazol-2- | 1.49 | 388 |
| yl)pyridin-2-yl]pyridin-2-amine | | |
| {Methyl[(6-{[6-(1,3-thiazol-2-yl)pyridin-2-yl]amino}pyridin- | 1.47 | 337 |
| 3-yl)methyl]amino}acetonitrile | | |
| Ethyl {Methyl[(6-{[6-(1,3-thiazol-2-yl)pyridin-2- | 1.33 | 384 |
| yl]amino}pyridin-3-yl)methyl]amino}acetate | | |
| 5-{[Methyl(2-phenylethyl)amino]methyl}-N-[6-(1,3-thiazol- | 1.63 | 402 |
| 2-yl)pyridin-2-yl]pyridin-2-amine | | |
| N,N-Diethyl-N′-methyl-N′-[(6-{[6-(1,3-thiazol-2-yl)pyridin-2- | 1.08 | 397 |
| yl]amino}pyridin-3-yl)methyl]ethane-1,2-diamine | | |
| 2-{Methyl[(6-{[6-(1,3-thiazol-2-yl)pyridin-2- | 0.77 | 342 |
| yl]amino}pyridin-3-yl)methyl]amino}ethanol | | |
| 5-{[(1-Benzylazetidin-3-yl)(methyl)amino]methyl}-N-[6-(1,3- | 1.54 | 443 |
| thiazol-2-yl)pyridin-2-yl]pyridin-2-amine | | |
| 5-{[Cyclohexyl(methyl)amino]methyl}-N-[6-(1,3-thiazol-2- | 1.42 | 380 |
| yl)pyridin-2-yl]pyridin-2-amine | | |
| 5-({Methyl[(1R,2R)-2-pyrrolidin-1- | 1.46 | 449 |
| ylcyclohexyl]amino}methyl)-N-[6-(1,3-thiazol-2-yl)pyridin-2- | | |
| yl]pyridin-2-amine | | |
| 5-({Methyl[(1R,2R)-2-morpholin-4- | 1.52 | 465 |
| ylcyclohexyl]amino}methyl)-N-[6-(1,3-thiazol-2-yl)pyridin-2- | | |
| yl]pyridin-2-amine | | |
| N,N,N′-Trimethyl-N′-[(6-{[6-(1,3-thiazol-2-yl)pyridin-2- | 1.35 | 421 |
| yl]amino}pyridin-3-yl)methyl]cyclohex-4-ene-1,2-diamine | | |
| 5-{[Methyl(1-methylpiperidin-4-yl)amino]methyl}-N-[6-(1,3- | 0.6 | 395 |
| thiazol-2-yl)pyridin-2-yl]pyridin-2-amine | | |
| 5-[(Diethylamino)methyl]-N-[6-(1,3-thiazol-2-yl)pyridin-2- | 0.98 | 340 |
| yl]pyridin-2-amine | | |
| 5-{[Benzyl(ethyl)amino]methyl}-N-[6-(1,3-thiazol-2- | 1.55 | 402 |
| yl)pyridin-2-yl]pyridin-2-amine | | |
| N,N,N′-Triethyl-N′-[(6-{[6-(1,3-thiazol-2-yl)pyridin-2- | 1.22 | 411 |
| yl]amino}pyridin-3-yl)methyl]ethane-1,2-diamine | | |
| 2-{Ethyl[(6-{[6-(1,3-thiazol-2-yl)pyridin-2-yl]amino}pyridin- | 0.87 | 356 |
| 3-yl)methyl]amino}ethonal | | |
| 5-{[Cyclohexyl(ethyl)amino]methyl}-N-[6-(1,3-thiazol-2- | 1.48 | 394 |
| yl)pyridin-2-yl]pyridin-2-amine | | |
| 5-{[Benzyl(isopropyl)amino]methyl}-N-[6-(1,3-thiazol-2- | 1.56 | 416 |
| yl)pyridin-2-yl]pyridin-2-amine | | |
| 5-{[Isopropyl(2-methoxyethyl)amino]methyl}-N-[6-(1,3- | 1.17 | 384 |
| thiazol-2-yl)pyridin-2-yl]pyridin-2-amine | | |
| 5-{[Bis(2-methoxyethyl)amino]methyl}-N-[6-(1,3-thiazol-2- | 1.26 | 400 |
| yl)pyridin-2-yl]pyridin-2-amine | | |
| 5-[(Dibutylamino)methyl]-N-[6-(1,3-thiazol-2-yl)pyridin-2- | 1.73 | 396 |
| yl]pyridin-2-amine | | |
| 5-[(Dicyclohexylamino)methyl]-N-[6-(1,3-thiazol-2- | 1.85 | 448 |
| yl)pyridin-2-yl]pyridin-2-amine | | |
| Diethyl 2,2′-{[(6-{[6-(1,3-Thiazol-2-yl)pyridin-2- | 2.11 | 456 |
| yl]amino}pyridin-3-yl)methyl]imino}diacetate | | |
| 3-{Benzyl[(6-{[6-(1,3-thiazol-2-yl)pyridin-2- | 2.22 | 427 |
| yl]amino}pyridin-3-yl)methyl]amino}propanenitrile | | |
| 2-{Benzyl[(6-{[6-(1,3-thiazol-2-yl)pyridin-2- | 1.51 | 418 |
| yl]amino}pyridin-3-yl)methyl]amino}ethanol | | |
| 5-[(Diisobutylamino)methyl]-N-[6-(1,3-thiazol-2-yl)pyridin- | 1.7 | 396 |
| 2-yl]pyridin-2-amine | | |
| 5-[(Dipropylamino)methyl]-N-[6-(1,3-thiazol-2-yl)pyridin-2- | 1.34 | 368 |
| yl]pyridin-2-amine | | |
| 5-{[Ethyl(propyl)amino]methyl}-N-[6-(1,3-thiazol-2- | 1.16 | 354 |
| yl)pyridin-2-yl]pyridin-2-amine | | |
| 1-[(6-{[6-(1,3-Thiazol-2-yl)pyridin-2-yl]amino}pyridin-3- | 1.05 | 340 |
| yl)methyl]azetidin-3-ol | | |
| 5-[(3-Piperidin-1-ylazetidin-1-yl)methyl]-N-[6-(1,3-thiazol-2- | 1.22 | 407 |
| yl)pyridin-2-yl]pyridin-2-amine | | |
| 5-(Pyrrolidin-1-ylmethyl)-N-[6-(1,3-thiazol-2-yl)pyridin-2- | 0.95 | 338 |
| yl]pyridin-2-amine | | |
| 5-{[(2S)-2-(Methoxymethyl)pyrrolidin-1-yl]methyl}-N-[6- | 1.18 | 382 |
| (1,3-thiazol-2-yl)pyridin-2-yl]pyridin-2-amine | | |
| 5-(1,3-Dihydro-2H-isoindol-2-ylmethyl)-N-[6-(1,3-thiazol-2- | 1.36 | 386 |
| yl)pyridin-2-yl]pyridin-2-amine | | |
| 5-(Piperidin-1-ylmethyl)-N-[6-(1,3-thiazol-2-yl)pyridin-2- | 1.08 | 352 |
| yl]pyridin-2-amine | | |
| 1-[(6-{[6-(1,3-Thiazol-2-yl)pyridin-2-yl]amino}pyridin-3- | 1.22 | 368 |
| yl)methyl]piperidin-3-ol | | |
| 5-[(4-Methylpiperidin-1-yl)methyl]-N-[6-(1,3-thiazol-2- | 1.29 | 366 |
| yl)pyridin-2-yl]pyridin-2-amine | | |
| 1-[(6-{[6-(1,3-Thiazol-2-yl)pyridin-2-yl]amino}pyridin-3- | 0.81 | 395 |
| yl)methyl]piperidine-4-carboxamide | | |
| Ethyl 1-[(6-{[6-(1,3-Thiazol-2-yl)pyridin-2-yl]amino}pyridin- | 1.35 | 424 |
| 3-yl)methyl]piperidine-4-carboxylate | | |
| 1-[(6-{[6-(1,3-Thiazol-2-yl)pyridin-2-yl]amino}pyridin-3- | 0.83 | 368 |
| yl)methyl]piperidin-4-ol | | |
| 5-[(4-Benzylpiperidin-1-yl)methyl]-N-[6-(1,3-thiazol-2- | 1.95 | 442 |
| yl)pyridin-2-yl]pyridin-2-amine | | |
| 5-(1,4′-Bipiperidin-1′-ylmethyl)-N-[6-(1,3-thiazol-2- | 1.73 | 435 |
| yl)pyridin-2-yl]pyridin-2-amine | | |
| 5-[(2,6-Dimethylpiperidin-1-yl)methyl]-N-[6-(1,3-thiazol-2- | 1.25 | 380 |
| yl)pyridin-2-yl]pyridin-2-amine | | |
| 5-[(2,2,6,6-Tetramethylpiperidin-1-yl)methyl]-N-[6-(1,3- | 1.29 | 408 |
| thiazol-2-yl)pyridin-2-yl]pyridin-2-amine | | |
| 5-(3,6-Dihydropyridin-1(2H)-ylmethyl)-N-[6-(1,3-thiazol-2- | 1.02 | 350 |
| yl)pyridin-2-yl]pyridin-2-amine | | |
| 5-[(4-Phenyl-3,6-dihydropyridin-1(2H)-yl)methyl]-N-[6-(1,3- | 1.77 | 426 |
| thiazol-2-yl)pyridin-2-yl]pyridin-2-amine | | |
| 5-(3,4-Dihydroisoquinolin-2(1H)-ylmethyl)-N-[6-(1,3-thiazol- | 1.47 | 400 |
| 2-yl)pyridin-2-yl]pyridin-2-amine | | |
| 5-[(4-Methylpiperazin-1-yl)methyl]-N-[6-(1,3-thiazol-2- | 1.58 | 367 |
| yl)pyridin-2-yl]pyridin-2-amine | | |
| 5-[(4-Isopropylpiperazin-1-yl)methyl]-N-[6-(1,3-thiazol-2- | 1.12 | 395 |
| yl)pyridin-2-yl]pyridin-2-amine | | |
| Ethyl 4-[(6-{[6-(1,3-Thiazol-2-yl)pyridin-2-yl]amino}pyridin- | 1.31 | 425 |
| 3-yl)methyl]piperazine-1-carboxylate | | |
| 5-{[4-(2-Methoxyethyl)piperazin-1-yl]methyl}-N-[6-(1,3- | 1.14 | 411 |
| thiazol-2-yl)pyridin-2-yl]pyridin-2-amine | | |
| 5-[(4-Phenylpiperazin-1-yl)methyl]-N-[6-(1,3-thiazol-2- | 1.62 | 429 |
| yl)pyridin-2-yl]pyridin-2-amine | | |
| 5-{[4-(2-Methoxyphenyl)piperazin-1-yl]methyl}-N-[6-(1,3- | 1.69 | 459 |
| thiazol-2-yl)pyridin-2-yl]pyridin-2-amine | | |
| 5-{[4-(3-Methoxyphenyl)piperazin-1-yl]methyl}-N-[6-(1,3- | 1.78 | 459 |
| thiazol-2-yl)pyridin-2-yl]pyridin-2-amine | | |
| 5-[(4-Benzylpiperazin-1-yl)methyl]-N-[6-(1,3-thiazol-2- | 1.47 | 443 |
| yl)pyridin-2-yl]pyridin-2-amine | | |
| 5-(Morpholin-4-ylmethyl)-N-[6-(1,3-thiazol-2-yl)pyridin-2- | 0.87 | 354 |
| yl]pyridin-2-amine | | |
| 5-[(2,6-Dimethylmorpholin-4-yl)methyl]-N-[6-(1,3-thiazol-2- | 1.22 | 382 |
| yl)pyridin-2-yl]pyridin-2-amine | | |
| N-[6-(1,3-Thiazol-2-yl)pyridin-2-yl]-5-(thiomorpholin-4- | 1.07 | 370 |
| ylmethyl)pyridin-2-amine | | |
| 5-(Azepan-1-ylmethyl)-N-[6-(1,3-thiazol-2-yl)pyridin-2- | 1.23 | 366 |
| yl]pyridin-2-amine | | |
| 5-[(4-Methyl-1,4-diazepan-1-yl)methyl]-N-[6-(1,3-thiazol-2- | 1.59 | 381 |
| yl)pyridin-2-yl]pyridin-2-amine | | |
| 5-[(4-Propylpiperazin-1-yl)methyl]-N-[6-(1,3-thiazol-2- | 1.17 | 395 |
| yl)pyridin-2-yl]pyridin-2-amine | | |
| 5-{[4-(2-Fluorobenzoyl)piperazin-1-yl]methyl}-N-[6-(1,3- | 1.48 | 475 |
| thiazol-2-yl)pyridin-2-yl]pyridin-2-amine | | |
| 5-{[4-(3-Fluorobenzoyl)piperazin-1-yl]methyl}-N-[6-(1,3- | 1.52 | 475 |
| thiazol-2-yl)pyridin-2-yl]pyridin-2-amine | | |
| 5-{[4-(4-Fluorobenzoyl)piperazin-1-yl]methyl}-N-[6-(1,3- | 1.52 | 475 |
| thiazol-2-yl)pyridin-2-yl]pyridin-2-amine | | |
| 5-{[4-(3-Methoxypropyl)piperazin-1-yl]methyl}-N-[6-(1,3- | 1.18 | 425 |
| thiazol-2-yl)pyridin-2-yl]pyridin-2-amine | | |
| 5-{[4-(2-Methoxybenzyl)piperazin-1-yl]methyl}-N-[6-(1,3- | 1.56 | 473 |
| thiazol-2-yl)pyridin-2-yl]pyridin-2-amine | | |
| 5-{[4-(3-Methoxybenzyl)piperazin-1-yl]methyl}-N-[6-(1,3- | 1.57 | 473 |
| thiazol-2-yl)pyridin-2-yl]pyridin-2-amine | | |
| 5-{[4-(4-Methoxybenzyl)piperazin-1-yl]methyl}-N-[6-(1,3- | 1.56 | 473 |
| thiazol-2-yl)pyridin-2-yl]pyridin-2-amine | | |
| 5-{[4-(Pyridin-4-ylmethyl)piperazin-1-yl]methyl}-N-[6-(1,3- | 0.75 | 444 |
| thiazol-2-yl)pyridin-2-yl]pyridin-2-amine | | |
| {1-[(6-{[6-(1,3-Thiazol-2-yl)pyridin-2-yl]amino}pyridin-3- | 0.91 | 382 |
| yl)methyl]piperidin-4-yl}methanol | | |
| 5-(Anilinomethyl)-N-[6-(1,3-thiazol-2-yl)pyridin-2- | 2.03 | 360 |
| yl]pyridin-2-amine | | |
| 5-{[(2-Methoxyphenyl)amino]methyl}-N-[6-(1,3-thiazol-2- | 2.08 | 390 |
| yl)pyridin-2-yl]pyridin-2-amine | | |
| 5-[(1,3-Thiazol-2-ylamino)methyl]-N-[6-(1,3-thiazol-2- | 1.21 | 367 |
| yl)pyridin-2-yl]pyridin-2-amine | | |
| 5-[(Pyridin-2-ylamino)methyl]-N-[6-(1,3-thiazol-2-yl)pyridin- | 1.08 | 361 |
| 2-yl]pyridin-2-amine | | |
| 5-[(Pyridin-3-ylamino)methyl]-N-[6-(1,3-thiazol-2-yl)pyridin- | 0.91 | 361 |
| 2-yl]pyridin-2-amine | | |
| 2-{4-[(6-{[6-(1,3-Thiazol-2-yl)pyridin-2-yl]amino}pyridin-3- | 1.04 | 397 |
| yl)methyl]piperazin-1-yl}ethanol | | |
| 5-[(Pyridin-4-ylamino)methyl]-N-[6-(1,3-thiazol-2-yl)pyridin- | 0.9 | 361 |
| 2-yl]pyridin-2-amine | | |
| N-[(6-{[6-(1,3-Thiazol-2-yl)pyridin-2-yl]amino}pyridin-3- | 1.59 | 362 |
| yl)methyl]pyrazin-2-amine | | |
| N-[(6-{[6-(1,3-Thiazol-2-yl)pyridin-2-yl]amino}pyridin-3- | 1.53 | 362 |
| yl)methyl]pyrimidin-2-amine | | |

### Example 22

### Preparation of 27

22.1 Synthesis

A solution of 0.57 mmol of 18 in 5 mL of formic acid was stirred at 100° C. for 10 h. After the solvents were removed, the residue was dissolved in 10 mL of chloroform and the organic solution was washed with saturated NaHCO₃, saturated NaCl, dried over MgSO₄, and concentrated under a reduced pressure. The crude product was purified by column chromatography on silica gel to give 0.29 mmol of 27.

22.2 Results

Analytical data for exemplary compound of structure 27 is provided below.

22.2.a 3,5-Dipyridin-2-yl-3H-imidazo[4,5-b]pyridine

¹H NMR (400 MHz, CDCl₃) δ 9.24 (s, 1H), 8.98 (d, J=8.8 Hz, 1H), 8.72 (d, J=4.0 Hz, 1H), 8.53-7.55 (m, 2H), 8.49 (d, J=7.6 Hz, 1H), 8.24 (d, J=8.8 Hz, 1H), 8.00 (t, J=7.6 Hz, 1H), 7.86 (t, J=7.6 Hz, 1H), 7.31-7.34 (m, 2H); MS m/z: 372 (M+1).

### Example 23

### Preparation of the Metal Complex 28

23.1 Synthesis

To a solution of 0.2 mmol of 18 in EtOH at 60° C. was added 0.1 mL of 1.0 M FeClO₄ in ether and a white precipitate forms immediately. To the mixture was added 0.06 mL of triethyl amine and the resulting mixture was stirred for 20 min. After the mixture was cooled to rt, the white precipitate was filtered to yield 60% of 28.

23.2 Results

Analytical data for exemplary compounds of structure 28 are provided below.

23.2.a {[2,2′]Bipyridinyl-6-yl-pyridin-2-yl-amine}₂Fe (II) Complex

MS m/z: 551 (M+1).

### Examples 23A-23F

### Example 23A

### 6-Iodo-3-methoxy-N-pyridin-2-ylpyridin-2-amine

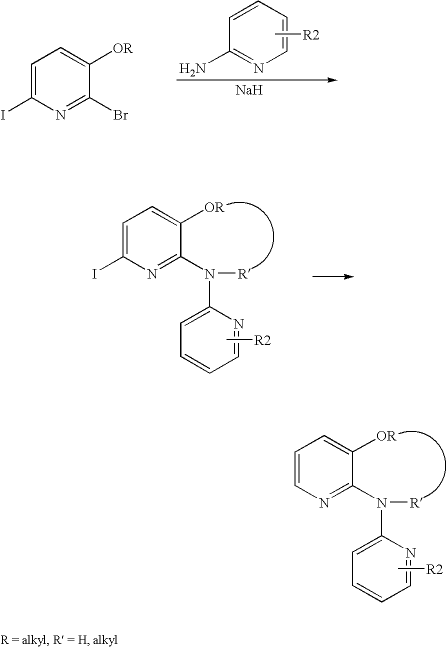

To a solution of 1.68 mmol of 2-bromo-6-iodo-3-methoxypyridine in 10 mL of anhydrous THF was added 2.52 mmol of NaH (60% in mineral oil) followed by the addition of 1.85 mmol of 2-aminopyridine, and the resulting mixture was stirred at 50° C. for 8 h. After the reaction was quenched with methanol, the solvents were removed. The residue was dissolved in 20 mL of ethyl acetate and the organic solution was washed with saturated NaCl, dried over MgSO₄, and concentrated under a reduced pressure. The crude product was purified by column chromatography on silica gel to give 0.97 mmol of 6-iodo-3-methoxy-N-pyridin-2-ylpyridin-2-amine.

¹H NMR (400 MHz, CDCl₃) δ 8.40 (d, J=8.0 Hz, 1H), 8.24 (d, J=4.8 Hz, 1H), 7.70 (t, J=8.0 Hz, 1H), 7.83 (br s, 1H), 7.14 (d, J=8.0 Hz, 1H), 6.90 (dd, J=4.8, 8.0 Hz, 1H), 6.67 (d, J=8.0 Hz, 1H), 3.86 (s, 3H); MS m/z: 328 (M+1).

### 6-Iodo-3-isopropoxy-N-pyridin-2-ylpyridin-2-amine

¹H NMR (400 MHz, CDCl₃) δ 8.42 (d, J=8.4 Hz, 1H), 8.24-8.26 (m, 1H), 7.70 (t, J=7.2 Hz, 1H), 7.82 (br s, 1H), 7.13 (d, J=8.4 Hz, 1H), 6.90 (dd, J=4.8, 7.2 Hz, 1H), 6.68 (d, J=8.4 Hz, 1H), 4.55-4.59 (m, 1H), 1.39 (d, J=6.0 Hz, 6H); MS m/z: 356 (M+1).

### 3-(Benzyloxy)-6-iodo-N-pyridin-2-ylpyridin-2-amine

¹H NMR (400 MHz, CDCl₃) δ 8.41 (d, J=8.0 Hz, 1H) 8.21 (d, J=4.8 Hz, 1H), 7.85 (br s, 1H), 7.68 (t, J=8.0 Hz, 1H), 7.35-7.41 (m, 5H), 7.09 (d, J=8.0 Hz, 1H), 6.89 (dd, J=4.8, 8.0 Hz, 1H), 6.70 (d, J=8.0 Hz, 1H), 5.07 (s, 2H); MS m/z: 404 (M+1).

### 6-Iodo-3-(2-methoxyethoxy)-N-pyridin-2-ylpyridin-2-amine

¹H NMR (400 MHz, CDCl₃) δ 8.41 (d, J=8.0 Hz, 1H), 8.24 (d, J=5.2 Hz, 1H), 7.72 (t, J=8.0 Hz, 1H), 7.90 (br s, 1H), 7.14 (d, J=8.0 Hz, H), 6.91 (dd, J=5.2, 8.0 Hz, 1H), 6.74 (d, J=8.0 Hz, 1H), 4.16 (t, J=4.8 Hz, 2H), 3.77 (t, J=4.8 Hz, 2H), 3.44 (s, 3H); MS m/z: 372 (M+1).

### Methyl {[6-iodo-2-(pyridin-2-ylamino)pyridin-3-yl]oxy}acetate

¹H NMR (400 MHz, CDCl₃) δ 9.11 (br s, 1H) 8.33 (d, J=4.8 Hz, 1H), 8.23 (d, J=7.6 Hz, 1H), 7.72 (t, J=7.6 Hz, 1H), 7.25 (d, J=7.6 Hz, 1H), 7.08 (dd, J=4.8, 7.6 Hz, 1H), 6.86 (d, J=7.6 Hz, 1H), 4.61 (s, 2H), 4.03 (s, 3H); MS m/z: 386 (M+1).

### 6-Iodo-4-pyridin-2-yl-3,4-dihydro-2H-pyrido[3,2-b][1,4]oxazine

A mixture of 1.41 mmol of 4,6-dipyridin-2-yl-3,4-dihydro-2H-pyrido[3,2-b][1,4]oxazine, 1.41 mmol of 2-(tributylstannyl)pyridine, and 0.07 mmol of Pd(PPh₃)₄ in 10 mL of toluene was stirred at 100° C. for 15 h under Ar. The reaction was quenched with 10 mL of saturated NaHCO₃. After the mixture was extracted with chloroform, the organic phase was washed with saturated NaCl, dried over MgSO₄, and concentrated under reduced pressure. The residue was purified by column chromatography to give 1.16 mmol of 6-iodo-4-pyridin-2-yl-3,4-dihydro-2H-pyrido[3,2-b][1,4]oxazine.

MS m/z: 340 (M+1).

### Example 23B

### N⁶-Pyridin-2-yl-2,2′-bipyridine-5,6-diamine dihydrochloride

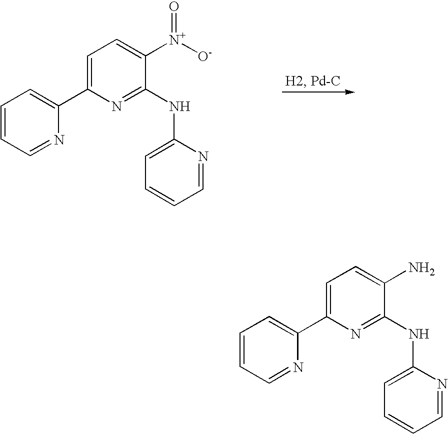

A suspension of 4.77 mmol of (5-nitro-2,2′-bipyridin-6-yl)(pyridin-2-yl)amine and 0.25 g of Pd/C (10%) in 100 mL of ethyl acetate and 7 mL of 4N HCl in ethyl acetate was stirred for 4 h under H₂ (1 atm). After filtering through celite, the solution was concentrated under a reduced pressure to give 4.57 mmol of N⁶-pyridin-2-yl-2,2′-bipyridine-5,6-diamine dihydrochloride

¹H NMR (400 MHz, DMSO-d₆) δ 12.03 (br s, 1H), 8.80-8.85 (m, 2H), 8.03-8.45 (m, 6H), 7.63-7.69 (m, 2H), 7.29-7.39 (m, 2H); MS m/z: 264 (M+1).

### Example 23C

### N-[6-(pyridin-2-ylamino)-2,2′-bipyridin-5-yl]acetamide

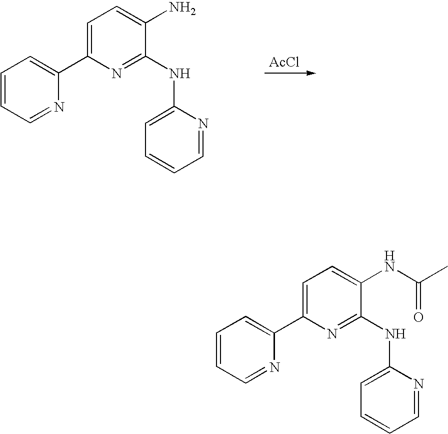

A mixture of 0.38 mmol of N⁶-pyridin-2-yl-2,2′-bipyridine-5,6-diamine, 0.57 mmol of acetyl chloride, and 1.14 mmol of triethylamine in 5 mL of chloroform was stirred at 0° C. for 4 h. After the reaction was quenched with saturated NaHCO₃, the mixture was diluted with chloroform. The organic solution was washed with saturated NaCl, dried over MgSO₄, and concentrated under reduced pressure. The crude product was purified by column chromatography on silica gel to give 0.23 mmol of N-[6-(pyridin-2-ylamino)-2,2′-bipyridin-5-yl]acetamide.

¹H NMR (400 MHz, CDCl₃) δ 8.68-8.69 (m, 1H), 8.51 (d, J=8.0 Hz, 1H), 8.47 (d, J=8.0 Hz, 1H), 8.35 (d, J=8.0 Hz, 1H), 8.23-8.27 (m, 1H), 7.65-7.80 (m, 4H), 7.30-7.35 (m, 1H), 7.05-7.09 (m, 1H), 2.17 (s, 3H); MS m/z: 306 (M+1).

### Example 23D

### 6-(Pyridin-2-ylamino)-2,2′-bipyridin-5-ol

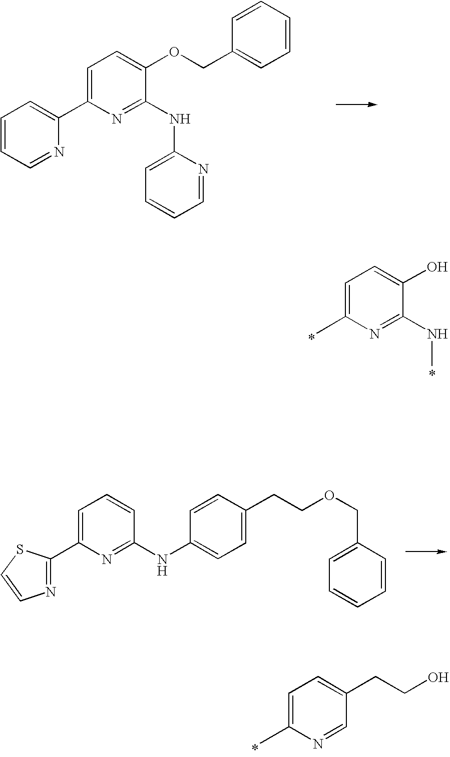

A suspension of 0.45 mmol of 5-(benzyloxy)-N-pyridin-2-yl-2,2′-bipyridin-6-amine and 48 mg of Pd/C (10%) in 10 mL of ethanol was stirred for 5 h under H₂ (1 atm). After filtering through celite, the solution was concentrated under a reduced pressure. The crude product was purified by column chromatography on silica gel to give 0.38 mmol of 6-(pyridin-2-ylamino)-2,2′-bipyridin-5-ol.

¹H NMR (400 MHz, CDCl₃) δ 8.63 (d, J=4.0 Hz, 1H), 8.15-8.25 (m, 3H), 7.63-7.76 (m, 4H), 7.52 (br s, 1H), 7.15-7.19 (m, 2H), 6.97 (br s, 1H); MS m/z: 265 (M+1).

### 2-(6-{[6-(1,3-Thiazol-2-yl)pyridin-2-yl]amino}pyridin-3-yl)ethanol dihydrochloride

¹H NMR (400 MHz, DMSO-d₆) δ 12.71 (s, 1H), 8.38 (br s, 1H), 8.26 (dd, J=2.0, 8.8 Hz, 1H), 8.05-8.09 (m, 2H), 8.02 (d, J=3.6 Hz, 1H), 7.90 (d, J=7.6 Hz, 1H), 7.84 (d, J=8.8 Hz, 1H), 7.43 (d, J=7.6 Hz, 1H), 3.68 (t, J=6.4 Hz, 2H), 2.81 (t, J=6.4 Hz, 2H); MS m/z: 299 (M+1).

### Example 23E

### 5-(1-Methylpiperidin-3-yl)pyridin-2-amine

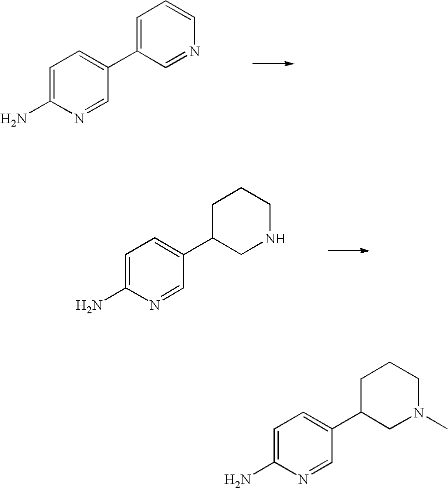

A solution of 1.2. mmol of 3,3′-bipyridin-6-amine in 40 mL of AcOH was hydrogenated with catalytic amount of PtO₂ at 3-4 atom hydrogen atmosphere at room temperature for 5 days. The reaction mixture was filtered through celite and concentrated in vacuo. The residue was diluted with saturated NaHCO₃ and extracted with CHCl₃. The organic phase was washed with brine, dried over MgSO₄, and concentrated. The residue was roughly purified by column chromatography on silica gel to give a crude 5-piperidin-3-ylpyridin-2-amine (8.2 mmol).

To a solution of the product in 20 mL of MeOH was added 8.2 mmol of iodomethane at room temperature and stirred for 30 min. The reaction mixture was neutralized with aqueous K₂CO₃ and concentrated in vacuo. The residue was diluted with CHCl₃-MeOH (10:1) solution and filtered through celite. The filtrate was concentrated and the residue was purified by column chromatography on silica gel to give 1.98 mmol of 5-(1-methylpiperidin-3-yl)pyridin-2-amine.

¹H NMR (300 MHz, CDCl₃) δ 7.93 (d, J=2.2 Hz, 1H), 7.31 (dd, J=2.4 Hz, 8.4 Hz, 1H), 6.45 (d, J=8.4 Hz, 1H), 4.37 (br s, 2H), 2.83-2.92 (m, 2H), 2.64-2.77 (m, 1H), 2.29 (s, 3H), 1.65-1.94 (m, 5H), 1.20-1.45 (m, 1H); MS m/z: 192 (M+1).

### Example 23F

### 1-(6-{[6-(1,3-Thiazol-2-yl)pyridin-2-yl]amino}pyridin-3-yl)piperazin-2-one dihydrochloride

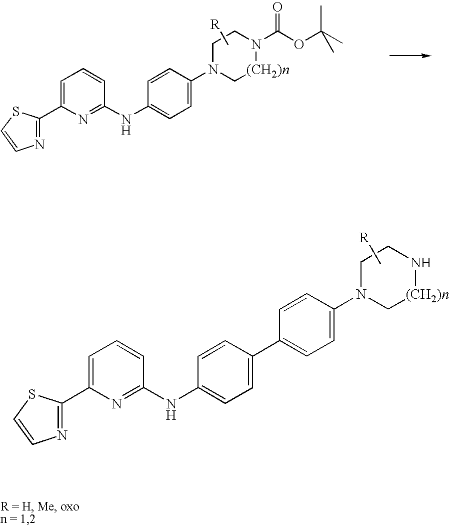

A solution of tert-butyl 3-oxo-4-(6-{[6-(1,3-thiazol-2-yl)pyridin-2-yl]amino}pyridin-3-yl)piperazine-1-carboxylate in 4N HCl-dioxane was stirred at 60° C. for 1 h. After cooling to room temperature, the mixture was concentrated in vacuo and recrystallizatin from aqueous ethanol to give 0.20 mmol of 1-(6-{[6-(1,3-thiazol-2-yl)pyridin-2-yl]amino}pyridin-3-yl)piperazin-2-one dihydrochloride

¹H NMR (400 MHz, DMSO-d₆) δ 10.52-10.64 (br, 1H), 10.00-10.12 (br, 2H), 8.32 (d, J=2.5 Hz, 1H), 8.07 (d, J=8.8 Hz, 1H), 8.01 (d, J=2.9 Hz1, H), 7.90 (d, J=2.9 Hz, 1H), 7.89 (dd, J=7.3, 8.3 Hz, 1H), 7.84 (dd, J=2.5, 8.8 Hz, 1H), 7.71 (d, J=7.3 Hz, 1H), 7.59 (d, J=8.3 Hz, 1H), 3.90-4.00 (m, 4H), 3.50-3.60 (m, 2H); MS m/z: 353 (M+1).

### 1-(6-{[3-Methoxy-6-(1,3-thiazol-2-yl)pyridin-2-yl]amino}pyridin-3-yl)piperazin-2-one dihydrochloride

¹H NMR (400 MHz, DMSO-d₆) δ 9.88-10.00 (br, 2H), 8.90-9.00 (br, 1H), 8.48 (d, J=8.8 Hz, 1H), 8.37 (d, J=2.5 Hz, 1H), 7.97 (dd, J=2.5, 8.8 Hz, 1H), 7.94 (d, J=3.0 Hz, 1H), 7.81 (d, J=3.0 Hz, 1H), 7.77 (d, J=8.3 Hz, 1H), 7.55 (d, J=8.3 Hz, 1H), 4.01 (s, 3H), 3.88-3.99 (m, 4H), 3.60-3.80 (m, 2H); MS m/z: 383 (M+1).

### 3-Methoxy-N-(5-piperazin-1-ylpyridin-2-yl)-6-(1,3-thiazol-2-yl)pyridin-2-amine dihydrochloride

¹H NMR (400 MHz, DMSO-d₆) δ 10.22 (br s, 1H), 9.68 (br s, 2H), 8.34 (d, J=9.3 Hz, 1H), 8.18 (dd, J=2.9, 9.8 Hz, 1H), 8.12 (d, J=2.9 Hz, 1H), 7.98 (d, J=2.9 Hz, 1H), 7.86 (d, J=2.9 Hz, 1H), 7.81 (d, J=8.3 Hz, 1H), 7.61 (d, J=8.3 Hz, 1H), 4.01 (s, 3H), 3.51-3.54 (m, 4H), 3.24 (m, 4H); MS m/z: 369 (M+1).

### 3-Methoxy-N-[5-(3-methylpiperazin-1-yl)pyridin-2-yl]-6-(1,3-thiazol-2-yl)pyridin-2-amine dihydrochloride

¹H NMR (400 MHz, DMSO-d₆) δ 10.30 (br s, 1H), 9.78 (br s, 1H), 9.66-9.68 (m, 1H), 8.34 (d, J=9.3 Hz, 1H), 8.22 (dd, J=2.4, 9.8 Hz, 1H), 8.14 (d, J=2.9 Hz, 1H), 7.97 (d, J=3.4 Hz, 1H), 7.87 (d, J=2.9 Hz, 1H), 7.83 (d, J=8.3 Hz, 1H), 7.63 (d, J=8.3 Hz, 1H), 4.02 (s, 3H), 3.82-3.90 (m, 2H), 3.37-3.40 (m, 2H), 3.10-3.24 (m, 2H), 2.98 (dd, J=12.7, 12.7 Hz, 1H), 1.35 (d, J=6.3 Hz, 6H); MS m/z: 383 (M+1).

### N-[5-(3,5-Dimethylpiperazin-1-yl)pyridin-2-yl]-3-methoxy-6-(1,3-thiazol-2-yl)pyridin-2-amine dihydrochloride

¹H NMR (400 MHz, DMSO-d₆) δ 10.21 (br s1, H), 9.95 (br s, 1H), 9.53-9.56 (m, 1H), 8.36 (d, J=9.8 Hz, 1H), 8.21 (dd, J=2.9, 9.8 Hz, 1H), 8.15 (d, J=2.9 Hz, 1H), 7.96 (d, J=3.5 Hz, 1H), 7.86 (d, J=2.9 Hz, 1H), 7.81 (d, J=8.3 Hz, 1H), 7.60 (d, J=8.3 Hz, 1H), 4.01 (s, 3H), 3.93 (d, J=11.2 Hz, 2H), 3.36-3.40 (m, 2H), 2.94 (dd, J=12.7, 12.7 Hz, 2H), 1.36 (d, J=6.4 Hz, 3H); MS m/z: 397 (M+1).

### 4-(6-{[3-Methoxy-6-(1,3-thiazol-2-yl)pyridin-2-yl]amino}pyridin-3-yl)-1,4-diazepan-5-one dihydrochloride

¹HNMR (400 MHz, DMSO-d₆) δ9.60 (br s, 2H), 9.13 (br s, 1H), 8.45 (d, J=9.3 Hz, 1H), 8.37 (d, J=2.5 Hz, 1H), 8.00 (dd, J=2.5, 9.3 Hz1, H), 7.95 (d, J=2.9 Hz, 1H), 7.83 (d, J=2.9 Hz, 1H), 7.78 (d, J=7.8 Hz, 1H), 7.56 (d, J=8.3 Hz, 1H), 4.09-4.11 (m, 2H), 4.01 (s, 3H), 3.39-3.43 (m, 4H), 3.00-3.02 (m, 2H); MS m/z: 397 (M+1).

### 4-(6-{[6-(1,3-Thiazol-2-yl)pyridin-2-yl]amino}pyridin-3-yl)-1,4-diazepan-5-one dihydrochloride

¹H NMR (400 MHz, DMSO-d₆) δ 10.80 (br s, 1H), 9.72 (br s, 2H), 8.33 (d, J=1.9 Hz, 1H), 8.02-8.06 (m, 2H), 7.86-7.93 (m, 3H), 7.72 (d, J=7.4 Hz, 1H), 7.54 (d, J=8.3 Hz, 1H), 4.09-4.11 (m, 2H), 3.38-3.42 (m, 4H), 3.00-3.03 (m, 2H); MS m/z: 367 (M+1).

### Example 24

24.1 Assay for Compound Activity Towards hSK Channels

Cells expressing small conductance, calcium activated potassium channels, such as SK-like channels were loaded with ⁸⁶Rb⁺ by culture in media containing ⁸⁶RbCl. Following loading, the culture media was removed and the cells were washed in EBSS to remove residual traces of ⁸⁶Rb⁺. Cells were preincubated with the drug (0.01 to 30 μM in EBSS) and then ⁸⁶Rb⁺ efflux was stimulated by exposing cells to EBSS solution supplemented with a calcium ionophore, such as ionomycin, in the continued presence of the drug. After a suitable efflux period, the EBSS/ionophore solution was removed from the cells and the ⁸⁶Rb⁺ content was determined by Cherenkov counting (Wallac Trilux). Cells were then lysed with a SDS solution and the ⁸⁶Rb⁺ content of the lysate was determined. Percent ⁸⁶Rb⁺ efflux was calculated according to the following equation:

(⁸⁶Rb⁺ content in EBSS/(86Rb⁺ content in EBSS+⁸⁶Rb⁺ content of the lysate))×100

24.2 Results

Compounds tested in this assay, along with their hSK2 inhibitory activity, are provided in Table 3.

TABLE 3

| | hSK2 |
|---|---|
| | Inhibitory |
| Compound Name | Activity |
| (5-Methyl-pyridin-2-yl)-(6-thiazol-2-yl-pyridin-2-yl)-amine | +++ |
| (5-Fluoro-pyridin-2-yl)-[6-(4-methyl-pyrazol-1-yl)-pyridin-2-yl]-amine | +++ |
| (5-Fluoro-pyridin-2-yl)-[6-(4-methyl-pyrazol-1-yl)-pyridin-2-yl]-amine | +++ |
| (5-Isopropenyl-pyridin-2-yl)-[6-(4-methyl-pyrazol-1-yl)-pyridin-2-yl]-amine | +++ |
| (5-Methoxy-pyridin-2-yl)-[6-(4-methyl-pyrazol-1-yl)-pyridin-2-yl]-amine | +++ |
| (5-Furan-2-yl-pyridin-2-yl)-(6-thiazol-2-yl-pyridin-2-yl)-amine | +++ |
| (5-Bromo-pyridin-2-yl)-(6-thiazol-2-yl-pyridin-2-yl)-amine | +++ |
| (5,6,7,8-Tetrahydro-isoquinolin-3-yl)-(6-thiazol-2-yl-pyridin-2-yl)-amine | +++ |
| (3-Methoxy-6-thiazol-2-yl-pyridin-2-yl)-(5-morpholin-4-yl-pyridin-2-yl)-amine | +++ |
| (5-Ethyl-pyridin-2-yl)-(6-thiazol-2-yl-pyridin-2-yl)-amine | +++ |
| [6-(5-Chloro-thiazol-2-yl)-pyridin-2-yl]-pyridin-2-yl-amine | +++ |
| (3-Methoxy-6-thiazol-2-yl-pyridin-2-yl)-(5-pyrrolidin-1-yl-pyridin-2-yl)-amine | +++ |
| 1-[6-(3-Methoxy-6-thiazol-2-yl-pyridin-2-ylamino)-pyridin-3-yl]-pyrrolidin-2-one | +++ |
| [6-(5-Methyl-thiazol-2-yl)-pyridin-2-yl]-pyridin-2-yl-amine | +++ |
| [6-(5-Chloro-thiazol-2-yl)-pyridin-2-yl]-(5-pyrrolidin-1-yl-pyridin-2-yl)-amine | +++ |
| 1-{6-[6-(5-Chloro-thiazol-2-yl)-pyridin-2-ylamino]-pyridin-3-yl}-pyrrolidin-2-one | +++ |
| N²-[6-(5-Chloro-thiazol-2-yl)-3-methoxy-pyridin-2-yl]-N⁵-(2-methoxy-ethyl)-N⁵- | +++ |
| methyl-pyridine-2,5-diamine | |
| [5-(3,4-Dihydro-1H-isoquinolin-2-ylmethyl)-pyridin-2-yl]-(6-thiazol-2-yl-pyridin- | +++ |
| 2-yl)-amine | |
| (6-Thiazol-2-yl-pyridin-2-yl)-(5-thiophen-3-yl-pyridin-2-yl)-amine | +++ |
| [5-(5-Methyl-furan-2-yl)-pyridin-2-yl]-(6-thiazol-2-yl-pyridin-2-yl)-amine | +++ |
| (5-Bromo-pyridin-2-yl)-(6-pyrazol-1-yl-pyridin-2-yl)-amine | +++ |
| (5-Bromo-pyridin-2-yl)-[6-(4-methyl-pyrazol-1-yl)-pyridin-2-yl]-amine | +++ |
| (5-Chloro-pyridin-2-yl)-(6-thiazol-2-yl-pyridin-2-yl)-amine | +++ |
| (5-Chloro-pyridin-2-yl)-(6-pyrazol-1-yl-pyridin-2-yl)-amine | +++ |
| (5-Isopropyl-pyridin-2-yl)-[6-(4-methyl-pyrazol-1-yl)-pyridin-2-yl]-amine | +++ |
| [5-(3-Fluoro-phenyl)-pyridin-2-yl]-[6-(4-methyl-pyrazol-1-yl)-pyridin-2-yl]-amine | +++ |
| [5-(2-Methoxy-phenyl)-pyridin-2-yl]-[6-(4-methyl-pyrazol-1-yl)-pyridin-2-yl]- | +++ |
| amine | |
| (5-Phenyl-2H-pyrazol-3-yl)-(6-thiazol-2-yl-pyridin-2-yl)-amine | +++ |
| [3,3′]Bipyridinyl-6-yl-(6-pyrazin-2-yl-pyridin-2-yl)-amine | +++ |
| (5-Furan-2-yl-pyridin-2-yl)-(6-pyrazin-2-yl-pyridin-2-yl)-amine | +++ |
| Isoquinolin-3-yl-(6-thiazol-2-yl-pyridin-2-yl)-amine | +++ |
| (3-Methoxy-6-thiazol-2-yl-pyridin-2-yl)-[5-(4-methyl-piperazin-1-yl)-pyridin-2- | +++ |
| yl]-amine | |
| (3-Methoxy-6-pyrazin-2-yl-pyridin-2-yl)-[5-(4-methyl-piperazin-1-yl)-pyridin-2- | +++ |
| yl]-amine | |
| (5-Methoxy-[2,2′]bipyridinyl-6-yl)-[5-(4-methyl-piperazin-1-yl)-pyridin-2-yl]- | +++ |
| amine | |
| [2,3′]Bipyridinyl-6′-yl-(3-methoxy-6-thiazol-2-yl-pyridin-2-yl)-amine | +++ |
| 3-[6-(6-Thiazol-2-yl-pyridin-2-ylamino)-pyridin-3-yl]-propionic acid ethyl ester | +++ |
| (3-Methoxy-6-thiazol-2-yl-pyridin-2-yl)-(3,4,5,6-tetrahydro-2H[1,3′]bipyridinyl- | +++ |
| 6′-yl)-amine | |
| (3-Methoxy-6-pyrazin-2-yl-pyridin-2-yl)-(3,4,5,6-tetrahydro-2H-[1,3′]bipyridinyl- | +++ |
| 6′-yl)-amine | |
| (3-Methoxy-6-thiazol-2-yl-pyridin-2-yl)-pyridin-2-yl-amine | +++ |
| (5-Methoxy-[2,2′]bipyridinyl-6-yl)-(3,4,5,6-tetrahydro-2H-[1,3′]bipyridinyl-6′-yl)- | +++ |
| amine | |
| (5-Isopropyl-pyridin-2-yl)-(3-methoxy-6-thiazol-2-yl-pyridin-2-yl)-amine | +++ |
| (5-Isopropyl-pyridin-2-yl)-(5-methoxy-[2,2′]bipyridinyl-6-yl)-amine | +++ |
| (5-Pyrrolidin-1-ylmethyl-pyridin-2-yl)-(6-thiazol-2-yl-pyridin-2-yl)-amine | +++ |
| [6-(5-Isopropyl-thiazol-2-yl)-pyridin-2-yl]-pyridin-2-yl-amine | +++ |
| [6-(5-Chloro-thiazol-2-yl)-3-methoxy-pyridin-2-yl]-pyridin-2-yl-amine | +++ |
| [6-(5-Ethyl-thiazol-2-yl)-pyridin-2-yl]-pyridin-2-yl-amine | +++ |
| 6′-(6-Pyrazol-1-yl-pyridin-2-ylamino)-3,4,5,6-tetrahydro-[1,3′]bipyridinyl-2-one | +++ |
| [6-(5-Chloro-thiazol-2-yl)-pyridin-2-yl]-(5-morpholin-4-yl-pyridin-2-yl)-amine | +++ |
| N²-[6-(5-Chloro-thiazol-2-yl)-pyridin-2-yl]-N⁵-(2-methoxy-ethyl)-N⁵-methyl- | +++ |
| pyridine-2,5-diamine | |
| [6-(5-Chloro-thiazol-2-yl)-3-methoxy-pyridin-2-yl]-(4-methoxy-3,4,5,6- | +++ |
| tetrahydro-2H-[1,3′]bipyridinyl-6′-yl)-amine | |
| [6-(5-Chloro-thiazol-2-yl)-3-methoxy-pyridin-2-yl]-(5-morpholin-4-yl-pyridin-2- | +++ |
| yl)-amine | |
| [6-(5-Chloro-thiazol-2-yl)-3-methoxy-pyridin-2-yl]-(5-pyrrolidin-1-yl-pyridin-2- | +++ |
| yl)-amine | |
| 1-{6-[6-(5-Chloro-thiazol-2-yl)-3-methoxy-pyridin-2-ylamino]-pyridin-3-yl}- | +++ |
| pyrrolidin-2-one | |
| (3-Methoxy-6-thiazol-2-yl-pyridin-2-yl)-(3,4,5,6-tetrahydro-2H-[1,3′]bipyridinyl- | +++ |
| 6′-yl)-amine | |
| (5-Chloro-pyridin-2-yl)-(6-pyrazin-2-yl-pyridin-2-yl)-amine | ++ |
| (5-Phenyl-pyridin-2-yl)-(6-thiazol-2-yl-pyridin-2-yl)-amine | ++ |
| [6-(4-Methyl-pyrazol-1-yl)-pyridin-2-yl]-(4-methyl-3,4,5,6-tetrahydro-2H- | ++ |
| [1,3′]bipyridinyl-6′-yl)-amine | |
| 6′-(6-Chloro-1H-benzoimidazol-2-yl)-5-methyl-[2,2′]bipyridinyl | ++ |
| [5-(2-Methoxy-phenyl)-pyridin-2-yl]-(6-thiazol-2-yl-pyridin-2-yl)-amine | ++ |
| (5-Cyclopropyl-[1,3,4]thiadiazol-2-yl)-(6-thiazol-2-yl-pyridin-2-yl)-amine | ++ |
| (5-tert-Butyl-pyridin-2-yl)-(6-thiazol-2-yl-pyridin-2-yl)-amine | ++ |
| 6-(6-Pyrazol-1-yl-pyridin-2-ylamino)-nicotinic acid methyl ester | ++ |
| (5-Isopropyl-pyridin-2-yl)-(3-methoxy-6-pyrazin-2-yl-pyridin-2-yl)-amine | ++ |
| [6-(5-Chloro-thiazol-2-yl)-pyridin-2-yl]-(3,4,5,6-tetrahydro-2H-[1,3′]bipyridinyl- | ++ |
| 6′-yl)-amine | |
| (3-Methoxy-6-thiazol-2-yl-pyridin-2-yl)-(5-phenyl-pyridin-2-yl)-amine | ++ |
| [6-(5-Methyl-thiazol-2-yl)-pyridin-2-yl]-(3,4,5,6-tetrahydro-2H-[1,3′]bipyridinyl- | ++ |
| 6′-yl)-amine | |
| [6-(5-Chloro-thiazol-2-yl)-3-methoxy-pyridin-2-yl]-(3,4,5,6-tetrahydro-2H- | ++ |
| [1,3′]bipyridinyl-6′-yl)-amine | |
| 4-Methyl-1-[6-(6-thiazol-2-yl-pyridin-2-ylamino)-pyridin-3-yl]-piperazin-2-one | ++ |
| 6′-[6-(5-Ethyl-thiazol-2-yl)-pyridin-2-ylamino]-3,4,5,6-tetrahydro- | ++ |
| [1,3′]bipyridinyl-2-one | |
| (5-Isopropyl-pyridin-2-yl)-(3-methoxy-6-thiazol-2-yl-pyridin-2-yl)-amine | ++ |
| 5-[(Benzylamino)methyl]-N-[6-(1,3-thiazol-2-yl)pyridin-2-yl]pyridin-2-amine | ++ |
| dihydrochloride | |
| (5-Cyclopropyl-[1,3,4]thiadiazol-2-yl)-(6-thiazol-2-yl-pyridin-2-yl)-amine | ++ |
| (3-Methoxy-6-thiazol-2-yl-pyridin-2-yl)-[5-(4-methyl-[1,4]diazepan-1-yl)- | ++ |
| pyridin-2-yl]-amine | |
| 1-Methyl-4-[6-(6-thiazol-2-yl-pyridin-2-ylamino)-pyridin-3-yl]-[1,4]diazepan-5- | ++ |
| one | |
| 1-[6-(6-Thiazol-2-yl-pyridin-2-ylamino)-pyridin-3-yl]-piperazin-2-one | ++ |
| N⁵-(1-Aza-bicyclo[2.2.2]oct-3-yl)-N²-(3-methoxy-6-thiazol-2-yl-pyridin-2-yl)- | ++ |
| pyridine-2,5-diamine | |
| N²-(3-Methoxy-6-thiazol-2-yl-pyridin-2-yl)-N⁵-methyl-N⁵-(1-methyl-pyrrolidin-3- | ++ |
| yl)-pyridine-2,5-diamine | |
| (3-Methoxy-6-thiazol-2-yl-pyridin-2-yl)-[5-(3-methyl-piperazin-1-yl)-pyridin-2- | ++ |
| yl]-amine | |
| [5-(4-Methyl-piperazin-1-yl)-pyridin-2-yl]-(6-pyrazol-1-yl-pyridin-2-yl)-amine | + |
| (6-Fluoro-pyridin-2-yl)-(6-thiazol-2-yl-pyridin-2-yl)-amine | + |

Key:
+ indicates 1.0 μM > IC50 > 0.5 μM;
++ indicates 0.5 μM > IC50 > 0.1 μM;
+++ indicates 0.1 μM > IC50.

### Example 25

25.1 Assay for Compound Activity in an Electroconvulsive Shock-Passive/Avoidance Model

The effects of compounds of the invention were studied on learning and memory formation for a passive avoidance task in mice following electroconvulsive shock training utilizing a modification of the protocol described by Inan, et al., Eur. J. Pharmacol., (2000), 407(1-2): 159-64.

25.2 Results

Compounds tested in this assay, along with their in vivo inhibitory activity, are provided in Table 4.

TABLE 4

| | In Vivo Inhibitory |
|---|---|
| | Activity |
| | (minimum |
| | effective dose, |
| Compound Name | (MED)) |
| (6-Thiazol-2-yl-pyridin-2-yl)-(5-thiophen-3-yl-pyridin-2-yl)-amine | ++ |
| (3-Methoxy-6-thiazol-2-yl-pyridin-2-yl)-[5-(4-methyl-piperazin-1-yl)- | ++ |
| pyridin-2-yl]-amine | |
| (5,6,7,8-Tetrahydro-isoquinolin-3-yl)-(6-thiazol-2-yl-pyridin-2-yl)-amine | ++ |
| (3-Methoxy-6-thiazol-2-yl-pyridin-2-yl)-(3,4,5,6-tetrahydro-2H- | ++ |
| [1,3′]bipyridinyl-6′-yl)-amine | |
| (3-Methoxy-6-thiazol-2-yl-pyridin-2-yl)-(5-morpholin-4-yl-pyridin-2-yl)- | ++ |
| amine | |
| (5-Pyrrolidin-1-ylmethyl-pyridin-2-yl)-(6-thiazol-2-yl-pyridin-2-yl)-amine | ++ |
| 1-{6-[6-(5-Chloro-thiazol-2-yl)-pyridin-2-ylamino]-pyridin-3-yl}-pyrrolidin- | ++ |
| 2-one | |
| 4-Methyl-1-[6-(6-thiazol-2-yl-pyridin-2-ylamino)-pyridin-3-yl]-piperazin-2- | ++ |
| one | |
| [6-(5-Chloro-thiazol-2-yl)-3-methoxy-pyridin-2-yl]-(5-pyrrolidin-1-yl- | ++ |
| pyridin-2-yl)-amine | |
| [5-(1,3-Dihydro-isoindol-2-ylmethyl)-pyridin-2-yl]-(6-thiazol-2-yl-pyridin-2- | ++ |
| yl)-amine | |
| 1-Methyl-4-[6-(6-thiazol-2-yl-pyridin-2-ylamino)-pyridin-3-yl]- | ++ |
| [1,4]diazepan-5-one | |
| (3-Methoxy-6-thiazol-2-yl-pyridin-2-yl)-(5-pyrrolidin-1-yl-pyridin-2-yl)- | ++ |
| amine | |
| (5-Phenyl-pyridin-2-yl)-(6-thiazol-2-yl-pyridin-2-yl)-amine | ++ |
| (5-Bromo-pyridin-2-yl)-[6-(4-methyl-pyrazol-1-yl)-pyridin-2-yl]-amine | ++ |
| (5-Chloro-pyridin-2-yl)-(6-pyrazin-2-yl-pyridin-2-yl)-amine | ++ |
| [5-(3-Fluoro-phenyl)-pyridin-2-yl]-[6-(4-methyl-pyrazol-1-yl)-pyridin-2-yl]- | ++ |
| amine | |
| 1-[6-(6-Thiazol-2-yl-pyridin-2-ylamino)-pyridin-3-yl]-piperazin-2-one | ++ |
| 1-[6-(3-Methoxy-6-thiazol-2-yl-pyridin-2-ylamino)-pyridin-3-yl]-pyrrolidin- | ++ |
| 2-one | |
| [6-(5-Chloro-thiazol-2-yl)-pyridin-2-yl]-(3,4,5,6-tetrahydro-2H- | ++ |
| [1,3′]bipyridinyl-6′-yl)-amine | |
| (5-Chloro-pyridin-2-yl)-(6-thiazol-2-yl-pyridin-2-yl)-amine | + |
| [5-(2-Methoxy-phenyl)-pyridin-2-yl]-(6-thiazol-2-yl-pyridin-2-yl)-amine | + |
| (5-Chloro-pyridin-2-yl)-(6-pyrazol-1-yl-pyridin-2-yl)-amine | + |
| (5-Methyl-pyridin-2-yl)-(6-thiazol-2-yl-pyridin-2-yl)-amine | + |

Key:
+ indicates 100.0 mg/kg ip > MED > 2.0 mg/kg ip;
++ indicates 2.0 mg/kg ip > MED.

### Example 26

26.1 Compound List

Table 5 below sets forth representative compounds of the invention with mass spec characterization data.

TABLE 5

| Compound # | Compound Name | M + 1 |
|---|---|---|
| 1. | (6′-Bromo-[2,2′]bipyridinyl-6-yl)-pyridin-2-yl-amine | 327 |
| 2. | N⁶,N^{6′}-Di-pyridin-2-yl-[2,2′]bipyridinyl-6,6′-diamine | 341 |
| 3. | N,N′-Di-pyridin-2-yl-pyridine-2,6-diamine | 264 |
| 4. | (6′-Bromo-[2,2′]bipyridinyl-6-yl)-(5-fluoro-pyridin-2-yl)-amine | 345 |
| 5. | (6′-Bromo-[2,2′]bipyridinyl-6-yl)-(5-chloro-pyridin-2-yl)-amine | 361 |
| 6. | (6′-Bromo-[2,2′]bipyridinyl-6-yl)-(4-methyl-pyridin-2-yl)-amine | 341 |
| 7. | [2,2′]Bipyridinyl-6-yl-(4-methyl-pyridin-2-yl)-amine | 263 |
| 8. | {[2,2′]Bipyridinyl-6-yl-pyridin-2-yl-amine}₂ Zn(II) Complex | 551 |
| 9. | 2-Amino-[1,2′;6′,2″]terpyridin-1-ylium; bromide | 264 |
| 10. | (6′-Bromo-[2,2′]bipyridinyl-6-yl)-methyl-pyridin-2-yl-amine | 341 |
| 11. | N,N′-Dimethyl-N,N′-di-pyridin-2-yl-pyridine-2,6-diamine | 292 |
| 12. | (6′-Bromo-[2,2′]bipyridinyl-6-yl)-(5-phenyl-pyridin-2-yl)-amine | 403 |
| 13. | [2,2′]Bipyridinyl-6-yl-methyl-pyridin-2-yl-amine | 263 |
| 14. | [2,2′]Bipyridinyl-6-yl-(5-phenyl-pyridin-2-yl)-amine | 325 |
| 15. | [2,2′]Bipyridinyl-6-yl-(5-iodo-pyridin-2-yl)-amine | 375 |
| 16. | (5′-Chloro-[2,2′]bipyridinyl-6-yl)-(5-chloro-pyridin-2-yl)-amine | 317 |
| 17. | (5-Chloro-pyridin-2-yl)-(5′-trifluoromethyl-[2,2′]bipyridinyl-6-yl)-amine | 351 |
| 18. | (5-Chloro-pyridin-2-yl)-(5′-morpholin-4-yl-[2,2′]bipyridinyl-6-yl)-amine | 368 |
| 19. | [2,2′]Bipyridinyl-6-yl-[5-(3-fluoro-phenyl)-pyridin-2-yl]-amine | 343 |
| 20. | [2,2′]Bipyridinyl-6-yl-[5-(2-fluoro-phenyl)-pyridin-2-yl]-amine | 343 |
| 21. | [6-(5-Methyl-[1,2,4]oxadiazol-3-yl)-pyridin-2-yl]-(5-phenyl-pyridin-2-yl)- | 330 |
| | amine | |
| 22. | (5-Chloro-pyridin-2-yl)-[6-(5-methyl-[1,2,4]oxadiazol-3-yl)-pyridin-2-yl]- | 288 |
| | amine | |
| 23. | (5-Chloro-pyridin-2-yl)-(6-pyrimidin-2-yl-pyridin-2-yl)-amine | 284 |
| 24. | (5-Chloro-pyridin-2-yl)-(6-thiazol-2-yl-pyridin-2-yl)-amine | 289 |
| 25. | [2,2′]Bipyridinyl-6-yl-pyrazin-2-yl-amine | 250 |
| 26. | [2,2′]Bipyridinyl-6-yl-(5-iodo-4-methyl-pyridin-2-yl)-amine | 389 |
| 27. | [2,2′]Bipyridinyl-6-yl-(5-iodo-3-methyl-pyridin-2-yl)-amine | 389 |
| 28. | (5-Chloro-pyridin-2-yl)-(6-pyrazol-1-yl-pyridin-2-yl)-amine | 272 |
| 29. | (5-Phenyl-pyridin-2-yl)-(6-pyrazol-1-yl-pyridin-2-yl)-amine | 314 |
| 30. | [2,2′]Bipyridinyl-6-yl-(5-iodo-pyridin-2-yl)-carbamic acid tert-butyl ester | 475 |
| 31. | [2,2′]Bipyridinyl-6-yl-(5-iodo-4-methyl-pyridin-2-yl)-carbamic acid tert- | 489 |
| | butyl ester | |
| 32. | [2,2′]Bipyridinyl-6-yl-[5-(4-fluoro-phenyl)-4-methyl-pyridin-2-yl]-carbamic | 457 |
| | acid tert-butyl ester | |
| 33. | (5-Phenyl-pyridin-2-yl)-(6-pyrimidin-2-yl-pyridin-2-yl)-amine | 326 |
| 34. | (5-Phenyl-pyridin-2-yl)-(6-pyrazin-2-yl-pyridin-2-yl)-amine | 326 |
| 35. | N-[2,2′]Bipyridinyl-6-yl-N-(5-iodo-pyridin-2-yl)-acetamide | 417 |
| 36. | 4-Methyl-6-(4-methyl-pyridin-2-ylamino)-[2,2′]bipyridinyl-5-carbonitrile | 302 |
| 37. | 4-Methyl-6-(pyridin-2-ylamino)-[2,2′]bipyridinyl-5-carbonitrile | 288 |
| 38. | 6-(5-Chloro-pyridin-2-ylamino)-4-methyl-[2,2′]bipyridinyl-5-carbonitrile | 322 |
| 39. | 6-(5-Fluoro-pyridin-2-ylamino)-4-methyl-[2,2′]bipyridinyl-5-carbonitrile | 306 |
| 40. | 6-(3,5-Dichloro-pyridin-2-ylamino)-4-methyl-[2,2′]bipyridinyl-5- | 356 |
| | carbonitrile | |
| 41. | (5-Fluoro-pyridin-2-yl)-[6-(4-methyl-pyrazol-1-yl)-pyridin-2-yl]-amine | 270 |
| 42. | [2,2′]Bipyridinyl-6-yl-[5-(4-dimethylamino-phenyl)-4-methyl-pyridin-2-yl]- | 382 |
| | amine | |
| 43. | (5-Chloro-pyridin-2-yl)-[6-(4-methyl-pyrazol-1-yl)-pyridin-2-yl]-amine | 286 |
| 44. | [5-(4-Fluoro-phenyl)-4-methyl-pyridin-2-yl]-(6-pyrazol-1-yl-pyridin-2-yl)- | 346 |
| | amine | |
| 45. | [4-Methyl-5-(4′-dimethyl amino)phenyl-pyridin-2-yl]-(6-pyrazol-1-yl- | 371 |
| | pyridin-2-yl)-amine | |
| 46. | 2,6-Bis-thiazol-2-yl-pyridine | 246 |
| 47. | (5-Bromo-3,4-dimethyl-pyridin-2-yl)-(6-thiazol-2-yl-pyridin-2-yl)-amine | 361 |
| 48. | (5-Bromo-pyrimidin-2-yl)-(6-thiazol-2-yl-pyridin-2-yl)-amine | 334 |
| 49. | (5-Fluoro-pyridin-2-yl)-(6-thiazol-2-yl-pyridin-2-yl)-amine | 273 |
| 50. | (5-Bromo-pyridin-2-yl)-(6-pyrazol-1-yl-pyridin-2-yl)-amine | 316 |
| 51. | (5-Bromo-pyridin-2-yl)-[6-(4-methyl-pyrazol-1-yl)-pyridin-2-yl]-amine | 330 |
| 52. | (4-Methyl-3,4,5,6-tetrahydro-2H-[1,3′]bipyridinyl-6′-yl)-(6-thiazol-2-yl- | 352 |
| | pyridin-2-yl)-amine | |
| 53. | (5-Fluoro-pyridin-2-yl)-(6-pyrazol-1-yl-pyridin-2-yl)-amine | 256 |
| 54. | (5-Morpholin-4-yl-pyridin-2-yl)-(6-thiazol-2-yl-pyridin-2-yl)-amine | 340 |
| 55. | (6-Thiazol-2-yl-pyridin-2-yl)-(5-thiophen-2-yl-pyridin-2-yl)-amine | 337 |
| 56. | [3,3′]Bipyridinyl-6-yl-(6-thiazol-2-yl-pyridin-2-yl)-amine | 332 |
| 57. | (5-Isopropenyl-pyridin-2-yl)-(6-thiazol-2-yl-pyridin-2-yl)-amine | 295 |
| 58. | (5-Isopropyl-pyridin-2-yl)-[6-(4-methyl-pyrazol-1-yl)-pyridin-2-yl]-amine | 294 |
| 59. | (5-Fluoro-pyridin-2-yl)-(6-pyrazin-2-yl-pyridin-2-yl)-amine | 268 |
| 60. | (5-Bromo-pyridin-2-yl)-(6-pyrazin-2-yl-pyridin-2-yl)-amine | 328 |
| 61. | [3,3′]Bipyridinyl-6-yl-(6-pyrazin-2-yl-pyridin-2-yl)-amine | 327 |
| 62. | (5-Isopropenyl-pyridin-2-yl)-(6-pyrazin-2-yl-pyridin-2-yl)-amine | 290 |
| 63. | (6-Pyrazin-2-yl-pyridin-2-yl)-(5-thiophen-2-yl-pyridin-2-yl)-amine | 332 |
| 64. | (5-Isopropyl-pyridin-2-yl)-(6-thiazol-2-yl-pyridin-2-yl)-amine | 297 |
| 65. | [6-(4-Methyl-pyrazol-1-yl)-pyridin-2-yl]-(5-phenyl-pyridin-2-yl)-amine | 328 |
| 66. | (5-Isopropenyl-pyridin-2-yl)-[6-(4-methyl-pyrazol-1-yl)-pyridin-2-yl]- | 292 |
| | amine | |
| 67. | (5-Methoxy-pyridin-2-yl)-(6-thiazol-2-yl-pyridin-2-yl)-amine | 285 |
| 68. | [5-(4-Methyl-piperazin-1-yl)-pyridin-2-yl]-(6-thiazol-2-yl-pyridin-2-yl)- | 353 |
| | amine | |
| 69. | [6-(4-Methyl-pyrazol-1-yl)-pyridin-2-yl]-(4-methyl-3,4,5,6-tetrahydro-2H- | 349 |
| | [1,3′]bipyridinyl-6′-yl)-amine | |
| 70. | [5-(4-Methyl-piperazin-1-yl)-pyridin-2-yl]-[6-(4-methyl-pyrazol-1-yl)- | 350 |
| | pyridin-2-yl]-amine | |
| 71. | [5-(4-Methyl-piperazin-1-yl)-pyridin-2-yl]-(6-pyrazol-1-yl-pyridin-2-yl)- | 336 |
| | amine | |
| 72. | [5-(2-Methoxy-phenyl)-pyridin-2-yl]-(6-thiazol-2-yl-pyridin-2-yl)-amine | 361 |
| 73. | (5-Pyrrolidin-1-yl-pyridin-2-yl)-(6-thiazol-2-yl-pyridin-2-yl)-amine | 324 |
| 74. | [6-(4-Methyl-pyrazol-1-yl)-pyridin-2-yl]-(5-pyrrolidin-1-yl-pyridin-2-yl)- | 321 |
| | amine | |
| 75. | (6-Thiazol-2-yl-pyridin-2-yl)-(5-thiophen-3-yl-pyridin-2-yl)-amine | 337 |
| 76. | (5-Furan-2-yl-pyridin-2-yl)-(6-thiazol-2-yl-pyridin-2-yl)-amine | 321 |
| 77. | (6-Fluoro-pyridin-2-yl)-(6-thiazol-2-yl-pyridin-2-yl)-amine | 273 |
| 78. | [5-(2-Methoxy-phenyl)-pyridin-2-yl]-[6-(4-methyl-pyrazol-1-yl)-pyridin-2- | 358 |
| | yl]-amine | |
| 79. | (6-Thiazol-2-yl-pyridin-2-yl)-[2-(6-thiazol-2-yl-pyridin-2-yl)-2H-pyrazol-3- | 404 |
| | yl]-amine | |
| 80. | (5-Bromo-pyridin-2-yl)-(6-thiazol-2-yl-pyridin-2-yl)-amine | 333 |
| 81. | 6-([2,2′]Bipyridinyl-6-ylamino)-N,N-diethyl-nicotinamide | 348 |
| 82. | N,N-Diethyl-6-(6-pyrazin-2-yl-pyridin-2-ylamino)-nicotinamide | 349 |
| 83. | (4-Methyl-thiazol-2-yl)-(6-thiazol-2-yl-pyridin-2-yl)-amine | 275 |
| 84. | 1-[6-(6-Pyrazin-2-yl-pyridin-2-ylamino)-pyridin-3-yl]-pyrrolidin-2-one | 333 |
| 85. | N,N-Diethyl-6-(6-thiazol-2-yl-pyridin-2-ylamino)-nicotinamide | 354 |
| 86. | (6-Pyrazin-2-yl-pyridin-2-yl)-(5-pyrrolidin-1-yl-pyridin-2-yl)-amine | 319 |
| 87. | (6-Pyrazin-2-yl-pyridin-2-yl)-(5-thiophen-3-yl-pyridin-2-yl)-amine | 332 |
| 88. | (5-Furan-2-yl-pyridin-2-yl)-(6-pyrazin-2-yl-pyridin-2-yl)-amine | 316 |
| 89. | [5-(3-Fluoro-phenyl)-pyridin-2-yl]-(6-pyrazin-2-yl-pyridin-2-yl)-amine | 344 |
| 90. | [5-(2-Methoxy-phenyl)-pyridin-2-yl]-(6-pyrazin-2-yl-pyridin-2-yl)-amine | 356 |
| 91. | [5-(5-Methyl-furan-2-yl)-pyridin-2-yl]-(6-pyrazin-2-yl-pyridin-2-yl)-amine | 330 |
| 92. | (4-Methyl-3,4,5,6-tetrahydro-2H-[1,3′]bipyridinyl-6′-yl)-(6-pyrazin-2-yl- | 347 |
| | pyridin-2-yl)-amine | |
| 93. | (5-Ethoxy-6-fluoro-pyridin-2-yl)-(6-thiazol-2-yl-pyridin-2-yl)-amine | 317 |
| 94. | Isoquinolin-3-yl-(6-thiazol-2-yl-pyridin-2-yl)-amine | 305 |
| 95. | (4-Phenyl-thiazol-2-yl)-(6-thiazol-2-yl-pyridin-2-yl)-amine | 337 |
| 96. | (5-tert-Butyl-isoxazol-3-yl)-(6-thiazol-2-yl-pyridin-2-yl)-amine | 301 |
| 97. | (5-Cyclopropyl-[1,3,4]thiadiazol-2-yl)-(6-thiazol-2-yl-pyridin-2-yl)-amine | 302 |
| 98. | (5,6,7,8-Tetrahydro-isoquinolin-3-yl)-(6-thiazol-2-yl-pyridin-2-yl)-amine | 309 |
| 99. | [1,10]Phenanthrolin-2-yl-pyridin-2-yl-amine | 273 |
| 100. | (6-Thiazol-2-yl-pyridin-2-yl)-(5-trifluoromethyl-[1,3,4]thiadiazol-2-yl)- | 330 |
| | amine | |
| 101. | (6-Thiazol-2-yl-pyridin-2-yl)-[1,2,4]triazol-4-yl-amine | 245 |
| 102. | (5-tert-Butyl-isoxazol-3-yl)-(6-pyrazin-2-yl-pyridin-2-yl)-amine | 296 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound according to Formula I: 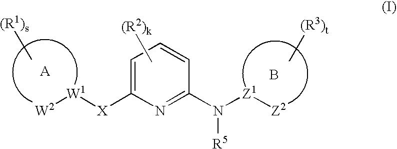
wherein
A and B are independently substituted or unsubstituted 5- or 6-membered heterocycloalkyl, or substituted or unsubstituted 5- or 6-membered heteroaryl,
wherein
W¹ and Z¹ are independently 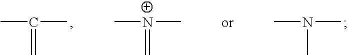
W² and Z² are independently —NH— or —N═;
X is a bond or —NR⁴—;
s and t are independently integers from 1 to 4;
k is an integer from 1 to 3;
R¹, R², and R³ are independently H, —NO₂, —CF₃, -L¹-OR⁶, -L²-NR⁷R⁸, -L³-CONR⁷R⁸, -L⁴-COOR⁶, -L⁵-COR⁶, -L⁶-SO₂R⁶, -L⁷-SO₂NR⁷R⁸, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl;
R⁴ and R⁵ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, -L³-CONR⁷R⁸, -L⁴-COOR⁶, -L⁵-COR⁶, -L⁶-SO₂R⁶, or -L⁷-SO₂NR⁷R⁸;
wherein
L¹, L², L³, L⁴, L⁵, L⁶, and L⁷ are independently a bond, or substituted or unsubstituted (C₁-C₆) alkylene;
R⁶ is H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl; and
R⁷ and R⁸ are independently H, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, —COR⁸¹, or —SO₂R⁸¹,
R⁸¹ is substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, or substituted or unsubstituted heteroaryl, wherein
R⁷ and R⁸ are optionally joined with the nitrogen to which they are attached to form a substituted or unsubstituted 5- to 7-membered heterocycloalkyl or substituted or unsubstituted heteroaryl;
wherein if s is greater than one, then each R¹ is optionally different;
wherein if k is greater than one, then each R² is optionally different;
wherein if t is greater than one, then each R³ is optionally different;
wherein two R¹ groups are optionally joined together with the atoms to which they are attached to form a substituted or unsubstituted 5- to 7-membered ring;
wherein two R² groups are optionally joined together with the atoms to which they are attached to form a substituted or unsubstituted 5- to 7-membered ring;
wherein two R³ groups are optionally joined together with the atoms to which they are attached to form a substituted or unsubstituted 5- to 7-membered ring;
wherein R¹ and R² are optionally joined together with the atoms to which they are attached to form a substituted or unsubstituted 5- to 7-membered ring;
wherein R² and R⁴ are optionally joined together with the atoms to which they are attached to form a substituted or unsubstituted 5- to 7-membered ring;
wherein R² and R⁵ are optionally joined together with the atoms to which they are attached to form a substituted or unsubstituted 5- to 7-membered ring;
wherein R² and R³ are optionally joined together with the atoms to which they are attached to form a substituted or unsubstituted 5- to 7-membered ring;
wherein R¹ and X are optionally joined together with the atoms to which they are attached to form a substituted or unsubstituted 5- to 7-membered ring;
wherein R² and X are optionally joined together with the atoms to which they are attached to form a substituted or unsubstituted 5- to 7-membered ring;
wherein R² and R⁵ are optionally joined together with the atoms to which they are attached to form a substituted or unsubstituted 5- to 7-membered ring; and
wherein R³ and R⁵ are optionally joined together with the atoms to which they are attached to form a substituted or unsubstituted 5- to 7-membered ring.

2. The compound of claim 1, wherein B is substituted or unsubstituted pyridinyl, substituted or unsubstituted 1,2,4-thiadiazolyl, substituted or unsubstituted pyrimidinyl, substituted or unsubstituted pyrazinyl, substituted or unsubstituted thiazolyl, substituted or unsubstituted isoxazolyl, or substituted or unsubstituted pyrazolyl.

3. The compound of claim 1, wherein B is substituted or unsubstituted pyridinyl.

4. The compound of claim 3, wherein Z¹ is 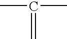
and Z² is —N═.

5. The compound of claim 1, wherein R⁵ is H.

6. The compound of claim 1, wherein X is a bond.

7. The compound of claim 6, wherein A is substituted or unsubstituted pyridinyl, substituted or unsubstituted pyrimidinyl, substituted or unsubstituted pyrazinyl, substituted or unsubstituted pyridazinyl, substituted or unsubstituted thiazolyl, substituted or unsubstituted isothiazolyl, substituted or unsubstituted benzimidazolyl, substituted or unsubstituted imidazolyl, substituted or unsubstituted pyrazolyl, or substituted or unsubstituted 1,2,4-oxadiazolyl.

8. The compound of claim 7, wherein A is substituted or unsubstituted pyridinyl, substituted or unsubstituted pyrazinyl, substituted or unsubstituted thiazolyl, or substituted or unsubstituted pyrazolyl.

9. The compound of claim 8, wherein A is unsubstituted pyridinyl, unsubstituted pyrazinyl, unsubstituted thiazolyl, unsubstituted pyrazolyl, or unsubstituted N-methylpyrazolyl.

10. The compound of claim 1, wherein R¹ is H, —OR⁶, —NR⁷R⁸, —NO₂, halogen, substituted or unsubstituted (C₁-C₅) alkyl, substituted or unsubstituted 2- to 5-membered heteroalkyl, substituted or unsubstituted 5- to 7-membered heterocycloalkyl, substituted or unsubstituted aryl, or substituted or unsubstituted heteroaryl.

11. The compound of claim 10, wherein R¹ is H, —NH₂, Br, F, Cl, —CF₃, methyl, —OCH₃, —NH—C(O)—CH₃, —NH—C(O)—CH₂CH₃ or unsubstituted morpholino.

12. The compound of claim 1, wherein k is 0.

13. The compound of claim 1, wherein R² is —CF₃, Cl, F, —OH, —NH₂, substituted or unsubstituted alkyl, or substituted or unsubstituted heteroalkyl.

14. The compound of claim 13, wherein R² is substituted or unsubstituted (C₁-C₆) alkyl.

15. The compound of claim 13, wherein R² is —CF₃, —OCH₃, —OCH(CH₃)₂,
—OCH₂CH₂OCH₃, —CH₂C(O)OCH₃, —OCH₂C(O)OCH₃, —C(O)N(CH₃)₂, —CN, —NHC(O)CH₃, or

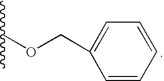

16. The compound of claim 1, wherein R³ is H, —OH, —NH₂, NO₂, —SO₂NH₂, halogen, substituted or unsubstituted alkyl, substituted or unsubstituted heteroalkyl, substituted or unsubstituted 5- to 7-membered cycloalkyl, substituted or unsubstituted 5- to 7-membered heterocycloalkyl, substituted or unsubstituted aryl, or substituted or unsubstituted heteroaryl.

17. The compound of claim 16, wherein R³ is substituted or unsubstituted pyrrolyl, substituted or unsubstituted thiazolyl, substituted or unsubstituted pyrrolidinonyl, substituted or unsubstituted pyridinyl, substituted or unsubstituted thiophenyl, substituted or unsubstituted furanyl, substituted or unsubstituted isoquinolinyl, or substituted or unsubstituted dihydroquinolinyl.

18. The compound of claim 16, wherein R³ is substituted or unsubstituted morpholino, substituted or unsubstituted thiomorpholino, substituted or unsubstituted pyrrolidinyl, substituted or unsubstituted pyrrolidinonyl, substituted or unsubstituted piperidinyl, substituted or unsubstituted piperazinyl, substituted or unsubstituted tetrahydrofuranyl, substituted or unsubstituted tetrahydropyranyl, substituted or unsubstituted tetrahydrothiophenyl, or substituted or unsubstituted tetrahydrothiopyranyl.

19. The compound of claim 1, wherein R³ is -L¹-OR⁶, -L²-NR⁷R⁸, -L³-CONR⁷R⁸, -L⁴-COOR⁶, or -L⁵-COR⁶
wherein
R⁶ is H, substituted or unsubstituted (C₁-C₆) alkyl, substituted or unsubstituted 2- to 6-membered heteroalkyl, substituted or unsubstituted 5- to 7-membered cycloalkyl, substituted or unsubstituted 5- to 7-membered heterocycloalkyl, substituted or unsubstituted heteroaryl, or substituted or unsubstituted aryl;
R⁷ and R⁸ are independently H, substituted or unsubstituted (C₁-C₆) alkyl, substituted or unsubstituted 2- to 6-membered heteroalkyl, or substituted or unsubstituted heteroaryl.

20. The compound of claim 19, wherein
R⁶ is H, unsubstituted (C₁-C₄) alkyl, —CH₂CH₂N(CH₃)₂, or unsubstituted benzyl;
R⁷ and R⁸ are independently H, methyl, ethyl, —C(O)CH₃ or unsubstituted pyridinyl;
wherein R⁷ and R⁸ are optionally joined with the nitrogen to which they are attached to form an unsubstituted pyrrolidinyl;
L¹ is a bond, methylene, ethylene, or propylene;
L² is a bond, methylene, or ethylene;
L³ is a bond;
L⁴ is a bond or ethylene;
L⁵ is a bond.

21. The compound of claim 20, wherein R³ is —OCH₃,
—OCH₂CH₃, 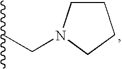
—C(═O)N(CH₃)₂, —C(═O)OCH₃, —(CH₂)₂C(═O)OCH₂CH₃, —CH₂OH, —(CH₂)₂OH, —(CH₂)₃OH, or —N(CH₃)(CH₂CH₂OCH₃).

22. The compound of claim 1, wherein R⁴ and R⁵ are independently H, substituted or unsubstituted alkyl, or substituted or unsubstituted heteroalkyl.

23. The compound of claim 22, wherein R⁴ and R⁵ are independently H, substituted or unsubstituted (C₁-C₆) alkyl, substituted or unsubstituted 2- to 6-membered heteroalkyl, or substituted or unsubstituted 5- to 7-membered heteroaryl.

24. The compound of claim 23, wherein R⁴ and R⁵ are independently H, methyl, —C(O)OC(CH₃)₃, —C(O)CH₃, or unsubstituted pyridinyl.

25. A metal complex, comprising a polyvalent metal ion and a polydentate component of a metal ion chelator, wherein said polydentate component is a compound according to claim 1.

26. The complex of claim 25, wherein said polyvalent metal ion is from iron, zinc, copper, cobalt, manganese, or nickel.

27. A method of decreasing ion flow through potassium ion channels in a cell, said method comprising contacting said cell with a potassium ion channel-modulating amount of a compound of one of claims 1-22, or 33-37, or a complex of one of claims 24 or 25.

28. The method according to claim 27, wherein said potassium ion channel comprises at least one SK subunit.

29. A method of treating a disease through modulation of a potassium ion channel, said method comprising administering to a subject in need of such treatment, an effective amount of a compound of one of claims 1-22, or 33-37, or a complex of one of claims 24 or 25.

30. The method according to claim 29, wherein said disorder or condition is selected from central or peripheral nervous system disorders, neuroprotective agents, gastroesophogeal reflux disorder, gastrointestinal hypomotility disorders, irritable bowel syndrome, secretory diarrhea, asthma, cystic fibrosis, chronic obstructive pulmonary disease, rhinorrhea, convulsions, vascular spasms, coronary artery spasms, renal disorders, polycystic kidney disease, bladder spasms, urinary incontinence, bladder outflow obstruction, ischemia, cerebral ischemia, ischemic heart disease, angina pectoris, coronary heart disease, Reynaud's disease, intermittent claudication, Sjorgren's syndrome, arrhythmia, hypertension, myotonic muscle dystrophia, xerostomi, diabetes type II, hyperinsulinemia, premature labor, baldness, cancer, and immune suppression.

31. The method according to claim 30, wherein said central or peripheral nervous system disorder comprises migraine, ataxia, Parkinson's disease, bipolar disorders, trigeminal neuralgia, spasticity, mood disorders, brain tumors, psychotic disorders, myokymia, seizures, epilepsy, hearing and vision loss, psychosis, anxiety, depression, dementia, memory and attention deficits, Alzheimer's disease, age-related memory loss, learning deficiencies, anxiety, traumatic brain injury, dysmenorrhea, narcolepsy and motor neuron diseases.

32. A pharmaceutical composition comprising a pharmaceutically acceptable carrier and a compound of one of claims 1-22, or 33-37, or a complex of one of claims 24 or 25.

33. The compound of claim 1, having the formula: 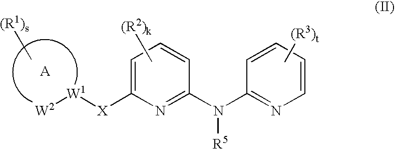
wherein
A is substituted or unsubstituted pyridinyl, substituted or unsubstituted pyrazinyl, substituted or unsubstituted thiazolyl, substituted or unsubstituted pyrimidinyl, substituted or unsubstituted imidazolyl, substituted or unsubstituted benzimidazolyl, or substituted or unsubstituted pyrazolyl,
R⁵ is H, substituted or unsubstituted alkyl, substituted or unsubstituted aryl, substituted or unsubstituted heteroaryl, —COR⁶, —COOR⁶, —CONR⁷R⁸, —SO₂R⁶, or —SO₂NR⁷R⁸; and
X is a bond.

34. The compound of claim 33, wherein A is substituted or unsubstituted thiazolyl.

35. The compound of claim 1, having the formula: 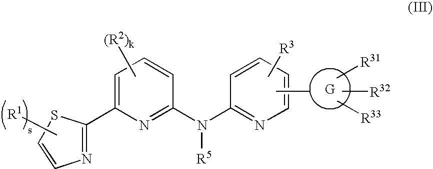
wherein
G is substituted or unsubstituted cyclopropyl, substituted or unsubstituted cyclobutyl, substituted or unsubstituted cyclopentyl, substituted or unsubstituted cyclohexyl, substituted or unsubstituted cycloheptyl, substituted or unsubstituted azetidinyl, substituted or unsubstituted pyrrolidinyl, substituted or unsubstituted piperidinyl, substituted or unsubstituted azepanyl, substituted or unsubstituted piperazinyl, substituted or unsubstituted morpholino, substituted or unsubstituted thiomorpholino, substituted or unsubstituted tetrahydropyridinyl, substituted or unsubstituted diazepanyl, substituted or unsubstituted furanyl, substituted or unsubstituted thienyl, substituted or unsubstituted pyrrolyl, substituted or unsubstituted thiazolyl, substituted or unsubstituted oxazolyl, substituted or unsubstituted pyrazolyl, substituted or unsubstituted oxadiazolyl, substituted or unsubstituted thiadiazolyl, substituted or unsubstituted triazolyl, substituted or unsubstituted tetrazolyl, substituted or unsubstituted phenyl, substituted or unsubstituted pyridinyl, substituted or unsubstituted pyrimidinyl, or substituted or unsubstituted pyrazinyl;
R³ is H, substituted or unsubstituted alkyl, —OR⁶, or halogen;
R⁵ is H, substituted or unsubstituted alkyl, substituted or unsubstituted aryl, or substituted or unsubstituted heteroaryl;
R³¹ and R³² are independently H, substituted or unsubstituted alkyl, —OR³¹¹, —NR³¹²R³¹³, —COR³¹¹, —COOR³¹¹, —CONR³¹²R³¹³, —SO₂R³¹¹, —SO₂NR³¹²R³¹³, oxo, NO₂, cyano, imino, or halogen;
R³³ is H, or substituted or unsubstituted alkyl;
R³¹² and R³¹³ are independently H, substituted or unsubstituted alkyl, substituted or unsubstituted aryl, —COR³¹⁴, or —SO₂R³¹⁴, wherein
R³¹⁴ is hydrogen, substituted or unsubstituted alkyl, or substituted or unsubstituted heteroalkyl; and
R³¹¹ is H, substituted or unsubstituted alkyl, or substituted or unsubstituted aryl.

36. The compound of claim 1, having the formula: 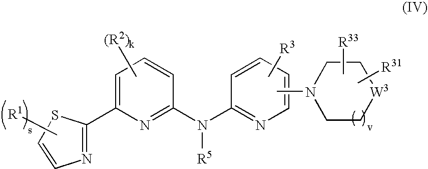
wherein
W³ is a bond, —O—, —S—, —N(R³²)—, or —C(R³⁴R³⁵)—;
v is an integer from 0 to 2;
R³ is H, substituted or unsubstituted alkyl, —OR⁶, or halogen;
R⁵ is H, substituted or unsubstituted alkyl, substituted or unsubstituted aryl, or substituted or unsubstituted heteroaryl;
R³¹, R³⁴, and R³⁵ are independently H, substituted or unsubstituted alkyl OR³¹¹, —NR³¹²R³¹³, —COR³¹¹, —COOR³¹¹, —CONR³¹²R³¹³, oxo, —NO₂, cyano, imino, or halogen;
R³² is H, alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, substituted or unsubstituted heteroaryl, —OR³¹¹, —COR³¹¹, —COOR³¹¹, —CONR³¹²R³¹³, —SO₂R³¹¹, —SO₂NR³¹²R³¹³, oxo, NO₂, cyano, imino, or halogen;
R³³ is H or substituted or unsubstituted alkyl;
R³¹² and R³¹³ are independently H, substituted or unsubstituted alkyl, substituted or unsubstituted aryl, —COR³¹⁴, or —SO₂R³¹⁴, wherein
R³¹⁴ is hydrogen, substituted or unsubstituted alkyl, or substituted or unsubstituted heteroalkyl; and
R³¹¹ is H, substituted or unsubstituted alkyl, or substituted or unsubstituted aryl.

37. The compound of claim 1, wherein said compound is:
(6-Thiazol-2-yl-pyridin-2-yl)-(5-thiophen-3-yl-pyridin-2-yl)-amine, (3-3 Methoxy-6-thiazol-2-yl-pyridin-2-yl)-[5-(4-methyl-piperazin-1-yl)-pyridin-2-yl]-amine, 4 (5,6,7,8-Tetrahydro-isoquinolin-3-yl)-(6-thiazol-2-yl-pyridin-2-yl)-amine, (3-Methoxy-6-thiazol-2-yl-pyridin-2-yl)-(3,4,5,6-tetrahydro-2H-[1,3′]bipyridinyl-6′-yl)-amine, (3-Methoxy-6-thiazol-2-yl-pyridin-2-yl)-(5-morpholin-4-yl-pyridin-2-yl)-amine, (5-Pyrrolidin-1-ylmethyl-pyridin-2-yl)-(6-thiazol-2-yl-pyridin-2-yl)-amine, 1-{6-[6-(5-Chloro-thiazol-2-yl)-pyridin-2-ylamino]-pyridin-3-yl}-pyrrolidin-2-one, 4-Methyl-1-[6-(6-thiazol-2-yl-pyridin-2-ylamino)-pyridin-3-yl]-piperazin-2-one, [6-(5-Chloro-thiazol-2-yl)-3-methoxy-pyridin-2-yl]-(5-pyrrolidin-1-yl-pyridin-2-yl)-amine, [5-(1,3-Dihydro-isoindol-2-ylmethyl)-pyridin-2-yl]-(6-thiazol-2-yl-pyridin-2-yl)-amine, 1-Methyl-4-[6-(6-thiazol-2-yl-pyridin-2-ylamino)-pyridin-3-yl]-[1,4]diazepan-5-one, (3-Methoxy-6-thiazol-2-yl-pyridin-2-yl)-(5-pyrrolidin-1-yl-pyridin-2-yl)-amine, (5-Phenyl-pyridin-2-yl)-(6-thiazol-2-yl-pyridin-2-yl)-amine, (5-Bromo-pyridin-2-yl)-[6-(4-methyl-pyrazol-1-yl)-pyridin-2-yl]-amine, (5-Chloro-pyridin-2-yl)-(6-pyrazin-2-yl-pyridin-2-yl)-amine, [5-(3-Fluoro-phenyl)-pyridin-2-yl]-[6-(4-methyl-pyrazol-1-yl)-pyridin-2-yl]-amine, 1-[6-(6-Thiazol-2-yl-pyridin-2-ylamino)-pyridin-3-yl]-piperazin-2-one, 1-[6-(3-Methoxy-6-thiazol-2-yl-pyridin-2-ylamino)-pyridin-3-yl]-pyrrolidin-2-one, or [6-(5-Chloro-thiazol-2-yl)-pyridin-2-yl]-(3,4,5,6-tetrahydro-2H-[1,3′]bipyridinyl-6′-yl)-amine.
